SEQUENCE LISTING (123 sequences)

SEQ ID NO: 1 - protein, 497 aa, Homo sapiens
  MOD_RES (495): R or H
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWXRQ

SEQ ID NO: 2 - DNA, 1551 bp, Homo sapiens
atggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagcactgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttaattgcccgtgacctaggtcctaccctcgccaacagtactcaccacaatgtccgcctactcatgctggatgaccaacgcttgctgctgccccactgggcaaaggtggtgctgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccatttcagcaagttcattcctgagggctcccagagagtggggctggttgccagtcagaagaacgacctggacgcagtggcattgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 3 - DNA, 6186 bp, Artificial sequence
  exon (1225)..(1572): Coding sequence for human FSH-alpha
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaaagcttggtaccgagctcggatccgccaccatggactactaccgcaagtacgccgccatcttcctggtgaccctgagcgtgttcctgcacgtgctgcacagcgcccccgacgtgcaggactgccccgagtgcaccctgcaggagaaccccttcttcagccagcccggcgcccccatcctgcagtgcatgggctgctgcttcagccgcgcctaccccacccccctgcgcagcaagaagaccatgctggtgcagaagaacgtgaccagcgagagcacctgctgcgtggccaagagctacaaccgcgtgaccgtgatgggcggcttcaaggtggagaaccacaccgcctgccactgcagcacctgctactaccacaagagctaatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - DNA, 5651 bp, Artificial sequence
  exon (1231)..(1617): Coding sequence for human FSH-beta
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaaagcttggtaccgagctcggatctatcgatgccaccatggagaccctgcagttcttcttcctgttctgctgctggaaggccatctgctgcaacagctgcgagctgaccaacatcaccatcgccatcgagaaggaggagtgccgcttctgcatcagcatcaacaccacctggtgcgccggctactgctacacccgcgacctggtgtacaaggaccccgcccgccccaagatccagaagacctgcaccttcaaggagctggtgtacgagacggtccgggtgcccggctgcgcccaccacgccgacagcctgtacacctaccccgtggccacccagtgccactgcggcaagtgcgacagcgacagcaccgactgcaccgtgcgcggcctgggccccagctactgcagcttcggcgagatgaaggagtaactcgagactagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - protein, 92 aa, Homo sapiens
APDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 6 - protein, 111 aa, Homo sapiens
NSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 7 - DNA, 6213 bp, Artificial sequence
  exon (1225)..(1599): Coding sequence for modified FSH-alpha
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaaagcttggtaccgagctcggatccgccaccatggactactaccgcaagtacgccgccatcttcctggtgaccctgagcgtgttcctgcacgtgctgcacagcgccaacatcaccgttaacatcaccgtggcccccgacgtgcaggactgccccgagtgcaccctgcaggagaaccccttcttcagccagcccggcgcccccatcctgcagtgcatgggctgctgcttcagccgcgcctaccccacccccctgcgcagcaagaagaccatgctggtgcagaagaacgtgaccagcgagagcacctgctgcgtggccaagagctacaaccgcgtgaccgtgatgggcggcttcaaggtggagaaccacaccgcctgccactgcagcacctgctactaccacaagagctaatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 8 - protein, 5 aa, Artificial Sequence
  Description of Artificial Sequence Synthetic peptide
ASNIX

SEQ ID NO: 9 - protein, 6 aa, Artificial Sequence
  Description of Artificial Sequence Synthetic peptide
SPINAX

SEQ ID NO: 10 - protein, 7 aa, Artificial Sequence
  Description of Artificial Sequence Synthetic peptide
ASPINAX

SEQ ID NO: 11 - protein, 11 aa, Artificial Sequence
  Description of Artificial Sequence Synthetic peptide
ANIXANIXANI

SEQ ID NO: 12 - protein, 14 aa, Artificial Sequence
  Description of Artificial Sequence Synthetic peptide
ANIXGSNIXGSNIX

SEQ ID NO: 13 - protein, 13 aa, Artificial Sequence
  Description of Artificial Sequence Synthetic peptide
ASNSXNNGXLNAX

SEQ ID NO: 14 - protein, 10 aa, Artificial Sequence
  Description of Artificial Sequence Synthetic peptide
ANHXNEXNAX

SEQ ID NO: 15 - protein, 7 aa, Artificial Sequence
  Description of Artificial Sequence Synthetic peptide
GSPINAX

SEQ ID NO: 16 - protein, 13 aa, Artificial Sequence
  Description of Artificial Sequence Synthetic peptide
ASPINAXSPINAX

SEQ ID NO: 17 - protein, 10 aa, Artificial Sequence
  Description of Artificial Sequence Synthetic peptide
ANNXNYXNWX

SEQ ID NO: 18 - protein, 13 aa, Artificial Sequence
  Description of Artificial Sequence Synthetic peptide
ATNIXLNYXANXT

SEQ ID NO: 19 - protein, 13 aa, Artificial Sequence
  Description of Artificial Sequence Synthetic peptide
AANSXGNIXINGX

SEQ ID NO: 20 - protein, 13 aa, Artificial Sequence
  Description of Artificial Sequence Synthetic peptide
AVNWXSNDXSNSX

SEQ ID NO: 21 - protein, 13 aa, Artificial Sequence
  Description of Artificial Sequence Synthetic peptide
AVNWXSNDXSNSX

SEQ ID NO: 22 - protein, 10 aa, Artificial Sequence
  Description of Artificial Sequence Synthetic peptide
ANNXNYXNSX

SEQ ID NO: 23 - protein, 10 aa, Artificial Sequence
  Description of Artificial Sequence Synthetic peptide
ANNTNYTNWT

SEQ ID NO: 24 - protein, 15 aa, Artificial Sequence
  Description of Artificial Sequence Linker
GGGGSGGGGSGGGGS

SEQ ID NO: 25 - DNA, 35 bp, Artificial Sequence
  Description of Artificial Sequence Primer
cgcagatctgatggctggcagcctcacaggattgc

SEQ ID NO: 26 - DNA, 37 bp, Artificial Sequence
  Description of Artificial Sequence Primer
ccggaattcccatcactggcgacgccacaggtaggtg

SEQ ID NO: 27 - DNA, 35 bp, Artificial Sequence
  Description of Artificial Sequence Primer
acgcgagctcgcccctgcatccctaaaagcttcgg

SEQ ID NO: 28 - DNA, 54 bp, Artificial Sequence
  Description of Artificial Sequence Primer
gcgttgacggcagtcagagttgacagaagggccagccagcaaaggatagtcatg

SEQ ID NO: 29 - DNA, 62 bp, Artificial Sequence
  Description of Artificial Sequence Primer
ctagcatgactatcctttgctggctggcccttctgtcaactctgactgccgtcaacgcagct

SEQ ID NO: 30 - DNA, 48 bp, Artificial Sequence
  Description of Artificial Sequence Primer
cctgctactgctcccagcagcagtgaaagagtccaaagtggcagcatg

SEQ ID NO: 31 - DNA, 56 bp, Artificial Sequence
  Description of Artificial Sequence Primer
ctagcatgctgccactttggactctttcactgctgctgggagcagtagcaggagct

SEQ ID NO: 32 - DNA, 21 bp, Artificial Sequence
  Description of Artificial Sequence Primer
cagctggccatgggtacccgg

SEQ ID NO: 33 - protein, 4 aa, Artificial Sequence
  Description of Artificial Sequence N-terminal peptide addition
ANIT

SEQ ID NO: 34 - protein, 7 aa, Artificial Sequence
  Description of Artificial Sequence N-terminal peptide addition
ASPINAT

SEQ ID NO: 35 - DNA, 48 bp, Artificial Sequence
  Description of Artificial Sequence Primer
tgggcatcaggtgccaacattacagcccgcccctgcatccctaaaagc

SEQ ID NO: 36 - DNA, 24 bp, Artificial Sequence
  Description of Artificial Sequence Primer
tttactgttttcgtaacagttttg

SEQ ID NO: 37 - DNA, 48 bp, Artificial Sequence
  Description of Artificial Sequence Primer
gcaggggcgggctgtaatgttggcacctgatgcccacgacactgcctg

SEQ ID NO: 38 - protein, 13 aa, Artificial Sequence
  Description of Artificial Sequence Synthetic peptide
AXNXTXNXTXNXT

SEQ ID NO: 39 - protein, 10 aa, Artificial Sequence
  Description of Artificial Sequence Synthetic peptide
ANXTNXTNXT

SEQ ID NO: 40 - DNA, 81 bp, Artificial Sequence
  modified_base (1)..(81): “n” represents a, t, c, g, other or unknown
gtgtcgtgggcatcaggtgccnnsaaydnsachdnsaaydnsachdnsaaydnsachgcccgcccctgcatccctaaaagc

SEQ ID NO: 41 - DNA, 27 bp, Artificial Sequence
  Description of Artificial Sequence Primer
ggcacctgatgcccacgacactgcctg

SEQ ID NO: 42 - DNA, 68 bp, Artificial Sequence
  Description of Artificial Sequence Primer
cgtgggcatcaggtgccaacnnnachaaynnnachaaynnnachgcccgcccctgcatccctaaaagc

SEQ ID NO: 43 - DNA, 30 bp, Artificial Sequence
  Description of Artificial Sequence Primer
gttggcacctgatgcccacgacactgcctg

SEQ ID NO: 44 - protein, 13 aa, Artificial Sequence
  Description of Artificial Sequence Synthetic peptide
AFNXTLNKTWNXT

SEQ ID NO: 45 - protein, 13 aa, Artificial Sequence
  Description of Artificial Sequence Synthetic peptide
TMNNTWNWTWNWT

SEQ ID NO: 46 - protein, 13 aa, Artificial Sequence
  Description of Artificial Sequence Synthetic peptide
ALNSTGNLTVDGT

SEQ ID NO: 47 - protein, 13 aa, Artificial Sequence
  Description of Artificial Sequence Synthetic peptide
ASNSTFNLTENLT

SEQ ID NO: 48 - protein, 12 aa, Artificial Sequence
  Description of Artificial Sequence Synthetic peptide
TRNVTINCTNST

SEQ ID NO: 49 - protein, 13 aa, Artificial Sequence
  Description of Artificial Sequence Synthetic peptide
ALNWTYNGTKNVT

SEQ ID NO: 50 - protein, 13 aa, Artificial Sequence
  Description of Artificial Sequence Synthetic peptide
AANWTVNFTGNFT

SEQ ID NO: 51 - protein, 12 aa, Artificial Sequence
  Description of Artificial Sequence Synthetic peptide
AXNXTVNSTNVT

SEQ ID NO: 52 - protein, 13 aa, Artificial Sequence
  Description of Artificial Sequence Synthetic peptide
ANNFTFNGTLNLT

SEQ ID NO: 53 - protein, 13 aa, Artificial Sequence
  Description of Artificial Sequence Synthetic peptide
AGNWTANVTVNVT

SEQ ID NO: 54 - protein, 13 aa, Artificial Sequence
  Description of Artificial Sequence Synthetic peptide
AGNSTSNVTGNWT

SEQ ID NO: 55 - protein, 13 aa, Artificial Sequence
  Description of Artificial Sequence Synthetic peptide
AVNSTMNIHAIPP

SEQ ID NO: 56 - protein, 13 aa, Artificial Sequence
  Description of Artificial Sequence Synthetic peptide
AGNGTVNGTINGT

SEQ ID NO: 57 - protein, 13 aa, Artificial Sequence
  Description of Artificial Sequence Synthetic peptide
AVNSTGNXTGNWT

SEQ ID NO: 58 - protein, 12 aa, Artificial Sequence
  Description of Artificial Sequence Synthetic peptide
AGNGTNGTSNLT

SEQ ID NO: 59 - protein, 13 aa, Artificial Sequence
  Description of Artificial Sequence Synthetic peptide
AMNSTKNSTLNIT

SEQ ID NO: 60 - protein, 10 aa, Artificial Sequence
  Description of Artificial Sequence Synthetic peptide
AFNYTSKNST

SEQ ID NO: 61 - protein, 13 aa, Artificial Sequence
  Description of Artificial Sequence Synthetic peptide
AVNATMNWTANGT

SEQ ID NO: 62 - protein, 13 aa, Artificial Sequence
  Description of Artificial Sequence Synthetic peptide
ASNSTNNGTLNAT

SEQ ID NO: 63 - protein, 13 aa, Artificial Sequence
  Description of Artificial Sequence Synthetic peptide
ARNKTKNFTINLT

SEQ ID NO: 64 - protein, 12 aa, Artificial Sequence
  Description of Artificial Sequence Synthetic peptide
APNITNDTVNMT

SEQ ID NO: 65 - protein, 13 aa, Artificial Sequence
  Description of Artificial Sequence Synthetic peptide
AQNKTFNFTMNCT

SEQ ID NO: 66 - protein, 13 aa, Artificial Sequence
  Description of Artificial Sequence Synthetic peptide
ALNVTWNCTLNLT

SEQ ID NO: 67 - protein, 10 aa, Artificial Sequence
  Description of Artificial Sequence Synthetic peptide
ALNTTWTNLT

SEQ ID NO: 68 - protein, 10 aa, Artificial Sequence
  Description of Artificial Sequence Synthetic peptide
ANTTNFTNET

SEQ ID NO: 69 - protein, 10 aa, Artificial Sequence
  Description of Artificial Sequence Synthetic peptide
ANWTNRTNCT

SEQ ID NO: 70 - protein, 10 aa, Artificial Sequence
  Description of Artificial Sequence Synthetic peptide
ANWTNFTNWT

SEQ ID NO: 71 - protein, 10 aa, Artificial Sequence
  Description of Artificial Sequence Synthetic peptide
PTGLIGTNFT

SEQ ID NO: 72 - protein, 10 aa, Artificial Sequence
  Description of Artificial Sequence Synthetic peptide
ANWTNKTNFT

SEQ ID NO: 73 - protein, 10 aa, Artificial Sequence
  Description of Artificial Sequence Synthetic peptide
ANNTNLTNAT

SEQ ID NO: 74 - protein, 10 aa, Artificial Sequence
  Description of Artificial Sequence Synthetic peptide
ANYTNWTNFT

SEQ ID NO: 75 - protein, 10 aa, Artificial Sequence
  Description of Artificial Sequence Synthetic peptide
ANTTNQTNDT

SEQ ID NO: 76 - protein, 10 aa, Artificial Sequence
  Description of Artificial Sequence Synthetic peptide
ANRTNWTNTT

SEQ ID NO: 77 - protein, 10 aa, Artificial Sequence
  Description of Artificial Sequence Synthetic peptide
PTATNHTNST

SEQ ID NO: 78 - protein, 10 aa, Artificial Sequence
  Description of Artificial Sequence Synthetic peptide
ANWTNQTNQT

SEQ ID NO: 79 - protein, 10 aa, Artificial Sequence
  Description of Artificial Sequence Synthetic peptide
ANWTNWTNAT

SEQ ID NO: 80 - protein, 10 aa, Artificial Sequence
  Description of Artificial Sequence Synthetic peptide
ANFTNKTNMT

SEQ ID NO: 81 - protein, 10 aa, Artificial Sequence
  Description of Artificial Sequence Synthetic peptide
ANHTNETNAT

SEQ ID NO: 82 - protein, 10 aa, Artificial Sequence
  Description of Artificial Sequence Synthetic peptide
ANXTNFTNET

SEQ ID NO: 83 - protein, 9 aa, Artificial Sequence
  Description of Artificial Sequence Synthetic peptide
ANLDKLHKH

SEQ ID NO: 84 - protein, 11 aa, Artificial Sequence
  Description of Artificial Sequence Synthetic peptide
ANCFTNQTNFT

SEQ ID NO: 85 - protein, 11 aa, Artificial Sequence
  Description of Artificial Sequence Synthetic peptide
ANWTNWTNEWT

SEQ ID NO: 86 - protein, 10 aa, Artificial Sequence
  Description of Artificial Sequence Synthetic peptide
ANCTNWTNCT

SEQ ID NO: 87 - protein, 10 aa, Artificial Sequence
  Description of Artificial Sequence Synthetic peptide
CHPYNWTNWT

SEQ ID NO: 88 - protein, 10 aa, Artificial Sequence
  Description of Artificial Sequence Synthetic peptide
ANETNYTNET

SEQ ID NO: 89 - protein, 7 aa, Artificial Sequence
  Description of Artificial Sequence Synthetic peptide
ANWTNWT

SEQ ID NO: 90 - protein, 10 aa, Artificial Sequence
  Description of Artificial Sequence Synthetic peptide
AKPYKSYKFY

SEQ ID NO: 91 - protein, 10 aa, Artificial Sequence
  Description of Artificial Sequence Synthetic peptide
ANITNKTNWT

SEQ ID NO: 92 - protein, 10 aa, Artificial Sequence
  Description of Artificial Sequence Synthetic peptide
ANWTNMTNIT

SEQ ID NO: 93 - protein, 10 aa, Artificial Sequence
  Description of Artificial Sequence Synthetic peptide
ANNTNRTNFT

SEQ ID NO: 94 - protein, 10 aa, Artificial Sequence
  Description of Artificial Sequence Synthetic peptide
ANWTNWTNWT

SEQ ID NO: 95 - protein, 11 aa, Artificial Sequence
  Description of Artificial Sequence Synthetic peptide
ANWRTNHTNKT

SEQ ID NO: 96 - protein, 10 aa, Artificial Sequence
  Description of Artificial Sequence Synthetic peptide
ANQTNITNWT

SEQ ID NO: 97 - protein, 11 aa, Artificial Sequence
  Description of Artificial Sequence Synthetic peptide
ANFTNVATNQT

SEQ ID NO: 98 - protein, 10 aa, Artificial Sequence
  Description of Artificial Sequence Synthetic peptide
ANTTXLTNKT

SEQ ID NO: 99 - protein, 10 aa, Artificial Sequence
  Description of Artificial Sequence Synthetic peptide
ANKTNXTNIT

SEQ ID NO: 100 - protein, 10 aa, Artificial Sequence
  Description of Artificial Sequence Synthetic peptide
ANWTNCTNIT

SEQ ID NO: 101 - protein, 10 aa, Artificial Sequence
  Description of Artificial Sequence Synthetic peptide
ANWTNXTNWT

SEQ ID NO: 102 - protein, 10 aa, Artificial Sequence
  Description of Artificial Sequence Synthetic peptide
CQLDRSTNET

SEQ ID NO: 103 - protein, 10 aa, Artificial Sequence
  Description of Artificial Sequence Synthetic peptide
ANNTNYTNWT

SEQ ID NO: 104 - protein, 10 aa, Artificial Sequence
  Description of Artificial Sequence Synthetic peptide
ANNTNYTNWT

SEQ ID NO: 105 - protein, 12 aa, Artificial Sequence
  Description of Artificial Sequence Synthetic peptide
AANDTNWTVNCT

SEQ ID NO: 106 - protein, 13 aa, Artificial Sequence
  Description of Artificial Sequence Synthetic peptide
ATNITLNYTANTT

SEQ ID NO: 107 - protein, 13 aa, Artificial Sequence
  Description of Artificial Sequence Synthetic peptide
AANSTGNITINGT

SEQ ID NO: 108 - protein, 13 aa, Artificial Sequence
  Description of Artificial Sequence Synthetic peptide
AVNWTSNDTSNST

SEQ ID NO: 109 - protein, 13 aa, Artificial Sequence
  Description of Artificial Sequence Synthetic peptide
ASPINATSPINAT

SEQ ID NO: 110 - protein, 4 aa, Artificial Sequence
  Description of Artificial Sequence Linker
GGGG

SEQ ID NO: 111 - protein, 4 aa, Artificial Sequence
  Description of Artificial Sequence Linker
GNAT

SEQ ID NO: 112 - protein, 8 aa, Artificial Sequence
  Description of Artificial Sequence Synthetic peptide
NSTQNATA

SEQ ID NO: 113 - protein, 14 aa, Artificial Sequence
  Description of Artificial Sequence Synthetic peptide
ANLTVRNLTRNVTV

SEQ ID NO: 114 - protein, 9 aa, Artificial Sequence
  Synthetic peptide
FNIXVNIXV

SEQ ID NO: 115 - protein, 9 aa, Artificial Sequence
  Synthetic peptide
YNIXVNIXV

SEQ ID NO: 116 - protein, 10 aa, Artificial Sequence
  Synthetic peptide
AFNIXVNIXV

SEQ ID NO: 117 - protein, 10 aa, Artificial Sequence
  Synthetic peptide
AYNIXVNIXV

SEQ ID NO: 118 - protein, 10 aa, Artificial Sequence
  Synthetic peptide
APNDXVNIXV

SEQ ID NO: 119 - protein, 9 aa, Artificial Sequence
  Synthetic peptide
ANIXVNIXV

SEQ ID NO: 120 - protein, 7 aa, Artificial Sequence
  Synthetic peptide
NDXVNFX

SEQ ID NO: 121 - protein, 8 aa, Artificial Sequence
  Synthetic peptide
NIXVNIXV

SEQ ID NO: 122 - protein, 12 aa, Artificial Sequence
  Synthetic peptide
APNDTVNFTQDC

SEQ ID NO: 123 - protein, 13 aa, Artificial Sequence
  Synthetic peptide
NSNITVNITVCEL

(19) United States
(12) Patent Application Publication
(10) Pub. No.: US 2006/0160177 A1
(43) Pub. Date: Jul. 20, 2006

(54) Peptide extended glycosylated polypeptides

(75) Inventors: Jens Sigurd Okkels, Vedbaek (DK); Anne Dam Jensen, Copenhagen (DK); Bart van den Hazel, Copenhagen (DK)

Correspondence Address: MAXYGEN, INC.;INTELLECTUAL PROPERTY DEPARTMENT, 515 GALVESTON DRIVE, RED WOOD CITY, CA, 94063, US

(73) Assignee: Maxygen ApS

(21) Appl. No.: 10/967,088

(22) Filed: Oct. 15, 2004

Related U.S. Application Data
(63) Continuation of application No. 09/896,896, filed on Jun. 29, 2001, now abandoned.
(60) Provisional application No. 60/225,558, filed on Aug. 16, 2000.
(60) Provisional application No. 60/217,497, filed on Jul. 11, 2000.

(30) Foreign Application Priority Data
Feb. 9, 2001 (WO) PCT/DK01/00090
Dec. 29, 2000 (WO) PCT/DK00/00743
Jul. 14, 2000 (DK) PA 2000 01092
Jun. 30, 2000 (DK) PA 2000 01027

(51) Int. Cl. C07H 21/04; C12P 21/06; C12P 19/34; C07K 9/00; C07K 14/47; C12N 5/06; C12N 1/18

(52) U.S. Cl. 435/69.1; 435/91.2; 435/320.1; 435/325; 435/348; 435/254.2; 530/322; 530/395; 536/23.5

(57) ABSTRACT

Glycosylated polypeptides comprising the primary structure NH₂-X-Pp-COOH, wherein X is a peptide addition comprising or contributing to a glycosylation site, and Pp is a polypeptide of interest or comprising the primary structure NH₂-Pₓ-X-P_{y}-COOH, wherein Pₓ is an N-terminal part of a polypeptide Pp of interest, P_{y} is a C-terminal part of said polypeptide Pp, and X is a peptide addition comprising or contributing to a glycosylation site are provided. The glycosylated polypeptides possess improved properties as compared to the polypeptide of interest.

### CROSS-REFERENCES TO RELATED APPLICATIONS

This application claims priority to and benefit of the following United States Provisional and International Patent Applications: Danish Patent Application PA 2000 01027, filed Jun. 30, 2000; U.S. Provisional Application 60/217,497, filed Jul. 11, 2000; PCT Application PCT/DK00/00743, filed Dec. 29, 2000; PCT Application PCT/DK01/00090, filed Feb. 9, 2001; Danish Patent Application PA 2000 01092, filed 14 Jul. 2000; and U.S. Provisional Application 60/225,558, filed Aug. 16, 2000, the specifications of which are each incorporated in their entirety for all purposes.

### FIELD OF THE INVENTION

The present invention relates to novel glycosylated polypeptides as well as means and methods for their preparation.

### BACKGROUND OF THE INVENTION

Polypeptides, including proteins, are used for a wide range of applications, including industrial uses and human or veterinary therapy.

One generally recognized drawback associated with many polypeptides is that they do not have a sufficiently high stability, are immunogenic or allergenic, have a reduced serum half-life, are susceptible to clearance, are susceptible to proteolytic degradation, and the like.

One method for improving properties of polypeptides has been to attach non-peptide moieties to the polypeptide to improve properties thereof. For instance, polymer molecules such as PEG has been used for reducing immunogenicity and/or increasing serum half-life of therapeutic polypeptides and for reducing allergenicity of industrial enzymes. Glycosylation has been suggested as another convenient route for improving properties of polypeptides such as stability, half-life, etc.

Machamer and Rose (1988) J. Biol. Chem. 263: 5948-5954 and 5955-5960, disclose modified glycoprotein G of vesicular stomatitis virus that is glycosylated at additional N-glycosylation sites introduced in the polypeptide backbone.

U.S. Pat. No. 5,218,092 discloses physiologically active polypeptides with at least one new or additional carbohydrate attached thereto. The additional carbohydrate molecule(s) is/are provided by adding one or more additional N-glycosylation sites to the polypeptide backbone, and expressing the polypeptide in a glycosylating host cell.

U.S. Pat. No. 5,041,376 discloses a method of identifying or shielding epitopes of a transportable protein, in which method an N-glycosylation site is introduced on the exposed surface of the protein backbone (using oligonucleotide-directed mutagenesis of the nucleotide sequence encoding the protein), the resulting protein is expressed, glycosylated and assayed for protein activity and for shielded epitopes.

WO 00/26354 discloses a method of reducing the allergenicity of proteins by including an additional glycosylation site in the protein backbone and glycosylating the resulting protein variant.

Guan et al. (1985) Cell 42: 489-496 disclose glycosylated fusion protein variants comprising a rat growth hormone backbone C-terminally extended with transmembrane and cytoplasmic domains of the vesicular stomatitis virus glycoprotein, which growth hormone backbone has been modified to incorporate two additional N-glycosylation sites.

WO 97/04079 discloses lipolytic enzymes modified to by an N- or C-terminal peptide extension capable of conferring improved performance, in particular wash performance to the enzyme.

Matsuura et al. (1999) Nature Biotechnology 17: 58-61 disclose the use of random elongation mutagenesis for improving thermostability of a non-glycosylated microbial catalase. The random elongation mutagenesis is conducted in the C-terminal end of the catalase.

U.S. Pat. No. 5,338,835, entitled CTP extended forms of FSH, describe the use of the C-terminal portion of the CG beta subunit or a variant thereof for extension of the C-terminal of CG, FSH and LH. Said C-terminal portion may comprise O-glycosylation sites. It is speculated that a similar approach may be used for other proteins.

U.S. Pat. No. 5,508,261 discloses alpha, beta-heterodimeric polypeptide having binding affinity to vertebrate luteinizing hormone (LH) receptors and vertebrate follicle stimulating hormone (FSH) receptors comprising a glycoprotein hormone alpha-subunit polypeptide and a specified non-naturally occurring beta-subunit polypeptide.

WO 95/05465 discloses EPO analogs which have one or more amino acids extending from the C-terminal end of EPO, the C-terminal extention having at least one additional carbohydrate site. The 28 amino acid C-terminal part of CG (having four O-glycosylation sites) is mentioned as an example.

WO 97/30161 discloses hybrid proteins comprising two coexpressed amino acid sequences forming a dimer, each comprising a) at least one amino acid sequence selected from a homomeric receptor, a chain of a heteromeric receptor, a ligand, and fragments thereof; and b) a subunit of a heterodimeric proteinaceous hormone or fragments thereof; in which a) and b) are joined directly or through a peptide linker, and, in each couple, the two subunits (b) are different and capable of aggregating to form a dimer complex.

In none of the above reference has it been disclosed or indicated that a polypeptide of interest can be modified to include additional glycosylation sites by N-terminally extending said polypeptide with a peptide sequence comprising one or more additional glycosylation sites. The present invention is based on this finding.

### SUMMARY OF THE INVENTION

Accordingly, in a first aspect the invention relates to a glycosylated polypeptide comprising the primary structure,
NH₂-X-Pp-COOH

wherein

X is a peptide addition comprising or contributing to a glycosylation site, and

Pp is a polypeptide of interest.

The introduction of additional glycosylation sites by means of a peptide addition is an elegant way of providing additional glycosylation sites in a polypeptide of interest. More specifically, the invention has the advantage that polypeptides with altered glycosylation pattern are more easily obtained, e.g., the variants can be designed without detailed knowledge or use of structural and/or functional properties of the polypeptide. Also, the utilization of glycosylation sites introduced by a peptide addition has been found to be improved relative to glycosylation sites introduced within a structural part of the polypeptide Pp. Also other properties of the peptide extended polypeptide, such as uptake in specific cells, may be improved relative to a polypeptide modified with glycosylation sites in a structural part (and not being subjected to peptide extension).

In a second aspect the invention relates to a glycosylated polypeptide comprising the primary structure NH₂-Pₓ-X-P_{y}-COOH, wherein

Pₓ is an N-terminal part of a polypeptide Pp of interest,

P_{y} is a C-terminal part of said polypeptide Pp, and

X is a peptide addition comprising or contributing to a glycosylation site.

In other aspects the invention relates to a nucleotide sequence encoding a polypeptide of the invention, an expression vector comprising said nucleotide sequence and methods of preparing a polypeptide of the invention.

In a further aspect the invention relates to a method of improving (a) selected property/ies of a polypeptide Pp of interest, which method comprises a) preparing a nucleotide sequence encoding a polypeptide comprising the primary structure
NH₂-X-Pp-COOH,

wherein

X is a peptide addition comprising or contributing to a glycosylation site, the peptide addition being capable of conferring the selected improved property/ies to the polypeptide Pp,

b) expressing the nucleotide sequence of a) in a suitable host cell under conditions ensuring attachment of an oligosaccharide moiety thereto, optionally

c) conjugating the expressed polypeptide of b) to a second non-peptide moiety, and,

d) recovering the polypeptide resulting from step c).

### BRIEF DESCRIPTION OF THE FIGURES

FIG. 1 is a dose response curve for uptake of glucocerebrosidase wildtype and modified according to the invention into J774E macrophages. The activity is measured by the GCB activity assay.

FIG. 2 illustrates the pharmakokinetics of a FSH polypeptide produced according to the invention.

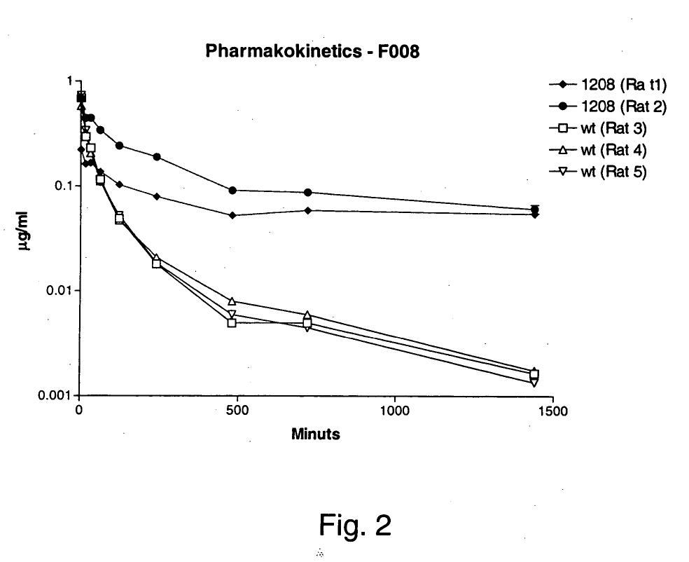

### DETAILED DISCUSSION

Definitions

In the context of the present application and invention the following definitions apply:

The term “conjugate” is used to refer to the covalent attachment of of one or more polypeptide(s) to one or more non-peptide moieties. The term covalent attachment means that the polypeptide and the non-peptide moiety are either directly covalently joined to one another, or else are indirectly covalently joined to one another through an intervening moiety or moieties, such as a bridge, spacer, or linkage moiety or moieties.

The term “non-peptide moiety” is intended to indicate a molecule, different from a peptide polymer composed of amino acid monomers and linked together by peptide bonds, which molecule is capable of conjugating to an attachment group of the polypeptide of the invention. Examples of such molecule include polymers e.g., polyalkylene oxide moieties lipophilic groups, e.g., fatty acids and ceramides. The term “polymer molecule” is defined as a molecule formed by covalent linkage of two or more monomers and may be used interchangeably with “polymeric group.” Except where the number of non-peptide moieties, such as polymeric groups, attached to the polypeptide is expressly indicated, every reference to “non-peptide moiety “referred to herein is intended as a reference to one or more non-peptide moieties attached to the polypeptide.

The term “oligosaccharide moiety” is intended to indicate a carbohydrate-containing molecule comprising one or more monosaccharide residues, capable of being attached to the polypeptide (to produce a glycosylated polypeptide) by way of in vivo or in vitro glycosylation. Except where the number of oligosaccharide moieties attached to the polypeptide is expressly indicated, every reference to “oligosaccharide moiety” referred to herein is intended as a reference to one or more such moieties attached to the polypeptide.

The term “in vivo glycosylation” is intended to mean-any attachment of an oligosaccharide moiety occurring in vivo, i.e., during posttranslational processing in a glycosylating cell used for expression of the polypeptide, e.g., by way of N-linked and O-linked glycosylation. Usually, the N-glycosylated oligosaccharide-moiety has a common basic core structure composed of five monosaccharide residues, namely two N-acetylglucosamine residues and three mannose residues. The exact oligosaccharide structure depends, to a large extent, on the glycosylating organism in question and on the specific polypeptide. Depending on the host cell in question the glycosylation is classified as a high mannose type, a complex type or a hybrid type. The term “in vitro glycosylation” is intended to refer to a synthetic glycosylation performed in vitro, normally involving covalently linking an oligosaccharide moiety to an attachment group of a polypeptide, optionally using a cross-linking agent. In vivo and in vitro glycosylation are discussed in detail further below.

An “N-glycosylation site” has the sequence N-X′-S/T/C-X”, wherein X′ is any amino acid residue except proline, X” any amino acid residue that may or may not be identical to X′ and preferably is different from proline, N asparagine and S/T/C either serine, threonine or cysteine, preferably serine or threonine, and most preferably threonine. The oligosaccharide moiety is attached to the N-residue of such site. An “O-glycosylation site” is the OH-group of a serine or threonine residue. An “in vitro glycosylation site” is, e.g., selected from the group consisting of the N-terminal amino acid residue of the polypeptide, the C-terminal residue of the polypeptide, lysine, cysteine, arginine, glutamine, aspartic acid, glutamic acid, serine, tyrosine, histidine, phenylalanine and tryptophan. Of particular interest is an in vitro glycosylation site that is an epsilon-amino group, in particular as part of a lysine residue.

The term “peptide addition” is intended to indicate one or more consecutive amino acid residues that are added to the amino acid sequence of the polypeptide Pp of interest. Normally, the peptide addition is linked to the amino acid sequence of the polypeptide Pp by a peptide linkage.

The term “attachment group” is intended to indicate a functional group of the polypeptide, in particular of an amino acid residue thereof or an oligosaccharide moiety attached to the polypeptide, capable of attaching a non-peptide moiety of interest. Useful attachment groups and their matching non-peptide moieties are apparent from the table below.

The term “comprising an attachment group” is intended to mean that the attachment group is present on an amino acid residue of the relevant peptide or polypeptide or on an oligosaccharide moiety attached to said peptide or polypeptide.

| | | | Conjugation | |
|---|---|---|---|---|
| Attachment | | Examples of non- | method/Activated | |
| group | Amino acid | peptide moiety | PEG | Reference |
| —NH₂ | N-terminal, | Polymer, e.g., PEG, | mPEG-SPA | Shearwater Inc. |
| | Lys | with amide or imine | Tresylated | Delgado et al, |
| | | group | mPEG | critical reviews |
| | | Lipophilic | | in Therapeutic |
| | | substituent | | Drug Carrier |
| | | | | Systems |
| | | | | 9(3, 4): 249-304 |
| | | | | (1992) |
| | | | | WO 97/31022 |
| —COOH | C-term, Asp, | Polymer, e.g., PEG, | mPEG-Hz | Shearwater Inc |
| | Glu | with ester or amide | | |
| | | group | | |
| —SH | Cys | Polymer, e.g., PEG, | PEG- | Shearwater Inc |
| | | with disulfide, | vinylsulphone | Delgado et al, |
| | | maleimide or vinyl | PEG-maleimide | critical reviews |
| | | sulfone group | | in Therapeutic |
| | | | | Drug Carrier |
| | | | | Systems |
| | | | | 9(3, 4): 249-304 |
| | | | | (1992) |
| —OH | Ser, Thr, | PEG with ester, | | |
| | OH—, Lys | ether, carbamate, | | |
| | | carbonate | | |
| —CONH₂ | | Polymer, e.g., PEG | | |
| Aldehyde | Oxidized | Polymer, e.g., PEG, | PEG-hydrazide | Andresz et al., |
| Ketone | oligosaccharide | | | 1978, |
| | | | | Makromol. |
| | | | | Chem. |
| | | | | 179: 301, WO |
| | | | | 92/16555, WO |
| | | | | 00/23114 |

The term “contributing to a glycosylation site” as used in connection with the peptide addition X is intended to cover the situation, where a glycosylation site is formed from more than one amino acid residue (as is the case with an N-glycosylation site), and where at least one such amino acid residue originates from the peptide X and at least one amino acid residue originates from the polypeptide Pp, whereby the glycosylation site can be considered to bridge X and Pp (or, where relevant, Pₓ or P_{y}).

The term “non-structural part” as used about a part of the polypeptide Pp of interest is intended to indicate a part of either the C- or N-terminal end of the folded polypeptide (e.g., protein) that is outside the first structural element, such as an α-helix or a β-sheet structure. The non-structural part can easily be identified in a three-dimensional structure or model of the polypeptide. If no structure or model is available, a non-structural part typically comprises or consists of the first or last 1-20 (1, 2, 3, 4, 5, 6, 7, 8, 9, 10, 11, 12, 13, 14, 15, 16, 17, 18, 19 or 20) amino acid residues, such as 1-10 amino acid residues of the amino acid sequence constituting the mature form of the polypeptide of interest.

Amino acid names and atom names (e.g., CA, CB, NZ, N, O, C, etc) are used as defined by the Protein DataBank (PDB) (www.pdb.org) which are based on the IUPAC nomenclature (IUPAC Nomenclature and Symbolism for Amino Acids and Peptides (residue names, atom names e.t.c.), Eur. J. Biochem., 138, 9-37 (1984) together with their corrections in Eur. J. Biochem., 152, 1 (1985). The term “amino acid residue” is intended to indicate an amino acid residue contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The terminology used for identifying amino acid positions/mutations is illustrated as follows: A15 (indicates an alanine residue in position 15 of the polypeptide), A15T (indicates replacement of the alanine residue in position 15 with a threonine residue), A15[T/S] (indicates replacement of the alanine residue in position 15 with a threonine residue or a serine residue). Multiple substitutions are indicated with a “+,” e.g., A15T+F57S means an amino acid sequence which comprises a substitution of the alanine residue in position 15 for a threonine residue and a substitution of the phenylalanine residue in position 57 for a serine residue.

The term “nucleotide sequence” is intended to indicate a consecutive stretch of two or more nucleotides. The nucleotide sequence can be of genomic, cDNA, RNA, semisynthetic, synthetic origin, or any combinations thereof.

“Cell,” “host cell,” “cell line” and “cell culture” are used interchangeably herein and all such terms should be understood to include progeny resulting from growth or culturing of a cell. “Transformation” and “transfection” are used interchangeably to refer to the process of introducing DNA into a cell.

“Operably linked” refers to the covalent joining of two or more nucleotide sequences in such a manner that the normal function of the sequences can be performed. For example, the nucleotide sequence encoding a presequence or secretory leader is operably linked to a nucleotide sequence for a polypeptide if it is expressed as a preprotein that participates in the secretion of the polypeptide: a promoter or enhancer is operably linked to a coding sequence if it affects the transcription of the sequence.

“Introduction” or “removal” of a glycosylation site or an attachment group for a non-peptide moiety is normally achieved by introducing or removing an amino acid residue comprising or contributing to such site or group to/from the relevant amino acid sequence, conveniently by suitable modification of the encoding nucleotide sequence. For instance, when an N-glycosylation site is to be introduced/removed this can be done by introducing/removing a codon for the amino acid residue(s) required for a functional N-glycosylation site. When an attachment group for a PEG molecule is to be introduced/removed, it will be understood that this be done by introducing/removing a codon for an amino acid residue, e.g., a lysine residue, comprising such group to/from the encoding nucleotide sequence. The term “introduce” is primarily intended to include substitution of an existing amino acid residue, but can also mean insertion of additional amino acid residue. The term “remove” is primarily intended to include substitution of the amino acid residue to be removed for another amino acid residue, but can also mean deletion (without substitution) of the amino acid residue to be removed.

The term “epitope” is used in its conventional meaning to indicate one or more amino acid residue(s) displaying specific 3D and/or charge characteristics at the surface of the polypeptide, which is/are capable of giving rise to an immune response in a mammal and/or specifically binding to an antibody raised against said epitope or which is/are capable of giving rise to an allergic response.

The term “unshielded epitope” is intended to indicate that the epitope is not shielded and therefore has the above properties. The term “shielded epitope” is intended to indicate that the non-peptide moiety shields, and thus inactivates the epitope, whereby it is no longer capable of giving rise to any substantial immune response in a mammal, e.g., due to inappropriate processing and/or presentation in the antigen presenting cells, and/or of reacting with an antibody raised against the unshielded epitope. The shielding should thus be effective in both the naïve mammal and mammals that already produce antibodies reacting with the unshielded epitope.

The degree of shielding of epitopes can be determined as reduced immunogenicity and/or reduced antibody reactivity and/or reduced reactivity with monoclonal antibodies raised against the epitope(s) in question using methods known in the art. The degree of shielding of allergenic epitopes can be determined, e.g., as described in WO 00/26354.

The term “reduced” as used about an immunogenic or allergic response is intended to indicate that a given molecule gives rise to a measurably lower immune or allergic response than a reference molecule, when determined under comparable conditions. Preferably, the relevant response is reduced by at least 25%, such as at least 50%, such as preferably by at least 75%, such as by at least 90% or even at least 100%.

The term “serum half-life” is used in its normal meaning, i.e., the time in which half of the relevant molecules circulate in the plasma or bloodstream prior to being cleared. Alternatively used terms include “plasma half-life,” “circulating half-life,” “serum clearance,” “plasma clearance” and “clearance half-life.” The term “functional in vivo half-life” is the time in which 50% of a given function (such as biological activity) of the relevant molecule is retained, when tested in vivo (such as the time at which 50% of the biological activity of the molecule is still present in the body/target organ, or the time at which the activity of the polypeptide is 50% of the initial value). The molecule is normally cleared by the action of one or more of the reticuloendothelial systems (RES), kidney (e.g., by glomerular filtration), spleen or liver, or receptor-mediated elimination, or degraded by specific or unspecific proteolysis. Normally, clearance depends on size or hydrodynamic volume (relative to the cut-off for glomerular filtration), shape/rigidity, charge, attached carbohydrate chains, and the presence of cellular receptors for the molecule. The term “increased” as used about serum half-life or functional in vivo half-life is used to indicate that the relevant half-life of the relevant molecule is statistically significantly increased relative to that of the reference molecule as determined under comparable conditions. For instance, the relevant half-life is increased by at least 25%, such as by at least 50%, by at least 100% or by at least 1000%.

The term “function” is intended to indicate one or more specific functions of the polypeptide of interest and is to be understood qualitatively (i.e., having a similar function as the polypeptide of interest) and not necessarily quantitatively (i.e., the magnitude of the function is not necessarily similar). Typically, a given polypeptide has many different functions, examples of which are given further below in the section entitled “Screening for or measurement of function.” For therapeutically useful polypeptides an important “function” is biological activity, e.g., in vitro or in vivo bioactivity. For enzymes, an important function is biological activity such as catalytic activity.

The interchangeably used terms “measurable function” and “functional” are intended to indicate that the relevant function (preferably reflecting the intended use) of a polypeptide of the invention is above detection limit when measured by standard methods known in the art, e.g., as an in vitro bioactivity and/or in vivo bioactivity. For instance, if the polypeptide is a hormone and the function of interest is the hormone's affinity towards a specific receptor a measurable function is defined to be a detectable affinity between the hormone modified in accordance with the invention and the receptor as determined by the normal methods used for measuring such affinity. If the polypeptide is an enzyme and a function of interest is the catalytic activity a measurable function is the enzyme's ability to catalyze a reaction involving the normal substrates for the enzyme as measured by the normal methods for determining the enzyme activity in question. Typically, if not otherwise stated herein, a measurable function is at least 2%, such as at least 5% of that of the unmodified polypeptide Pp, as determined under comparable conditions, e.g., in the range of 2-1000%, such as 2-500% or 2-100%, such as 5-100% of that of the unmodified polypeptide.

The term “functional site” is intended to indicate one or more amino acid residues which is/are essential for or otherwise involved in the function or performance of the polypeptide, i.e., the amino acid residue(s) that mediate(s) a desired biological activity of the polypeptide Pp. Such amino acid residues are “located at” the functional site. For instance, the functional site can be a binding site (e.g., a receptor-binding site of a hormone or growth factor or a ligand-binding site of a receptor), a catalytic site (e.g., of an enzyme), an antigen-binding site (e.g., of an antibody), a regulatory site (e.g., of a polypeptide subject to regulation), or an interaction site (e.g., for a regulatory protein or an inhibitor). The functional site can be determined by methods known in the art and is conveniently identified by analysing a three-dimensional or model structure of the polypeptide complexed to a relevant ligand.

The term “polypeptide” is intended to indicate any structural form (e.g., the primary, secondary or tertiary form (i.e., protein form)) of an amino acid sequence comprising more than 5 amino acid residues, which may or may not be post-translationally modified (e.g., acetylated, carboxylated, phosphorylated, lipidated, or acylated). The interchangeably used terms “native” and “wild-type” are used about a polypeptide which has an amino acid sequence that is identical to one found in nature. The native polypeptide is typically isolated from a naturally occurring source, in particular a mammalian or microbial source, such as a human source, or is produced recombinantly by use of a nucleotide sequence encoding the naturally occurring amino acid sequence. The term “native” is intended to encompass allelic variants of the polypeptide in question. A “variant” is a polypeptide, which has an amino acid sequence that differs from that of a native polypeptide in one or more amino acid residues. The variant is typically prepared by modification of a nucleotide sequence encoding the native polypeptide (e.g., to result in substitution, deletion or truncation of one or more amino acid residues of the polypeptide or by introduction (by addition or insertion) of one or more amino acid residues into the polypeptide) so as to modify the amino acid sequence constituting said native polypeptide. A “fragment” is a part of a parent native or variant polypeptide, typically differing from such parent in one or more removed C-terminal or N-terminal amino acid residues or removal of both types of such residues. Normally, the variant or fragment has retained at least one of the functions of the corresponding parent polypeptide (e.g., a biological function such as enzyme activity or receptor binding capability). Normally, the polypeptide Pp is a full length protein or a variant or fragment thereof.

The term “antibody” includes single monoclonal antibodies (including agonist and antagonist antibodies) and antibody compositions with polyepitopic specificity (also termed polyclonal antibodies).

The term “monoclonal antibody” is used in its conventional meaning to indicate a population of substantially homogeneous antibodies. The individual antibodies comprised in the population have identical binding affinities and vary structurally only to a limited extent. Monoclonal antibodies are highly specific, being directed against a single epitope. Furthermore, in contrast to conventional (polyclonal) antibody preparations that typically include different antibodies directed against different epitopes, each monoclonal antibody is directed against a single epitope on the antigen. The antibody to be modified is preferably a human or humanized monoclonal antibody.

“Antibody fragment” is defined as a portion of an intact antibody comprising the antigen binding site or the entire or part of the variable region of the intact antibody, wherein the portion is free of the constant heavy chain domains (i.e., CH2, CH3, and CH4, depending on antibody isotype) of the Fc regions of the intact antibody. Examples of antibody fragments include Fab, Fab′, Fab′-SH, F(ab′)2, and Fv fragments; diabodies; any antibody fragment that is a polypeptide having a primary structure consisting of one uninterrupted sequence of contiguous amino acid residues (which may also be termed a single chain antibody fragment or a single chain polypeptide).

Polypeptide of the Invention

In its first aspect the invention relates to a glycosylated polypeptide comprising the primary structure:
NH₂-X-Pp-COOH,
wherein X is a peptide addition comprising or contributing to a glycosylation site, and Pp is a polypeptide of interest.

In one embodiment, the polypeptide consists essentially of or consists of a polypeptide with the primary structure NH₂-X-Pp-COOH.

The peptide addition according to this aspect is preferably one, which has less than 90% identity to a native full length protein. The identity is determined on the basis of an alignment of the peptide addition to the entire amino acid sequence of the full length native protein, the alignment being made to ensure the highest possible degree of identity between amino acid residues. For instance, the program CLUSTALW version 1.74 using default parameters (Thompson et al. (1994) CLUSTAL W: improving the sensitivity of progressive multiple sequence alignment through sequence weighting, position-specific gap penalties and weight matrix choice Nucleic Acids Research 22:4673-4680) can be used.

Usually, the peptide addition is fused to the N-terminal end of the polypeptide Pp as reflected in the above shown structure so as to provide an N-terminal elongation of the polypeptide Pp. However, it is also possible to insert the peptide addition within the amino acid sequence of the polypeptide Pp. This is reflected in the polypeptide according to the second aspect of the invention, wherein the polypeptide comprises the primary structure NH₂-Pₓ-X-P_{y}-COOH, wherein

Pₓ is an N-terminal part of a polypeptide Pp of interest,

P_{y} is a C-terminal part of said polypeptide Pp, and

X is a peptide addition comprising or contributing to a glycosylation site.

In one embodiment, the polypeptide consists essentially of or consists of a polypeptide with the primary structure NH₂-Pₓ-X-P_{y}-COOH.

In order to minimize structural changes effected by the insertion of the peptide addition within the sequence of the polypeptide Pp, it is desirable that it be inserted in a non-structural part thereof. For instance, Pₓ is a non-structural N-terminal part of a mature polypeptide Pp, and P_{y} is a structural C-terminal part of said mature polypeptide, or Pₓ is a structural N-terminal part of a mature polypeptide Pp, and P_{y} is a non-structural C-terminal part of said mature polypeptide. Preferably, when the glycosylation site to be introduced is an N-glycosylation site, Pₓ is a non-structural N-terminal part since, in general, the best N-glycosylation is obtained in the N-terminal part of a polypeptide.

When the peptide addition comprises only few amino acid residues, e.g., 1-5 such as 1-3 amino acid residues, and in particular 1 amino acid residue, the peptide addition can be inserted into a loop structure of the polypeptide Pp and thereby elongate said loop. When the peptide addition is constituted by one amino acid residue it will be understood that this is selected so as to ensure that a functional glycosylation site is introduced.

Polypeptides of the invention are glycosylated polypeptides. Normally, the peptide addition part of the polypeptide of the invention has attached at least one oligosaccharide moiety. The polypeptide Pp part of the polypeptide may or may not have attached at least one oligosaccharide moiety. Glycosylation can be achieved as described in the section entitled “Glycosylation”

Preferably, the polypeptide of the invention has properties such as size, charge, molecular weight and/or hydrodynamic volume that are sufficient to reduce or escape clearance by any of the clearance mechanisms disclosed herein, in particular renal clerance. Such properties are, e.g., determinable by the nature and number of oligosaccharide and second non-peptide moieties attached thereto. In one embodiment, the polypeptide of the invention has a molecular weight of at least 67 kDa, in particular at least 70 kDa as measured by SDS-PAGE according to Laemmli, U.K., Nature Vol 227 (1970), p680-85. This is of particular relevance when the polypeptide of interest is a therapeutically useful protein, the functional in vivo half-life of which is to be prolonged. A molecular weight of at least 67 kDa is obtainable by introduction of a sufficient number of glycosylation sites to obtain a glycosylated polypeptide with such MW, or by conjugating the glycosylated polypeptide to a sufficient number and type of a second non-peptide moiety to obtain such MW. For instance, for a glycosylated polypeptide of interest having a molecular weight of at least 25 kDa linked to a peptide addition of 2 kDa, the combined extended polypeptide having at least two PEG-attachment groups, conjugation to two or more PEG molecules each having a molecular weight of 20 kDa results in a total molecular weight of at least 67 kDa.

Preferably, the polypeptide of the invention has at least one of the following properties relative to the polypeptide Pp, the properties being measured under comparable conditions: in vitro bioactivity which is at least 25%, such as at least 30% or at least 45% of that of the polypeptide Pp as measured under comparable conditions, increased affinity for a mannose receptor, a mannose-6-phosphate receptor or other carbohydrate receptors, increased serum half-life, increased functional in vivo half-life, reduced renal clearance, reduced immunogenicity, increased resistance to proteolytic cleavage, improved targeting to lysosomes, macrophages and/or other subpopulations of human cells, improved stability in production, improved shelf life, improved formulation, e.g., liquid formulation, improved purification, improved solubility, and/or improved expression.

Improved properties are determined by conventional methods known in the art for determining such properties. The improvement is of a magnitude that is within detection limits.

Improved affinity for or uptake by the mannose receptor is expected to result in increased uptake in phagocytic cells, preferably monocytes, macrophages (e.g., Kupffer cells, glia/microglia, alveolar phagocytes, reticulum cells, or other peripheral macrophages) or macrophage like cells (for instance osteoclasts, dendritic cells, or astrocytes) in increased uptake of the polypeptide in phagocytic cells (e.g., macrophages). This is of particular relevance when the polypeptide of interest is one for which such uptake is required for the polypeptide to exert its biological activity. Such polypeptide is e.g., an antigen intended for use for vaccine purposes or a lysosomal enzyme.

Polypeptide of Interest

The present invention can be applied broadly. Thus, the polypeptide of interest can have any function and be of any origin. Accordingly, the polypeptide can be a protein, in particular a mature protein or a precursor form thereof or a functional fragment thereof that essentially has retained a biological activity of the mature protein. Furthermore, the polypeptide can be an oligopeptide that contains in the range of 30 to 4500 amino acids, preferably in the range of 40 to 3000 amino acids.

The polypeptide can be a native polypeptide or a variant thereof. For instance, the polypeptide is a variant that comprises at least one introduced and/or at least one removed glycosylation site as compared to the corresponding native polypeptide. The variant has retained at least one function of the corresponding native polypeptide, in particular a biological activity thereof.

The polypeptide can be a therapeutic polypeptide useful in human or veterinary therapy, i.e., a polypeptide that is physiologically active when introduced into the circulatory system of or otherwise administered to a human or an animal; a diagnostic polypeptide useful in diagnosis; or an industrial polypeptide useful for industrial purposes, such as in the manufacture of goods wherein the polypeptide constitutes a functional ingredient or wherein the polypeptide is used for processing or other modification of raw ingredients during the manufacturing process.

The polypeptide can be of mammalian origin, e.g., of human, porcine, ovine, urcine, murine, rabbit, donkey, or bat origin, of microbial origin, e.g., of fungal, yeast or bacterial origin, or can be derived from other sources such as venom, leech, frog or mosquito origin. Preferably, the industrial polypeptide of interest is of microbial origin and the therapeutic polypeptide of human origin.

Specific examples of groups of polypeptides to be modified according to the invention include: an antibody or antibody fragment, an immunoglobulin or immunoglobulin fragment, a plasma protein, an erythrocyte or thrombocyte protein, a cytokine, a growth factor, a profibrinolytic protein, a binding protein, a protease inhibitor, an antigen, an enzyme, a ligand, a receptor, or a hormone. Of particular interest is a polypeptide that mediates its biological effect by binding to a cellular receptor, when administered to a patient. The antibody can be a polyclonal or monoclonal antibody, and can be of any origin including human, rabbit and murine origin. Preferably, the antibody is a human or humanized monoclonal antibody. Immunoglobulins of interest include IgG, IgE, IgM, IgA, and IgD and fragments thereof, e.g., Fab fragments. Specific antibodies and fragments thereof are those reactive with any of the proteins mentioned immediately below.

The non-antibody polypeptide of interest can be i) a plasma protein, e.g., a factor from the coagulation system, such as Factor VII, Factor VIII, Factor IX, Factor X, Factor XIII, thrombin, protein C, antithrombin III or heparin co-factor II, Tissue factor inhibitor (e.g., 1 or 2), endothelial cell surface protein C receptor, a factor from the fibrinolytic system such as pro-urokinase, urokinase, tissue plasminogen activator, plasminogen activator inhibitor 1 (PAI-1) or plasminogen activator inhibitor 2 (PAI-2), the Von Willebrand factor, or an α-1-proteinase inhibitor, ii) a erythrocyte or thrombocyte protein, e.g., hemoglobin, thrombospondin or platelet factor 4, iii) a cytokine, e.g., an interleukin such as IL-1 (e.g., IL-1α or IL-1β), IL-2, IL-4, IL-5, IL-6, IL-9, IL-10, IL-1 1, IL-12, IL-13, IL-15, IL-16, IL-17, IL-18, IL-19, IL-20, IL-21, IL-22, IL-23, a cytokine-related polypeptide, such as IL-1Ra, an interferon such as interferon-α, interferon-β or interferon-γ, a colony-stimulating factor such as GM-CSF or G-CSF, stem cell factor (SCF), a binding protein, a member of the tumor necrosis factor family (e.g TNF-α, lymphotoxin-α, lymphotoxin-β, FasL, CD40L, CD30L, CD27L, Ox40L, 4-1BBL, RANKL, TRAIL, TWEAK, LIGHT, TRANCE, APRIL, THANK or TALL-1), iv) a growth factor, e.g platelet-derived growth factor (PDGF), transforming growth factor α (TGF-α), transforming growth factor β (TGF-β), epidermal growth factor (EGF), vascular endothelial growth factor (VEGF), somatotropin (growth hormone), a somatomedin such as insulin-like growth factor I (IGF-I) or insulin-like growth factor II (IGF-II), erythropoietin (EPO), thrombopoietin (TPO) or angiopoietin, v) a profibrinolytic protein, e.g., staphylokinase or streptokinase, vi) a protease inhibitor, e.g., aprotinin or CI-2A, vii) an enzyme, e.g., superoxide dismutase, catalase, uricase, bilirubin oxidase, trypsin, papain, asparaginase, arginase, arginine deiminase, adenosin deaminase, ribonuclease, alkaline phosphatase, β-glucuronidase, purine nucleoside phosphorylase or batroxobin, viii) an opioid, e.g., endorphins, enkephalins or non-natural opioids, ix) a hormone or neuropeptide, e.g., insulin, calcitonin, glucagons, adrenocorticotropic hormone (ACTH), somatostatin, gastrins, cholecystokinins, parathyroid hormone (PTH), luteinizing hormone (LH), follicle-stimulating hormone (FSH), gonadofropin-releasing hormone, chorionic gonadotropin, corticotropin-releasing factor, vasopressin, oxytocin, antidiuretic hormones, thyroid-stimulating hormone, thyrotropin-releasing hormone, relaxin, glucagon-like peptide 1 (GLP-1), glucagon-like peptide 2 (GLP-2), prolactin, neuropeptide Y, peptide YY, pancreatic polypeptide, leptin, orexin, CART (cocaine and amphetamine regulated transcript), a CART-related peptide, melanocortins (melanocyte-stimulating hormones), melanin-concentrating hormone, natriuretic peptides, adrenomedullin, endothelin, exendin, secretin, amylin (IAPP;islet amyloid polypeptide precursor), vasoactive intestinal peptide (VIP), pituitary adenylate cyclase activating polypeptide (PACAP), agouti and agouti-related peptides or somatotropin-releasing hormones, or x) another type of protein or peptide such as thymosin, bombesin, bombesin-like peptides, heparin-binding protein, soluble CD4, pigmentary hormones, hypothalamic releasing factor, malanotonins, phospholipase activating protein, a detoxifying enzyme such as acyloxyacyl hydrolase, or an antimicrobial peptide.

One group of polypeptides of particular interest in the present invention is selected from the group of lysosomal enzymes (as defined in U.S. Pat. No. 5,929,304) such as those responsible for or otherwise involved in a lysosomal storage disease, i.e., enzymes that have a therapeutical effect on patients with a lysosomal storage disease. Such enzymes, e.g., include glucocerebrosidase, α-L-iduronidase, acid α-glucosidase, α-galactosidase, acid sphingomyelinase, galactocerebrosidase, arylsulphatase A, sialidase, and hexosaminidase. Also, other proteins involved in lysosomal storage diseases such as Saposin A, B, C or D (Nakano et al., J. Biochem. (Tokyo) 105, 152-154, 1989; Gavrieli-Rorman and Grabowski, Genomics 5, 486492, 1989) can be modified as described herein. Preferably, these polypeptides are of human origin.

The present inventors have shown that providing such enzymes with additional N-linked oligosaccharide moieties considerably improve properties thereof, such as stability, targeting, expression, and in vivo activity and targeting. Accordingly, in one embodiment, the polypeptide of the invention is a glycosylated lysosomal enzyme comprising a peptide addition comprising or contributing to a glycosylation site.

The industrial polypeptide is typically an enzyme, in particular a microbial enzyme, and can be used in products or in the manufacture of products such as detergents, household articles, personal care products, agrochemicals, textile, food products, in particular bakery products, feed products, or in industrial processes such as hard surface cleaning. The industrial polypeptide is normally not intended for internal administration to humans or animals. Specific examples include hydrolases, such as proteases, lipases or cutinases, oxidoreductases, such as laccase and peroxidase, transferases such as transglutaminases, isomerases, such as protein disulphide isomerase and glucose isomerase, cell wall degrading enzymes such as cellulases, xylanases, pectinases, mannanases, etc., amylolytic enzymes such as endoamylases, e.g., alpha-amylases, or exo-amylases, e.g., beta-amylases or amyloglucosidases, etc. Further specific examples are those listed in WO 00/26354, the contents of which are incorporated herein by reference. Normally, an enzyme modified according to the present invention has one or more improved properties selected from the group consisting of increased stability (in particular against proteolytic degradation or thermal degradation) leading to, e.g., improved shelf life and improved performance in use; improved production, e.g., in terms of improved expression (e.g., as a consequence of improved secretion and/or increased stability of the expressed enzyme) and improved purification, decreased allergenicity, increased activity in the relevant industrial process in which it is used, and improved properties with respect to immobilization.

When the polypeptide Pp is an industrial enzyme the N-terminal peptide addition may comprise or contribute to a glycosylation site. However, it is also within the scope of the present invention to provide a polypeptide comprising an industrial enzyme and a C-terminal or N-terminal peptide addition comprising an attachment group for a second non-peptide moiety being a polymer, e.g., PEG. The peptide addition may or may not comprise a glycosylation site. The peptide addition is preferably as described herein. For instance, such attachment group can be provided by a lysine or cysteine residue.

In one embodiment, the polypeptide of the invention comprises a personal care enzyme (i.e., an enzyme useful for personal care applications), which polypeptide is incapable of passing the mucous membrane of a mammal, in particular a human exposed to the polypeptide. Thereby, allergenicity can be reduced or avoided. Furthermore, stability of such enzyme can be increased. The polypeptide according to this embodiment comprises an N-terminal or C-terminal peptide addition comprising or contributing to a glycosylation site and/or an attachment group for a second non-peptide moeity, e.g., a polymer such as PEG.

In another embodiment, the polypeptide comprises a lipase as disclosed in WO 97/04079, in particular a Humicola lanuginosa lipase, wherein the N- or C-terminal peptide addition comprises a glycosylation site and/or at least one attachment group for a second non-peptide moeity, e.g., a polymer such as PEG. Thereby, the N- or C-terminal peptide addition is shielded from degradation and/or increased expression, including secretion, of the enzyme is likely to be obtained. In connection with this embodiment, the N-terminal peptide addition can comprise any of the peptide additions disclosed in WO 97/04079.

In yet another embodiment, the polypeptide Pp is an amyloglucosidase and the N- or C-terminal peptide addition comprises or contributes to a glycosylation site and/or an attachment group for a second non-peptide moeity, e.g., a polymer such as PEG. When the peptide addition is N-terminal the modification of such enzyme is contemplated to result in reduced or no degradation of the N-terminus of said enzyme (an otherwise well known problem associated with the recombinant production of amyloglucosidase). In other words, the N-terminus of the enzyme is protected by the non-peptide moiety attached to the N-terminal peptide addition of the amyloglucosidase.

In yet another embodiment, the polypeptide Pp is an antigen, in particular an antigen intended for use in eliciting an immune response (for vaccine purposes). It is contemplated to be advantageous to add N-terminal glycosylation site(s) to antigens in accordance with the invention in that the risk of changing antigenicity is thereby reduced. Antigens are recognized by a wide range of target cells, including antigen presenting cells (APC), and taken up by those cells for efficient intracellular processing and presentation to other cells of the immune system, such as, e.g., T cells, to induce or elicit desired immune responses. Antigens (and fragments thereof, e.g., antigen peptides) can be modified by a peptide addition and non-peptide moieties according to the invention. Such modifications facilitate and/or optimize uptake and/or targeting to processing compartment of the antigen by such target cells. For example, N-terminally extended antigen polypeptides of the invention are taken up by the target cells more efficiently and/or at an enhanced or improved rate (when the non-peptide moiety is one involved in such uptake). Such efficient, improved, or enhanced uptake of modified antigens by the target cells increases the kinetics and potency of the immune response to the immunizing antigen. These modifications to antigens also improve the affinity of the antigens for particular cellular receptors on target cells, including, e.g., mannose receptors and other carbohydrate receptors (in particular when the non-peptide moiety is an oligosaccharide moiety).

Antigen polypeptides of the invention include, but are not limited to those, for which an improved, enhanced or altered uptake of antigens in the following type of target cells is desired: antigen-presenting and antigen-processing cells, such as monocytes, B cells, antigen-presenting macrophages, marginal zone macrophages, follicular dendritic cells, dendritic cells, Langerhans cells, keratinocytes, M-cells (e.g., M-cells of the gut), myocytes for intramuscular immunization or epithelial cells for mucosal immunization, Kuppfer cells in the liver, and the like. A number of other cells, including capillary endothelium and some endocrine cells, can present antigen in some circumstances; the cells develop MHC class II molecules that confer antigen-presenting function. Furthermore, MHC class I molecules are expressed on the surface of most nucleated cells, including, for example, muscle cells, and therefore these cells can also present antigens to CD8+ T cells. Activated T cells, which release IFN-gamma actively induce expression of MHC molecules on some tissue cells. Such cells are also of use with the novel polypeptides of the invention. Preferably, such cells are of mammalian origin, in particular human (for use in immunization of a human) or animal (for veterinary purposes).

A wide range of antigens can be modified according to the invention. Examples are as follows:

Cancer Antigens

Examples of cancer antigens that can be modified according to the invention include, but are not limited to: bullous pemphigoid antigen 2, prostate mucin antigen (PMA) (Beckett and Wright (1995) Int. J. Cancer 62: 703-710), tumor associated Thomsen-Friedenreich antigen (Dahlenborg et al. (1997) Int. J. Cancer 70: 63-71), prostate-specific antigen (PSA) (Dannull and Belldegrun (1997) Br. J. Urol. 1: 97-103), EpCam/KSA antigen, luminal epithelial antigen (LEA. 135) of breast carcinoma and bladder transitional cell carcinoma (TCC) (Jones et al. (1997) Anticancer Res. 17: 685-687), cancer-associated serum antigen (CASA) and cancer antigen 125 (CA 125) (Kierkegaard et al. (1995) Gynecol. Oncol. 59: 251-254), the epithelial glycoprotein 40 (EGP40) (Kievit et al. (1997) Int. J. Cancer 71: 237-245), squamous cell carcinoma antigen (SCC) (Lozza et al. (1997) Anticancer Res. 17: 525-529), cathepsin E (Mota et al. (1997) Am. J. Pathol. 150: 1223-1229), tyrosinase in melanoma (Fishman et al. (1997) Cancer 79: 1461-1464), cell nuclear antigen (PCNA) of cerebral cavernomas (Notelet et al. (1997) Surg. Neurol. 47: 364-370), DF3/MUC1 breast cancer antigen (Apostolopoulos et al. (1996) Immunol. Cell. Biol. 74: 457-464; Pandey et al. (1995) Cancer Res. 55: 4000-4003), carcinoembryonic antigen (Paone et al. (1996) J. Cancer Res. Clin. Oncol. 122: 499-503; Schlom et al. (1996) Breast Cancer Res. Treat. 38: 27-39), tumor-associated antigen CA 19-9 (Tolliver and O'Brien (1997) South Med. J. 90: 89-90; Tsuruta et al. (1997) Urol. Int. 58: 20-24), human melanoma antigens MART-1/Melan-A27-35 and gp100 (Kawakami and Rosenberg (1997) Int. Rev. Immunol. 14: 173-192; Zajacetal. (1997) Int. J. Cancer 71: 491-496), theT and Tn pancarcinoma (CA) glycopeptide epitopes (Springer (1995) Crit. Rev. Oncog. 6: 57-85), a 35 kD tumor-associated autoantigen in papillary thyroid carcinoma (Lucas et al. (1996) Anticancer Res. 16: 2493-2496), KH-1 adenocarcinoma antigen (Deshpande and Danishefsky (1997) Nature 387: 164-166), the A60 mycobacterial antigen (Maes et al. (1996) J. Cancer Res. Clin. Oncol. 122: 296-300), heat shock proteins (HSPs) (Blachere and Srivastava (1995) Semin. Cancer Biol. 6: 349-355), and MAGE, tyrosinase, melan-A and gp75 and mutant oncogene products (e.g., p53, ras, and HER-2/neu (Bueler and Mulligan (1996) Mol. Med. 2: 545-555; Lewis and Houghton (1995) Semin. Cancer Biol. 6: 321-327; Theobald et al. (1995) Proc. Nat'l. Acad. Sci. USA 92: 11993-11997); TAG-72, a mucin ag expressed in most human adenocarcinomas (McGuinness et al. (1999) Hum Gene Ther 10:165-73.

Bacterial Antigens

Bacterial antigens that can be modified according to the invention include, but are not limited to, Helicobacter pylori antigens CagA and VacA (Blaser (1996) Aliment. Pharmacol. Ther. 1: 73-7; Blaser and Crabtree (1996) Am. J. Clin. Pathol. 106: 565-7; Censini et al. (1996) Proc. Nat'l. Acad. Sci. USA 93: 14648-14643). Other suitable H. pylori antigens include, for example, four immunoreactive proteins of 45-65 kDa as reported by Chatha et al. (1997) Indian J. Med. Res. 105: 170-175 and the H. pylori GroES homologue (HspA) (Kansau et al. (1996) Mol. Microbiol. 22: 1013-1023. Other suitable bacterial antigens include, but are not limited to, the 43-kDa and the fimbrilin (41 kDa) proteins of P. gingivalis (Boutsl et al. (1996) Oral Microbiol. Immunol. 11: 236-241); pneumococcal surface protein A (Briles et al. (1996) Ann. NY Acad. Sci. 797: 118-126); Chlamydia psittaci antigens, 80-90 kDa protein and 110 kDa protein (Buendia et al. (1997) FEMS Microbiol. Lett. 150: 113-9); the chlamydial exoglycolipid antigen (GLXA) (Whittum-Hudson et al. (1996) Nature Med. 2: 1116-1121); Chlamydia pneumoniae species-specific antigens in the molecular weight ranges 92-98, 51-55, 43-46 and 31.5-33 kDa and genus-specific antigens in the ranges 12, 26 and 65-70 kDa (Halme et al. (1997) Scand. J. Immunol. 45: 378-84); Neisseria gonorrhoeae (GC) or Escherichia coliu phase-variable opacity (Opa) proteins (Chen and Gotschlich (1996) Proc. Nat'l. Acad. Sci. USA 93: 14851-14856), any of the twelve immunodominant proteins of Schistosoma mansoni (ranging in molecular weight from 14 to 208 kDa) as described by Cutts and Wilson (1997) Parasitology 114: 245-55; the 17-kDa protein antigen of Brucella abortus (De Mot et al. (1996) Curr. Microbiol. 33: 26-30); a gene homolog of the 17-kDa protein antigen of the Gram-negative pathogen Brucella abortus identified in the nocardioform actinomycete Rhodococcus sp. NI86/21 (De Mot et al. (1996) Curr. Microbiol. 33: 26-30); the staphylococcal enterotoxins (SEs) (Wood et al. (1997) FEMS Immunol. Med. Microbiol. 17: 1-10), a 42-kDa M. hyopneumoniae NrdF ribonucleotide reductase R2 protein or 15-kDa subunit protein of M. hyopneumoniae (Fagan et al. (1997) Infect. Immun. 65: 2502-2507), the meningococcal antigen PorA protein (Feavers et al. (1997) Clin. Diagn. Lab. Immunol. 3: 444-50); pneumococcal surface protein A (PspA) (McDaniel et al. (1997) Gene Ther. 4: 375-377); F. tularensis outer membrane protein FopA (Fulop et al. (1996) FEMS Immunol. Med. Microbiol. 13: 245-247); the major outer membrane protein within strains of the genus Actinobacillus (Hartmann et al. (1996) Zentralbl. Bakteriol. 284: 255-262); p60 or listeriolysin (Hly) antigen of Listeria monocytogenes (Hess et al. (1996) Proc. Nat'l. Acad. Sci. USA 93: 1458-1463); flagellar (G) antigens observed on Salmonella enteritidis and S. pullorum (Holt and Chaubal (1997) J. Clin. Microbiol. 35: 1016-1020); Bacillus anthracis protective antigen (PA) (Ivins et al. (1995) Vaccine 13: 1779-1784); Echinococcus granulosus antigen 5 (Jones et al. (1996) Parasitology 1113: 213-222); the rol genes of Shigella dysenteriae 1 and Escherichia coli K-12 (Klee et al. (1997) J. Bacteriol. 179: 2421-2425); cell surface proteins Rib and alpha of group B streptococcus (Larsson et al. (1996) Infect. Immun. 64: 3518-3523); the 37 kDa secreted polypeptide encoded on the 70 kb virulence plasmid of pathogenic Yersinia spp. (Leary et al. (1995) Contrib. Microbiol. Immunol. 13: 216-217 and Roggenkamp et al. (1997) Infect. Immun. 65: 446-51); the OspA (outer surface protein A) of the Lyme disease spirochete Borrelia burgdorferi (Li et al. (1997) Proc. Nat'l. Acad. Sci. USA 94: 3584-3589, Padilla et al. (1996) J. Infect. Dis. 174: 739-746, and Wallich et al. (1996) Infection 24: 396-397); the Brucella melitensis group 3 antigen gene encoding Omp28 (Lindler et al. (1996) Infect. Immun. 64: 2490-2499); the PAc antigen of Streptococcus mutans (Murakami et al. (1997) Infect. Immun. 65: 794-797); pneumolysin, Pneumococcal neuraminidases, autolysin, hyaluronidase, and the 37 kDa pneumococcal surface adhesin A (Paton et al. (1997) Microb. Drug Resist. 3: 1-10); 29-32, 41-45, 63-71×10(3) MW antigens of Salmonella typhi (Perez et al. (1996) Immunology 89: 262-267); K-antigen as a marker of Klebsiella pneumoniae (Priamukhina and Morozova (1996) Klin. Lab. Diagn. 47-9); nocardial antigens of molecular mass approximately 60, 40, 20 and 15-10 kDa (Prokesova et al. (1996) Int. J. Immunopharmacol. 18: 661-668); Staphylococcus aureus antigen ORF-2 (Rieneck et al. (199i) Biochim Biophys Acta 1350: 128-132); GlpQ antigen of Borrelia hermsii (Schwan et al. (1996) J. Clin. Microbiol. 34: 2483-2492); cholera protective antigen (CPA) (Sciortino (1996) J. Diarrhoeal Dis. Res. 14: 16-26); a 190-kDa protein antigen of Streptococcus mutans (Senpuku et al. (1996) Oral Microbiol. Immunol. 11: 121-128); Anthrax toxin protective antigen (PA) (Sharma et al. (1996) Protein Expr. Purif. 7: 33-38); Clostridium perfringens antigens and toxoid (Strom et al. (1995) Br. J. Rheumatol. 34: 1095-1096); the SEF14 fimbrial antigen of Salmonella enteritidis (Thorns et al. (1996) Microb. Pathog. 20: 235-246); the Yersinia pestis capsular antigen (F1 antigen) (Titball et al. (1997) Infect. Immun. 65: 1926-1930); a 35-kilodalton protein of Mycobacterium leprae (Triccas et al. (1996) Infect. Immun. 64: 5171-5177); the major outer membrane protein, CD, extracted from Moraxella (Branhamella) catarrhalis (Yang et al. (1997) FEMS Immunol. Med. Microbiol. 17: 187-199); pH6 antigen (PsaA protein) of Yersinia pestis (Zav'yalov et al. (1996) FEMS Immunol. Med. Microbiol. 14: 53-57); a major surface glycoprotein. gp63, of Leishmania major (Xu and Liew (1994) Vaccine 12: 1534-1536; Xu and Liew (1995) Immunology 84: 173-176); mycobacterial heat shock protein 65, mycobacterial antigen (Mycobacterium leprae hsp65) (Lowrie et al. (1994) Vaccine 12: 1537-1540; Ragno et al. (1997) Arthritis Rheum. 40: 277-283; Silva (1995) Braz. J. Med. Biol. Res. 28: 843-851); Mycobacterium tuberculosis antigen 85 (Ag85) (Huygen et al. (1996) Nat. Med. 2: 893-898); the 45/47 kDa antigen complex (APA) of Mycobacterium tuberculosis, M. bovis and BCG (Horn et al. (1996) J. Immunol. Methods 197: 151-159); the mycobacterial antigen, 65-kDa heat shock protein, hsp65 (Tascon et al. (1996) Nat. Med. 2: 888-892); the mycobacterial antigens MPB64, MPB70, MPB57 and alpha antigen (Yamada et al. (1995) Kekkaku 70: 639-644); the M. tuberculosis 38 kDa protein (Vordermeier et al. (1995) Vaccine 13: 1576-1582); the MPT63, MPT64 and MPT-59 antigens from Mycobacterium tuberculosis (Manca et al. (1997) Infect. Immun. 65: 16-23; Oettinger et al. (1997) Scand. J. Immunol. 45: 499-503; Wilcke et al. (1996) Tuber. Lung Dis. 77: 250-256); the 35-kilodalton protein of Mycobacterium leprae (Triccas et al. (1996) Infect. Immun. 64: 5171-5177); the ESAT-6 antigen of virulent mycobacteria (Brandt et al. (1996) J. Immunol. 157: 3527-3533; Pollock and Andersen (1997) J. Infect. Dis. 175: 1251-1254); Mycobacterium tuberculosis 16-kDa antigen (Hsp16.3) (Chang et al. (1996) J. Biol. Chem. 271: 7218-7223); and the 18-kilodalton protein of Mycobacterium leprae (Baumgart et al. (1996) Infect. Immun. 64: 2274-2281); protective antigen (PA) of B. anthracis; V antigen from Yersinia pestis, Y. enterocolitica, and Y. pseudotuberculosis; antigens against bacterium Vibrio cholerae, cholera toxin B subunit, and heat-labile enterotoxins (LT) from enterotoxigenic E. coli strains.

Viral Pathogens

Polypeptides or proteins corresponding to or associated with various viral pathogens, including, but not limited to, e.g., hanta virus (e.g., hanta virus glycoproteins), flaviviruses, such as, e.g., Dengue viruses (e.g., envelope proteins), Japanese, St. Louis and Murray Valley encephalitis viruses, tick-borne encephalitis viruses can be modified according to the invention.

Viral antigens that can be modified according to the invention include, but are not limited to, influenza A virus N2 neuraminidase (Kilbourne et al. (1995) Vaccine 13: 1799-1803); Dengue virus envelope (E) and premembrane (prM) antigens (Feighny et al. (1994) Am. J. Trop. Med. Hyg. 50: 322-328; Putnak et al. (1996) Am. J. Trop. Med. Hyg. 55: 504-10); HIV antigens Gag, Pol, Vif and Nef(Vogt et al. (1995) Vaccine 13: 202-208); HIV antigens gp120 and gp160 (Achour et al. (1995) Cell. Mol. Biol. 41: 395-400; Hone et al. (1994) Dev. Biol. Stand. 82: 159-162); gp41 epitope of human immunodeficiency virus (Eckhart et al. (1996) J. Gen. Virol. 77: 2001-2008); rotavirus antigen VP4 (Mattion et al. (1995) J. Virol. 69: 5132-5137); the rotavirus protein VP7 or VP7sc (Emslie et al. (1995) J. Virol. 69: 1747-1754; Xu et al. (1995) J. Gen. Virol. 76: 1971-1980); herpes simplex virus (HSV) glycoproteins gB, gC, gD, gE, gG, gH, and gI (Fleck et al. (1994) Med. Microbiol. Immunol. (Berl) 183: 87-94 [Mattion, 1995]; Ghiasi et al. (1995) Invest. Ophthalmol. Vis. Sci. 36: 1352-1360; McLean et al. (1994) J. Infect. Dis. 170: 1100-1109); immediate-early protein ICP47 of herpes simplex virus-type 1 (HSV-1) (Banks et al. (1994) Virology 200: 236-245); immediate-early (1E) proteins ICP27, ICPO, and ICP4 of herpes simplex virus (Manickan et al. (1995) J. Virol. 69: 4711-4716); influenza virus nucleoprotein and hemagglutinin (Deck et al. (1997) Vaccine 15: 71-78; Fu et al. (1997) J. Virol. 71: 2715-2721); B19 parvovirus capsid proteins VP1 (Kawase et al. (1995) Virology 211: 359-366) or VP2 (Brown et al. (1994) Virology 198: 477-488); Hepatitis B virus core and e antigen and capsid protein (Schodel et al. (1996) Intervirology 39: 104-106); hepatitis B surface antigen (Shiau and Murray (1997) J. Med. Virol. 51: 159-166); hepatitis B surface antigen fused to the core antigen of the virus (Id.); Hepatitis B virus core-preS2 particles (Nemeckova et al. (1996) Acta Virol. 40: 273-279); HBV preS2-S protein (Kutinova et al. (1996) Vaccine 14: 1045-1052); VZV glycoprotein I (Kutinova et al. (1996) Vaccine 14: 1045-1052); rabies virus glycoproteins (Xiang et al. (1994) Virology 199: 132-140; Xuan et al. (1995) Virus Res. 36: 151-161) or ribonucleocapsid (Hooper et al. (1994) Proc. Nat'l. Acad. Sci. USA 91: 10908-10912); human cytomegalovirus (HCMV) glycoprotein B (UL55) (Britt et al. (1995) J. Infect. Dis. 171: 18-25); the hepatitis C virus (HCV) nucleocapsid protein in a secreted or a nonsecreted form, or as a fusion protein with the middle (pre-S2 and S) cr major (S) surface antigens of hepatitis B virus (HBV) (Inchauspe et al. (1997) DNA Cell Biol. 16: 185-195; Major et al. (1995) J. Virol. 69: 5798-5805); the hepatitis C virus antigens: the core protein (pC); E1 (pE1) and E2 (pE2) alone or as fusion proteins (Saito et al. (1997) Gastroenterology 112: 1321-1330); the gene encoding respiratory syncytial virus fusion protein (PFP-2) (Falsey and Walsh (1996) Vaccine 14: 1214-1218; Piedra et al. (1996) Pediatr. Infect. Dis. J 15: 23-31); the VP6 and VP7 genes of rotaviruses (Choi et al. (1997) Virology 232: 129-138; Jin et al. (1996) Arch. Virol. 141: 2057-2076); the E1, E2, E3, E4, E5, E6 and E7 proteins of human papillomavirus (Brown et al. (1994) Virology 201: 46-54; Dillner et al. (1995) Cancer Detect. Prev. 19: 381-393; Krul et al. (1996) Cancer Immunol. Immunother. 43: 44-48; Nakagawa et al., (1997) J. Infect. Dis. 175: 927-931); a human T-lymphotropic virus type I gag protein (Porter et al. (1995) J. Med. Virol. 45: 469-474); Epstein-Barr virus (EBV) gp340 (Mackett et al. (1996) J. Med. Virol. 50: 263-271); the Epstein-Barr virus (EBV) latent membrane protein LMP2 (Lee et al. (1996) Eur. J. Immunol. 26: 1875-1883); Epstein-Barr virus nuclear antigens 1 and 2 (Chen and Cooper (1996) J. Virol. 70: 4849-4853; Khanna et al. (1995) Virology 214: 633-637); the measles virus nucleoprotein (N) (Fooks et al. (1995) Virology 210: 456-465); and cytomegalovirus glycoprotein gB (Marshall et al. (1994) J. Med. Virol. 43: 77-83) or glycoprotein gH (Rasmussen et al. (1994) J. Infect. Dis. 170: 673-677).

Parasites

Antigens from parasites can also be modified according to the invention. These include, but are not limited to, the schistosome gut-associated antigens CAA (circulating anodic antigen) and CCA (circulating cathodic antigen) in Schistosoma mansoni, S. haematobium or S. japonicum (Deelder et al. (1996) Parasitology 112: 21-35); a multiple antigen peptide (MAP) composed of two distinct protective antigens derived from the parasite Schistosoma mansoni (Ferru et al. (1997) Parasite Immunol. 19: 1-11); Leishmania parasite surface molecules (Lezama-Davila (1997) Arch. Med. Res. 28: 47-53); third-stage larval (L3) antigens of L. loa (Akue et al. (1997) J. Infect. Dis. 175: 158-63); the genes, Tams1-1 and Tams1-2, encoding the 30-and 32-kDa major merozoite surface antigens of Theileria annulata (Ta) (d'Oliveira et al. (1996) Gene 172: 33-39); Plasmodium falciparum merozoite surface antigen 1 or 2 (al-Yaman et al. (1995) Trans. R. Soc. Trop. Med. Hyg. 89: 555-559; Beck et al. (1997) J. Infect. Dis. 175: 921-926; Rzepczyk et al. (1997) Infect. Immun. 65: 1098-1100); circumsporozoite (CS) protein-based B-epitopes from Plasmodium berghei, (PPPPNPND)₂ and Plasmodium yoelii, (QGPGAP)3QG, along with a P. berghei T-helper epitope KQIRDSITEEWS (Reed et al. (1997) Vaccine 15: 482-488); NYVAC-Pf7 encoded Plasmodium falciparum antigens derived from the sporozoite (circumsporozoite protein and sporozoite surface protein 2), liver (liver stage antigen 1), blood (merozoite surface protein 1, serine repeat antigen, and apical membrane antigen 1), and sexual (25-kDa sexual-stage antigen) stages of the parasite life cycle were inserted into a single NYVAC genome to generate NYVAC-Pf7 (Tine et al. (1996) Infect. Immun. 64: 3833-3844); Plasmodium falciparum antigen Pfs230 (Williamson et al. (1996) Mol. Biochem. Parasitol. 78: 161-169); Plasmodium falciparum apical membrane antigen (AMA-1) (Lal et al. (1996) Infect. Immun. 64: 1054-1059); Plasmodium falciparum proteins Pfs28 and Pfs25 (Duffy and Kaslow (1997) Infect. Immun. 65: 1109-1113); Plasmodium falciparum merozoite surface protein, MSP1 (Hui et al. (1996) Infect. Immun. 64: 1502-1509); the malaria antigen Pf332 (Ahlborg et al. (1996) Immunology 88: 630-635); Plasmodium falciparum erythrocyte membrane protein 1 (Baruch et al. (1995) Proc. Nat'l. Acad. Sci. USA 93: 3497-3502; Baruch et al. (1995) Cell 82: 77-87); Plasmodium falciparum merozoite surface antigen, PfMSP-1 (Egan et al. (1996) J. Infect. Dis. 173: 765-769); Plasmodium falciparum antigens SERA, EBA-175, RAP1 and RAP2 (Riley (1997) J. Pharm. Pharmacol. 49: 21-27); Schistosoma japonicum paramyosin (Sj97) or fragments thereof (Yang et al. (1995) Biochem. Biophys. Res. Commun. 212: 1029-1039); and Hsp70 in parasites (Maresca and Kobayashi (1994) Experientia 50: 1067-1074).

Allergen Antigens

Allergen antigens that can be modified according to the invention, include, but are not limited to those of animals, including the mite (e.g., Dermatophagoides pteronyssinus, Dermatophagoides farinae, Blomia tropicalis), such as the allergens der p1 (Scobie et al. (1994) Biochem. Soc. Trans. 22: 448S; Yssel et al. (1992) J. Immunol. 148: 738-745), der p2 (Chua et al. (1996) Clin. Exp. Allergy 26: 829-837), der p3 (Smith and Thomas (1996) Clin. Exp. Allergy 26: 571-579), der p5, der p V (Lin et al. (1994) J. Allergy Clin. Immunol. 94: 989-996), der p6 (Bennett and Thomas (1996) Clin. Exp. Allergy 26: 1150-1154), der p 7 (Shen et al. (1995) Clin. Exp. Allergy 25: 416-422), der f2 (Yuuki et al. (1997) Int. Arch. Allergy Immunol. 112: 44-48), der f3 (Nishiyama et al. (1995) FEBS Lett. 377: 62-66), der f7 (Shen et al. (1995) Clin. Exp. Allergy 25: 1000-1006); Mag 3 (Fujikawa et al. (1996) Mol. Immunol. 33: 311-319). Also of interest as antigens are the house dust mite allergens Tyr p2 (Eriksson et al. (1998) Eur. J. Biochem. 251: 443-447), Lep d1 (Schmidt et al. (1995) FEBS Lett. 370: 11-14), and glutathione S-transferase (O'Neill et al. (1995) Immunol Lett. 48: 103-107); the 25,589 Da, 219 amino acid polypeptide with homology with glutathione S-transferases (O'Neill et al. (1994) Biochim. Biophys. Acta. 1219: 521-528); Blo t 5 (Arruda et al. (1995) Int. Arch. Allergy Immunol. 107: 456-457); bee venom phospholipase A2 (Carballido et al. (1994) J. Allergy Clin. Immunol. 93: 758-767; Jutel et al. (1995) J. Immunol. 154: 4187-4194); bovine dermal/dander antigens BDA 11 (Rautiainen et al. (1995) J. Invest. Dermatol. 105: 660-663) and BDA20 (Mantyjarvi et al. (1996) J. Allergy Clin. Immunol. 97: 1297-1303); the major horse allergen Equ c1 (Gregoire et al. (1996) J. Biol. Chem. 271: 32951-32959); Jumper ant M. pilosula allergen Myr p I and its homologous allergenic polypeptides Myr p2 (Donovan el al. (1996) Biochem. Mol. Biol. Int. 39: 877-885); 1-13, 14, 16 kD allergens of the mite Blomia tropicalis (Caraballo et al. (1996) J. Allergy Clin. Immunol. 98: 573-579); the cockroach allergens Bla g Bd90K (Helm et al. (1996) J. Allergy Clin. Immunol. 98: 172-80) and Bla g 2 (Armida et al. (1995) J. Biol. Chem. 270: 19563-19568); the cockroach Cr-PI allergens (Wu et al. (1996) J. Biol. Chem. 271: 17937-17943); fire ant venom allergen, Sol i 2 (Schmidt et al. (1996) J. Allergy Clin. Immunol. 98: 82-88); the insect Chironomus thummi major allergen Chi t 1-9 (Kipp et al. (1996) Int. Arch. Allergy Immunol. 110: 348-353); dog allergen Can f I or cat allergen Fel d I (Ingram et al. (1995) J. Allergy Clin. Immunol. 96: 449-456); albumin, derived, for example, from horse, dog or cat (Goubran Botros et al. (1996) Immunology 88: 340-347); deer allergens with the molecular mass of 22 kD, 25 kD or 60 kD (Spitzauer et al. (1997) Clin. Exp. Allergy 27: 196-200); and the 20 kd major allergen of cow (Ylonen et al. (1994) J. Allergy Clin. Immunol. 93: 851-858).

Pollen and grass allergens can also be modified according to the invention. Such allergens include, for example, Hor v9 (Astwood and Hill (1996) Gene 182: 53-62, Lig v1 (Batanero et al. (1996) Clin. Exp. Allergy 26: 1401-1410); Lol p 1 (Muller et al. (1996) Int. Arch. Allergy Immunol. 109: 352-355), Lol p 11 (Tamborini et al. (1995) Mol. Immunol. 32: 505-513), Lol pVA, Lol pVB (Ong et al. (1995) Mol. Immunol. 32: 295-302), Lol p 9 (Blaher et al. (1996) J. Allergy Clin. Immunol. 98: 124-132); Par J I (Costa et al. (1994) FEBS Lett. 341: 182-186; Sallusto et al. (1996) J. Allergy Clin. Immunol. 97: 627-637), Par j 2.0101 (Duro et al. (1996) FEBS Lett. 399: 295-298); Bet v1 (Faber et al. (1996) J. Biol. Chem. 271: 19243-19250), Bet v2 (Rihs et al. (1994) Int. Arch. Allergy Immunol. 105: 190-194); Dac g3 (Guerin-Marchand et al. (1996) Mol. Immunol. 33: 797-806); Phl p 1 (Petersen et al. (1995) J. Allergy Clin. Immunol. 95: 987-994), Phl p 5 (Muller et al. (1996) Int. Arch. Allergy Immunol. 109: 352-355), Phl p 6 (Petersen et al. (1995) Int. Arch. Allergy Immunol. 108: 55-59); Cry j I (Sone et al. (1994) Biochem. Biophys. Res. Commun. 199: 619-625), Cry j II (Namba et al. (1994) FEBS Lett. 353: 124-128); Cor a 1 (Schenk et al. (1994) Eur. J. Biochem. 224: 717-722); cyn d1 (Smith et al. (1996) J. Allergy Clin. Immunol. 98: 331-343), cyn d7 (Suphioglu et al. (1997) FEBS Lett. 402: 167-172); Pha a I and isoforms of Pha a 5 (Suphioglu and Singh (1995) Clin. Exp. Allergy 25: 853-865); Cha o 1 (Suzuki et al. (1996) Mol. Immunol. 33: 451-460); profilin derived, i.e., from timothy grass or birch pollen (Valenta et al. (1994) Biochem. Biophys. Res. Commun. 199: 106-118); P0149 (Wu et al. (1996) Plant Mol. Biol. 32: 1037-1042); Ory s1 (Xu et al. (1995) Gene 164: 255-259); and Amb a V and Amb t 5 (Kim et al. (1996) Mol. Immunol. 33: 873-880; Zhu et al. (1995) J. Immunol. 155: 5064-5073).

Food allergens that can be modified according to the invention include, for example, profilin (Rihs et al. (1994) Int. Arch. Allergy Immunol. 105: 190-194); rice allergenic cDNAs belonging to the alpha-amylase/trypsin inhibitor gene family (Alvarez et al. (1995) Biochim Biophys Acta 1251: 201-204); the main olive allergen, Ole e I (Lombardero et al. (1994) Clin Exp Allergy 24: 765-770); Sin a 1, the major allergen from mustard (Gonzalez De La Pena et al. (1996) Eur J. Biochem. 237: 827-832); parvalbumin, the major allergen of salmon (Lindstrom et al. (1996) Scand. J. Immunol. 44: 335-344); apple allergens, such as the major allergen Mal d I (Vanek-Krebitz et al. (1995) Biochem. Biophys. Res. Commun. 214: 538-551); and peanut allergens, such as Ara h I (Burks et al. (1995) J. Clin. Invest. 96: 1715-1721).

Fungal allergens that can be modified according to the invention include, but are not limited to, the allergen, Cla h III, of Cladosporium herbarum (Zhang et al. (1995) J. Immunol. 154: 710-717); the allergen Psi c 2, a fungal cyclophilin, from the basidiomycete Psilocybe cubensis (Homer et al. (1995) Int. Arch. Allergy Immunol. 107: 298-300); hsp 70 cloned from a cDNA library of Cladosporium herbarum (Zhang et al. (1996) Clin Exp Allergy 26: 88-95); the 68 kD allergen of Penicillium notatum (Shen et al. (1995) Clin. Exp. Allergy 26: 350-356); aldehyde dehydrogenase (ALDH) (Achatz et al. (1995) Mol Immunol. 32: 213-227); enolase (Achatz et al. (1995) Mol. Immunol. 32: 213-227); YCP4 (Id.); acidic ribosomal protein P2 (Id.).

Other allergens that can be modified include latex allergens, such as a major allergen (Hev b 5) from natural rubber latex (Akasawa et al. (1996) J. Biol. Chem. 271: 25389-25393; Slater et al. (1996) J. Biol. Chem. 271: 25394-25399).

Antigens Associated with Autoimmune Diseases and Inflammatory Conditions

Autoantigens that can be modified according to the invention include, but are not limited to, myelin basic protein (Stinissen et al. (1996) J. Neurosci. Res. 45: 500-511) or a fusion protein of myelin basic protein and proteolipid protein (Elliott et al. (1996) J. Clin. Invest. 98: 1602-1612), proteolipid protein (PLP) (Rosener et al. (1997) J. Neuroimmunol. 75: 28-34), 2′,3′-cyclic nucleotide 3′-phosphodiesterase (CNPase) (Rosener et al. (1997) J Neuroimmunol. 75: 28-34), the Epstein Barr virus nuclear antigen-1 (EBNA-1) (Vaughan et al. (1996) J. Neuroimmunol. 69: 95-102), HSP70 (Salvetti et al. (1996) J. Neuroimmunol. 65: 143-53; Feldmann et al. (1996) Cell 85: 307).

Antigens that can be modified according to the invention and used to treat scleroderma, systemic sclerosis, and systemic lupus erythematosus include, for example, (−2-GPI, 50 kDa glycoprotein (Blank et al. (1994) J. Autoimmun. 7: 441-455), Ku (p70/p80) autoantigen, or its 80-kd subunit protein (Hong et al. (1994) Invest. Ophthalmol. Vis. Sci. 35: 4023-4030; Wang et al. (1994) J. Cell Sci. 107: 3223-3233), the nuclear autoantigens La (SS-B) and Ro (SS-A) (Huang et al. (1997) J. Clin. Immunol. 17: 212-219; Igarashi et al. (1995) Autoimmunity 22: 33-42; Keech et al. (1996) Clin. Exp. Immunol. 104: 255-263; Manoussakis et al. (1995) J. Autoimmun. 8: 959-969; Topfer et al. (1995) Proc. Nat'l. Acad. Sci. USA 92: 875-879), proteasome (-type subunit C9 (Feist et al. (1996) J. Exp. Med. 184: 1313-1318), Scleroderma antigens Rpp 30, Rpp 38 or Scl-70 (Eder et al. (1997) Proc. Nat'l. Acad. Sci. USA 94: 1101-1106; Hietarinta et al. (1994) Br. J. Rheumatol. 33: 323-326), the centrosome autoantigen PCM-1 (Bao et al. (1995) Autoimmunity 22: 219-228), polymyositis-scleroderma autoantigen (PM-Scl) (Kho et al. (1997) J. Biol. Chem. 272: 13426-13431), scleroderma (and other systemic autoimmune disease) autoantigen CENP-A (Muro et al. (1996) Clin. Immunol. Immunopathol. 78: 86-89), U5, a small nuclear ribonucleoprotein (snRNR) (Okano et al. (1996) Clin. Immunol. Immunopathol. 81: 41-47), the 100-kd protein of PM-Scl autoantigen (Ge et al. (1996) Arthritis Rheum. 39: 1588-15955), the nucleolar U3- and Th(7-2) ribonucleoproteins (Verheijen et al. (1994) J. Immunol. Methods 169: 173-182), the ribosomal protein L7 (Neu et al. (1995) Clin. Exp. Immunol. 100: 198-204), hPop1 (Lygerou et al. (1996) EMBO J. 15: 5936-5948), and a 36-kd protein from nuclear matrix antigen (Deng et al. (1996) Arthritis Rheum. 39: 1300-1307).

Antigens useful in treatment of hepatic autoimmune disorders can also be modified; these include the cytochromes P450 and UDP-glucuronosyl-transferases (Obennayer-Straub and Manns (1996) Baillieres Clin. Gastroenterol. 10: 501-532), the cytochromes P450 2C9 and P450 1A2 (Bourdi et al. (1996) Chem. Res. Toxicol. 9: 1159-1166; Clemente et al. (1997) J. Clin. Endocrinol. Metab. 82: 1353-1361), LC-1 antigen (Klein et al. (1996) J. Pediatr. Gastroenterol. Nutr. 23: 461-465), and a 230-kDa Golgi-associated protein (Funaki et al. (1996) Cell Struct. Funct. 21: 63-72).

Antigens useful for treatment of autoimmune disorders of the skin that can be modified according to the invention include, but are not limited to, the 450 kD human epidermal autoantigen (Fujiwara et al. (1996) J. Invest. Dermatol. 106: 1125-1130), the 230 kD and 180 kD bullous pemphigoid antigens (Hashimoto (1995) Keio J. Med. 44: 115-123; Murakami et al. (1996) J Dermatol. Sci. 13: 112-117), pemphigus foliaceus antigen (desmoglein 1), pemphigus vulgaris antigen (desmoglein 3), BPAg2, BPAg1, and type VII collagen (Batteux et al. (1997) J. Clin. Immunol. 17: 228-233; Hashimoto et al. (1996) J. Dermatol. Sci. 12: 10-17), a 168-kDa mucosal antigen in a subset of patients with cicatricial pemphigoid (Ghohestani et al. (1996) J. Invest. Dermatol. 107: 136-139), and a 218-kd nuclear protein (218-kd Mi-2) (Seelig et al. (1995) Arthritis Rheum. 38: 1389-1399).

Antigens for treating insulin dependent diabetes mellitus can also be modified; these, include, but are not limited to, insulin, proinsulin, GAD65 and GAD67, heat-shock protein 65 (hsp65), and islet-cell antigen 69 (ICA69) (French et al. (1997) Diabetes 46: 34-39; Roep (1996) Diabetes 45: 1147-1156; Schloot et al. (1997) Diabetologia 40: 332-338), viral proteins homologous to GAD65 (Jones and Crosby (1996) Diabetologia 39: 1318-1324), islet cell antigen-related protein-tyrosine phosphatase (PTP) (Cui et al. (1996) J. Biol. Chem. 271: 24817-24823), GM2-1 ganglioside (Cavallo et al. (1996) J. Endocrinol. 150: 113-120; Dotta et al. (1996) Diabetes 45: 1193-1196), glutamic acid decarboxylase (GAD) (Nepom (1995) Curr. Opin. Immunol. 7: 825-830; Panina-Bordignon et al. (1995) J. Exp. Med. 181: 1923-1927), an islet cell antigen (ICA69) (Karges et al. (1997) Biochim. Biophys. Acta 1360: 97-101; Roep et al. (1996) Eur. J. Immunol. 26: 1285-1289), Tep69, the single T cell epitope recognized by T cells from diabetes patients (Karges et al. (1997) Biochim. Biophys. Acta 1360: 97-101), ICA 512, an autoantigen of type I diabetes (Solimena et al. (1996) EMBO J. 15: 2102-2114), an islet-cell protein tyrosine phosphatase and the 37-kDa autoantigen derived from it in type 1 diabetes (including IA-2, IA-2) (La Gasse et al. (1997) Mol. Med. 3: 163-173), the 64 kDa protein from In-111 cells or human thyroid follicular cells that is immunoprecipitated with sera from patients with islet cell surface antibodies (ICSA) (Igawa et al. (1996) Endocr. J. 43: 299-306), phogrin, a homologue of the human transmembrane protein tyrosine phosphatase, an autoantigen of type 1 diabetes (Kawasaki et al. (1996) Biochem. Biophys. Res. Commun. 227: 440-447), the 40 kDa and 37 kDa tryptic fragments and their precursors IA-2 and IA-2 in IDDM (Lampasona et al. (1996) J. Immunol. 157: 2707-2711; Notkins et al. (1996) J. Autoimmun. 9: 677-682), insulin or a cholera toxoid-insulin polypeptide (Bergerot et al. (1997) Proc. Nat'l. Acad. Sci. USA 94: 4610-4614), carboxypeptidase H, the human homologue of gp330, which is a renal epithelial glycoprotein involved in inducing Heymann nephritis in rats, and the 38-kD islet mitochondrial autoantigen (Arden et al. (1996) J. Clin. Invest. 97: 551-561.

Useful antigens for rheumatoid arthritis treatment that can be modified according to the invention include, but are not limited to, the 45 kDa DEK nuclear antigen, in particular onset juvenile rheumatoid arthritis and iridocyclitis (Murray et al. (1997) J. Rheumatol. 24: 560-567), human cartilage glycoprotein-39, an autoantigen in rheumatoid arthritis (Verheijden et al. (1997) Arthritis Rheum. 40: 1115-1125), a 68k autoantigen in rheumatoid arthritis (Blass et al. (1997) Ann. Rheum. Dis. 56: 317-322), collagen (Rosloniec et al. (1995) J. Immunol. 155: 4504-4511), collagen type II (Cook et al. (1996) Arthritis Rheum. 39: 1720-1727; Trentham (1996) Ann. N.Y. Acad. Sci. 778: 306-314), cartilage link protein (Guerassimov et al. (1997) J. Rheumatol. 24: 959-964), ezrin, radixin and moesin, which are auto-immune antigens in rheumatoid arthritis (Wagatsuma et al. (1996) Mol. Immunol. 33: 1171-1176), and mycobacterial heat shock protein 65 (Ragno et al. (1997) Arthritis Rheum. 40: 277-283).

Antigens useful for treatment are autoimmune thyroid disorders that can be modified include, for example, thyroid peroxidase and the thyroid stimulating hormone receptor (Tandon and Weetman (1994) J. R. Coll. Physicians Lond. 28: 10-18), thyroid peroxidase from human Grave3′ thyroid tissue (Gardas et al. (1997) Biochem. Biophys. Res. Commun. 234: 366-370; Zimmer et al. (1997) Histochem. Cell. Biol. 107: 115-120), a 64-kDa antigen associated with thyroid-associated ophthalmopathy (Zhang et al. (1996) Clin. Immunol. Immunopathol. 80: 236-244), the human TSH receptor (Nicholson et al. (1996) J. Mol. Endocrinol. 16: 159-170), and the 64 kDa protein from In-111 cells or human thyroid follicular cells that is immunoprecipitated with sera from patients with islet cell surface antibodies (ICSA) (Igawa et al. (1996) Endocr. J. 43: 299-306).

Other associated antigens that can be modified include, but are not limited to, Sjogren's syndrome (-fodrin; Haneji et al. (1997) Science 276: 604-607), myastenia gravis (the human M2 acetylcholine receptor or fragments thereof, specifically the second extracellular loop of the human M2 acetylcholine receptor; Fu et al. (1996) Clin. Immunol. Immunopathol. 78: 203-207), vitiligo (tyrosinase; Fishman et al. (1997) Cancer 79: 1461-1464), a 450 kD human epidermal autoantigen recognized by serum from individual with blistering skin disease, and ulcerative colitis (chromosomal proteins HMG1 and HMG2; Sobajima et al. (1997) Clin. Exp. Immunol. 107: 135-140).

Sperm Antigens

Sperm antigens which can be used in the genetic vaccines include, for example, lactate dehydrogenase (LDH-C4), galactosyltransferase (GT), SP-10, rabbit sperm autoantigen (RSA), guinea pig (g)PH-20, cleavage signal protein (CS-1), HSA-63, human (h)PH-20, and AgX-1 (Zhu and Naz (1994) Arch. Androl. 33: 141-144), the synthetic sperm peptide, P10G (O'Rand et al. (1993) J. Reprod. Immunol. 25: 89-102), the 135 kD, 95 kD, 65 kD, 47 kD, 41 kD and 23 kD proteins of sperm, and the FA-1 antigen (Naz et al. (1995) Arch. Androl. 35: 225-231), and the 35 kD fragment of cytokeratin 1 (Lucas et al. (1996) Anticancer Res. 16: 2493-2496).

Also, examples of antigens are set forth in Punnonen et al. (1999) WO 99/41369; Punnonen et al. (1999) WO 99/41383; Punnonen et al. (1999) WO 99/41368; and Punnonen et al. (1999) WO 99/41402), the contents of all of which are incorporated herein by reference in their entirety for all purposes. Other useful antigens have been described in the literature or can be discovered using genomics approaches.

Peptide Addition

In principle the peptide addition X can be any stretch of amino acid residues ranging from a single amino acid residue to a large protein, e.g., a mature protein. Usually, the peptide addition X comprises 1-500 amino acid residues, such as 2-500, normally 2-50 or 3-50 amino acid residues, such as 3-20 amino acid residues. The length of the peptide addition to be used for modification of a given polypeptide is dependent of or determined on the basis of a number of factors including the type of polypeptide of interest and the desired effect to be achieved by the modification. Normally, the peptide addition has less than 90% identity to the amino acid sequence of a native full length polypeptide, in particular less than 80% identity, such as less than 70% identity or even lower degree of identity to a full length protein. In one embodiment, the peptide addition may constitute a part of a full length protein (e.g., 1-50 amino acid residues thereof.

The peptide addition may be designed by a site-specific or random approach, e.g as out-lined in further detail in the Methods section below. This section also comprises a set of guidelines useful for preparing a peptide addition for use in the present invention are described. It will be understood that those guidelines are intended for illustration purposes only and that a person skilled in the art will be aware of alternative useful routes for design of peptide addition. Thus, the method of designing a peptide addition for use herein should not be considered limited to that described in the Materials section.

The number of glycosylation sites should be sufficient to provide the desired effect. Typically, the peptide addition X comprises 1-20, such as 1-10 glycosylation sites. For instance, the peptide addition X comprises 1, 2, 3, 4, 5, 6, 7, 8, 9 or 10 glycosylation sites. It is well known that one frequently occurring consequence of modifying an amino acid sequence of, e.g., a human protein is that new epitopes are created by such modification. In order to shield any new epitopes created by the peptide addition, it is desirable that sufficient glycosylation sites are present to enable shielding of all epitopes introduced into the sequence. This is e.g., achieved when the peptide addition X comprises at least one glycosylation site within a stretch of 30 contiguous amino acid residues, such as at least one glycosylation site within 20 amino acid residues or at least one glycosylation site within 10 amino acid residues, in particular 1-3 glycosylation sites within a stretch of 10 contiguous amino acid residues in the peptide addition X.

Thus, in one embodiment, the peptide addition X comprises at least two glycosylation sites, wherein two of said sites are separated by at most 10 amino acid residues, none of which comprises a glycosylation site. Furthermore, the polypeptide Pp can comprise at least one introduced glycosylation site, in particular 1-5 introduced glycosylation sites. Analogously, the polypeptide Pp can comprise at least one removed glycosylation site, in particular 1-5 removed glycosylation sites.

The glycosylation site of the peptide addition may be an in vivo or in vitro glycosylation site. Prefererably, the glycosylation site is an in vivo glycosylation site, in particular an N-glycosylation site since glycosylation of such site is more easy to control than to an O-glycosylation site. Accordingly, in an embodiment, the peptide addition X comprises at least one N-glycosylation site, typically at least two N-glycosylation sites. For instance, the peptide addition X has the structure X₁-N-X₂-[T/S]/C-Z, wherein X₁ is a peptide comprising at least one amino acid residue or is absent, X₂ is any amino acid residue different from Pro, and Z is absent or a peptide comprising at least one amino acid residue. For instance, X₁ is absent, X₂ is an amino acid residue selected from the group consisting of I, A, G, V and S (all relatively small amino acid residues), and Z comprises at least I amino acid residue.

For instance, Z can be a peptide comprising 1-50 amino acid residues and, e.g., 1-10 glycosylation sites.

In another polypeptide of the invention X₁ comprises at least one amino acid residue, e.g., 1-50 amino acid residues, X₂ is an amino acid residue selected from the group consisting of I, A, G, V and S, and Z is absent. For instance, X₁ comprises 1-10 glycosylation sites.

For instance, the peptide addition for use in the present invention can comprise a peptide sequence selected from the group consisting of INA[T/S], GNI[T/S], VNI[T/S], SNI[T/S], ASNI[T/S], NI[T/S], SPINA[T/S], ASPINA[T/S], ANI[T/S]ANI[T/S]ANI, ANI[T/S]GSNI[T/S]GSNI[T/S], FNI[T/S]VNI[T/S]V, YNI[T/S]VNI[T/S]V, AFNI[T/S]VNI[T/S]V, AYNI[T/S]VNI[T/S]V, APND[T/S]VNI[T/S]V, ANI[T/S], ASNS[T/S]NNG[T/S]LNA[T/S], ANH[T/S]NE[T/S]NA[T/S], GSPINA[T/S], ASPINA[T/S]SPINA[T/S], ANN[T/S]NY[T/S]NW[T/S], ATNI[T/S]LNY[T/S]AN[T/S]T, AANS[T/S]GNI[T/S]ING[T/S], AVNW[T/S]SND[T/S]SNS[T/S], GNA[T/S], AVNW[T/S]SND[T/S]SNS[T/S], ANN[T/S]NY[T/S]NS[T/S], ANNTNYTNWT, ANI[T/S]VNI[T/S]V, ND[T/S]VNF[T/S] and NI[T/S]VNI[T/S]V wherein [T/S] is either a T or an S residue, preferably a T residue. Other non-limiting examples include a peptide addition comprising the sequence NSTQNATA, which corresponds to positions 231 to 238 of the human calcium activated channel 2 precursor (to add two N-glycosylation sites), or the sequence ANLTVRNLTRNVTV, which corresponds to positions 538 to 551 of the human G protein coupled receptor 64 (to add three N-glycosylation sites).

The peptide addition can comprise one or more of these peptide sequences, i.e., at least two of said sequences either directly linked together or separated by one or more amino acid residues, or can contain two or more copies of any of these peptide sequence. It will be understood that the above specific sequences are given for illustrative purposes and thus do not constitute an exclusive list of peptide sequences of use in the present invention.

In a more specific embodiment, the peptide addition X is selected from the group consisting of INA[T/S], GNI[T/S], VNI[T/S], SNI[T/S], ASNI[T/S], NI[T/S], SPINA[T/S], ASPINA[T/S], ANI[T/S]ANI[T/S]ANI, and ANI[T/S]GSNI[T/S]GSNI[T/S], wherein [T/S] is either a T or an S residue, preferably a T residue.

As stated further above the polypeptide Pp can be a native polypeptide that optionally comprises one or more glycosylation sites. In order to further modify the glycosylation of the polypeptide Pp of interest (in terms of the number of oligosaccharide moieties attached to the polypeptide), the polypeptide Pp can be a variant of a native polypeptide that differs from said polypeptide in at least one introduced or at least one removed glycosylation site.

For instance, the polypeptide Pp comprises at least one introduced glycosylation site, in particular 1-5 introduced glycosylation sites, such as 2-5 introduced glycosylation sites.

In order to affect the total glycosylation of the polypeptide of interest the glycosylation site is introduced so that the N residue of said glycosylation site is exposed at the surface of the polypeptide, when folded in its active form. Likewise, a glycosylation site to be removed is selected from those having an N residue exposed at the surface of the polypeptide.

In one embodiment, the peptide addition X has an N residue in position −2 or −1, and the polypeptide Pp or Pₓ has a T or an S residue in position +1 or +2, respectively, the residue numbering being made relative to the N-terminal amino acid residue of Pp or Pₓ, whereby an N-glycosylation site is formed.

Glycosylation

The polypeptide of the invention is glycosylated (i.e., comprises an in vivo attached N- or O-linked oligosaccharide moiety or in vitro attached oligosaccharide moiety) and furthermore has an altered glycosylation profile as compared to that of the polypeptide Pp. For instance, the altered glycosylation profile is a consequence of an altered, normally increased, number of attached oligosaccharide moieties and/or an altered type or distribution of attached oligosaccharide moieities.

Furthermore, for polypeptides intended for therapeutic or veterinary uses or to which a human or animal is otherwise exposed, the type of oligosaccharide moiety to be attached should normally be one that does not lead to increased immunogenicity of the polypeptide as compared to that of the polypeptide Pp. The coupling of an oligosaccharide moiety may take place in vivo or in vitro. In order to achieve in vivo glycosylation of a a nucleotide sequence encoding the polypeptide should be inserted in a glycosylating, eucaryotic expression host. The expression host cell may be selected from fungal (filamentous fungal or yeast), insect, mammalian cells or transgenic plant cells as disclosed in further detail in the section entitled “Methods of preparing a polypeptide of the invention”. Also, the glycosylation may be achieved in the human body when using a nucleotide sequence encoding the polypeptide of the invention in gene therapy.

In vitro glycosylation can be achieved by attaching chemically synthesized oligosaccharide structures to the polypeptide using a variety of different chemistries e.g., the chemistries employed for attachment of PEG to proteins, wherein the oligosaccharide is linked to a functional group, optionally via a short spacer (see the section entitled Conjugation to a Non-Oligosaccharide Macromolecular Moiety). The in vitro glycosylation can be carried out in a suitable buffer at pH 4-7 in protein concentrations of 0.5-2 mg/ml and a volume of 0.02-2 ml. The activated mannose compound is present in 2-200 fold molar excess, and reactions are incubated at 4-25° C. for periods of 0.1-3 hours. In vitro glycosylated GCB polypeptides are purified by dialysis and standard chromatographic techniques.

Other in vitro glycosylation methods are described, for example in WO 87/05330, by Aplin et al. (1981) CRC Crit Rev. Biochem. pp. 259-306, by Lundblad and Noyes Chemical Ragents for Protein Modification CRC Press Inc. Boca Raton, Fla., by Yan and Wold (1984) Biochemistry 23: 3759-65, and by Doebber et al. (1982) J. Biol. Chem. 257: 2193-2199.

Furthermore, in vitro glycosylation to protein- and peptide-bound Gin-residues can be carried out by transglutaminases (TGases). Transglutaminases catalyse the transfer of donor amine-groups to protein- and peptide-bound Gin-residues in a so-called cross-linking reaction. The donor-amine groups can be protein- or peptide-bound e.g., as the ε-amino-group in Lys-residues or it can be part of a small or large organic molecule. An example of a small organic molecule functioning as amino-donor in TGase-catalysed cross-linking is putrescine (1,4-diaminobutane). An example of a larger organic molecule functioning as amino-donor in TGase-catalysed cross-linking is an amine-containing PEG (Sato et al. (1996) Biochemistry 35: 13072-13080).

TGases, in general, are highly specific enzymes, and not every Gln-residues exposed on the surface of a protein is accessible to TGase-catalysed cross-linking to amino-containing substances. In order to render a protein susceptible to TGase-catalysed cross-linking reactions stretches of amino acid sequence known to function very well as TGase substrates are inserted at convenient positions in the amino acid sequence encoding a GCB polypeptide. Several amino acid sequences are known to be or to contain excellent natural TGase substrates e.g., substance P, elafin, fibrinogen, fibronectin, α₂-plasmin inhibitor, α-caseins, and β-caseins and may thus be inserted into and thereby constitute part of the amino acid sequence of a polypeptide of the invention.

The nature and number of oligosaccharide moieties of a glycosylated polypeptide of the invention may be determined by a number of different methods known in the art e.g. by lectin binding studies (Reddy et al. (1985) Biochem. Med. 33: 200-210; Cummings (1994) Meth. Enzymol. 230: 66-86; Protein Protocols (Walker ed.) (1998) chapter 9); by reagent array analysis method (RAAM) sequencing of released oligosaccharides (Edge et al. (1992) Proc. Natl. Acad. Sci. USA 89: 6338-6342; Prime et al. (1996) J. Chrom. A 720: 263-274); by RAAM sequencing of released oligosaccharides in combination with mass spectrometry (Klausen et al. (1998) Molecular Biotechnology 9: 195-204); or by combining proteolytic degradation, glycopeptide purification by HPLC, exoglycosidase degradations and mass spectrometry (Krogh et al. (1997) Eur. J. Biochem. 244: 334-342). Specific methods for determining the glycosylation profile is described in the examples section hereinafter. Normally, the glycosylated polypeptide of the invention comprises 1-15 oligosaccharide moieties, such as 1-10 or 1-6 oligosachharide moieties. Usually, at least one of these is attached to the peptide addition and further oligosaccharide structures are attached to the peptide addition or the polypeptide Pp.

Polypeptide of the Invention Conjugated to a Second Non-Peptide Moiety

It can be advantageous that the glycosylated polypeptide of the invention further comprises at least one second non-peptide moiety. The term “second non-peptide moeity” is intended to indicate a non-peptide moiety different from an oligosaccharide moiety, e.g., a polymer molecule, a lipophilic compound and an organic derivatizing agent.

For this purpose the polypeptide must comprise at least one attachment group for the second non-peptide moiety. The attachment group can be one present on an amino acid residue, e.g., selected from the group consisting of the N-terminal or C-terminal amino acid residue of the polypeptide of the invention, lysine, cysteine, arginine, glutamine, aspartic acid, glutamic acid, serine, tyrosine, histidine, phenylalanine and tryptophan, or on an oligosaccharide moiety attached to the polypeptide. For instance, the attachment group for the non-peptide moiety is an epsilon-amino group.

It will be understood that an attachment group for the second non-peptide moiety may be provided by the N-terminal peptide addition, within the polypeptide Pp, and/or as a C-terminal peptide addition (having similar properties to those described above for the peptide addition X). In one embodiment, the peptide addition X comprising or contributing to an attachment site further comprises an attachment group for a second non-peptide moeity. For instance, the peptide addition may comprise 1-20, such as 1-10 attachment groups for a second non-peptide moiety. Such attachment groups may be distributed in a similar manner as that described immediately above for glycosylation sites. Also, the peptide addition X can comprise at least two attachment groups for the second non-peptide moiety.

Also, the polypeptide Pp can be a variant of a native polypeptide, which as compared to said native polypeptide, comprises at least one introduced and/or at least one removed attachment group for the second non-peptide moiety. For instance, the polypeptide. Pp comprises at least one introduced attachment group, in particular 1-5 introduced attachment groups, such as 2-5 or 3-5 introduced attachment groups.

The attachment group is preferably located in a position that is exposed at the surface of the folded protein and thus accessible for conjugation to the polymer molecule. For instance, attachment to one or more polymer molecules increases the molecular weight of the polypeptide and can further serve to shield one or more epitopes thereof. The polymer molecule may be any of the molecules mentioned in the section entitled “Conjugation to a polymer molecule,” but is preferably selected from the group consisting of linear or branched polyethylene glycol or polyalkylene oxide. Most preferably, the polymer molecule is mPEG-SPA, mPEG-SCM, mPEG-BTC from Shearwater Polymers, Inc, SC-PEG from Enzon, Inc., tresylated mPEG (U.S. Pat. No. 5,880,255) or oxycarbonyl-oxy-N-dicarboxyimide PEG (U.S. Pat. No. 5,122,614) (and the relevant attachment group is one present on a lysine or N-terminal residue). Alternatively, the polymer molecule is an activated PEG molecule reactive with a cysteine residue, e.g., VS-PEG from Shearwater Polymers.

Especially, when the polypeptide Pp is an industrial enzyme, the second non-peptide moiety may be one which is capable of cross-linking and thereby of being immobilized on a suitable solid support. Such cross-linking polymers are available from Shearwater Polymers, Inc. It will be understood that the peptide addition of the polypeptide according to this embodiment comprises an attachment group for the cross-linking polymer in question. In connection with this embodiment, the polypeptide Pp is preferably an amyloglucosidase, an alpha-amylase, a glucose isomerase, an amidase, or a lipolytic enzyme.

In the following sections “Conjugation to a lipophilic compound,” “Conjugation to a polymer molecule,” and “Conjugation to an organic derivatizing agent” conjugation to specific types of non-peptide moieties is described.

It will be understood that a conjugation step of any method of the invention only finds relevance when a non-polypeptide moiety other than an in vivo attached oligosaccharide moiety is to be conjugated to the polypeptide, since in vivo glycosylation takes place during the expression step when using an appropriate glycosylating host cell as expression host. Accordingly, whenever a conjugation step occurs in the present invention this is intended to be conjugation to a non-polypeptide moiety other than an oligosaccharide moiety attached by in vivo glycosylation during expression in a glycosylating organism. In vitro glycosylation methods are described in the section entitled “glycosylation.”

Conjugation to a Lipophilic Compound

The polypeptide and the lipophilic compound can be conjugated to each other, either directly or by use of a linker. The lipophilic compound can be a natural compound such as a saturated or unsaturated fatty acid, a fatty acid diketone, a terpene, a prostaglandin, a vitamine, a carotenoide or steroide, or a synthetic compound such as a carbon acid, an alcohol, an amine and sulphonic acid with one or more alkyl-, aryl-, alkenyl- or other multiple unsaturated compounds. Furthermore, the lipophilic compound may be any of the lipophilic substituents disclosed in WO 97/31022, the contents of which are incorporated herein by reference. The conjugation between the polypeptide and the lipophilic compound, optionally through a linker can be done according to methods known in the art, e.g., as described by Bodanszky (1976), in Peptide Synthesis, John Wiley, New York and in WO 96/12505 and further as described in WO 97/31022.

Conjugation to a Polymer Molecule

The polymer molecule to be coupled to the polypeptide of the invention can be any suitable polymer molecule, such as a natural or synthetic homo-polymer or heteropolymer, typically with a molecular weight in the range of 300-100,000 Da, such as 300-20,000 Da, more preferably in the range of 500-10,000 Da, even more preferably in the range of 500-5000 Da.

Examples of homo-polymers include a polyol (i.e., poly-OH), a polyamine (i.e., poly-NH₂) and a polycarboxylic acid (i.e., poly-COOH). A hetero-polymer is a polymer that comprises different coupling groups, such as a hydroxyl group and an amine group.

Examples of suitable polymer molecules include polymer molecules selected from the group consisting of polyalkylene oxide (PAO), including polyalkylene glycol (PAG), such as polyethylene glycol (PEG) and polypropylene glycol (PPG), branched PEGs, poly-vinyl alcohol (PVA), poly-carboxylate, poly-(vinylpyrolidone), polyethylene-co-maleic acid anhydride, polystyrene-co-malic acid anhydride, dextran, including carboxymethyl-dextran, or any other biopolymer suitable for the intended purpose, such as for reducing immunogenicity and/or increasing functional in vivo half-life and/or serum half-life, or for providing immobilization properties to the polypeptide (as discussed in the section entitled “Polypeptide of interest.” Another example of a polymer molecule is human albumin or another abundant plasma protein. Generally, polyalkylene glycol-derived polymers are biocompatible, non-toxic, non-antigenic, non-immunogenic, have various water solubility properties, and are easily excreted from living organisms.

PEG is the preferred polymer molecule for reducing immunogenicity, allergenicity and/or increasing half-life, since it has only few reactive groups capable of cross-linking compared, e.g., to polysaccharides such as dextran, and the like. In particular, monofunctional PEG, e.g., methoxypolyethylene glycol (mPEG), is of interest since its coupling chemistry is relatively simple (only one reactive group is available for conjugating with attachment groups on the polypeptide). Consequently, the risk of cross-linking is eliminated, the resulting polypeptide conjugates are more homogeneous and the reaction of the polymer molecules with the polypeptide is easier to control.

To effect covalent attachment of the polymer molecule(s) to the polypeptide, the hydroxyl end groups of the polymer molecule must be provided in activated form, i.e., with reactive functional groups. Suitable activated polymer molecules are commercially available, e.g., from Shearwater Polymers, Inc., Huntsville, Ala., USA. Alternatively, the polymer molecules can be activated by conventional methods known in the art, e.g., as disclosed in WO 90/13540. Specific examples of activated linear or branched polymer molecules for use in the present invention are described in the Shearwater Polymers, Inc. 1997 and 2000 Catalogs (Functionalized Biocompatible Polymers for Research and pharmaceuticals, Polyethylene Glycol and Derivatives, incorporated herein by reference). Specific examples of activated PEG polymers include the following linear PEGs: NHS-PEG (e.g., SPA-PEG, SSPA-PEG, SBA-PEG, SS-PEG, SSA-PEG, SC-PEG, SG-PEG, and SCM-PEG), and NOR-PEG), BTC-PEG, EPOX-PEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. No. 5,932,462 and U.S. Pat. No. 5,643,575, both of which are incorporated herein by reference. Furthermore, the following publications, incorporated herein by reference, disclose useful polymer molecules and/or PEGylation chemistries: U.S. Pat. No. 5,824,778. U.S. Pat. No. 5,476,653, WO 97/32607, EP 229,108, EP 402,378, U.S. Pat. No. 4,902,502, U.S. Pat. No. 5,281,698, U.S. Pat. No. 5,122,614, U.S. Pat. No. 5,219,564. WO 92/116555, WO 94/04193, WO 94/14758, WO 94/17039, WO 94/18241, WO 94/28024, WO 95/00162, WO 95/11924, WO95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. No. 5,473,034, U.S. Pat. No. 5,516,673, EP 605 963, U.S. Pat. No. 5,382,657, EP 510 356, EP 400 472, EP 183 503 and EP 154 316.

The conjugation of the polypeptide and the activated polymer molecules is conducted by use of any conventional method, e.g., as described in the following references (which also describe suitable methods for activation of polymer molecules): R. F. Taylor (1991) Protein immobilisation: Fundamental and applications Marcel Dekker, N.Y.; S. S. Wong (1992) Chemistry of protein Conjugation and Crosslinking CRC Press, Boca Raton; G. T. Hermanson et al. (1993) Immobilized Affinity Ligand Techniques Academic Press, N.Y.). The skilled person will be aware that the activation method and/or conjugation chemistry to be used depends on the attachment group(s) of the polypeptide (examples of which are given further above), as well as the functional groups of the polymer (e.g., being amine, hydroxyl, carboxyl, aldehyde, sulfydryl, succinimidyl, maleimide, vinysulfone or haloacetate). The PEGylation can be directed towards conjugation to all available attachment groups on the polypeptide (i.e., such attachment groups that are exposed at the surface of the polypeptide) or can be directed towards one or more specific attachment groups, e.g., the N-terminal amino group (U.S. Pat. No. 5,985,265). Furthermore, the conjugation can be achieved in one step or in a stepwise manner (e.g., as described in WO 99/55377).

It will be understood that the PEGylation is designed so as to produce the optimal molecule with respect to the number of PEG molecules attached, the size and form of such molecules (e.g., whether they are linear or branched), and where in the polypeptide such molecules are attached. For instance, the molecular weight of the polymer to be used can be chosen on the basis of the desired effect to be achieved. For instance, if the primary purpose of the conjugation is to achieve a polypeptide having a high molecular weight (e.g., to reduce renal clearance) it is usually desirable to conjugate as few high MW polymer molecules as possible to obtain the desired molecular weight. When a high degree of epitope shielding is desirable this can be obtained by use of a sufficiently high number of low molecular weight polymer molecules (e.g., with a molecular weight of about 5,000 Da) to effectively shield all or most epitopes of the polypeptide. For instance, 2-8, such as 3-6 such polymers can be used.

In connection with conjugation to only a single attachment group on the protein (as described in U.S. Pat. No. 5,985,265), it can be advantageous that the polymer molecule, which can be linear or branched, has a high molecular weight, e.g., about 20 kDa.

Normally, the polymer conjugation is performed under conditions aiming at reacting all available polymer attachment groups with polymer molecules. Typically, the molar ratio of activated polymer molecules to polypeptide is up to about 1000-1, in particular 200-1, preferably 100-1, such as 10-1 or 5-1, but also equimolar ratios can be used in order to obtain optimal reaction.

It is also contemplated according to the invention to couple the polymer molecules to the polypeptide through a linker. Suitable linkers are well known to the skilled person. A preferred example is cyanuric chloride (Abuchowski et al. (1977) J. Biol. Chem. 252: 3578-3581; U.S. Pat. No. 4,179,337; Shafer et al. (1986) J. Polym. Sci. Polym. Chem. Ed. 24: 375-378.

Subsequent to the conjugation residual activated polymer molecules are blocked according to methods known in the art, e.g., by addition of primary amine to the reaction mixture, and the resulting inactivated polymer molecules are removed by a suitable method.

In a specific embodiment, the polypeptide of the invention is one that comprises one or more PEG molecules attached to the peptide addition, but not to the polypeptide P. For instance, the PEG molecule is attached to one or more cysteine residues present in the peptide addition X and, if necessary, one or more cysteine residues have been removed from the polypeptide P of interest in order to avoid conjugation thereto.

In another specific embodiment, the polypeptide of the invention comprises at least one PEG molecule attached to a lysine residue of the peptide addition X, in particular a linear or branched PEG molecule with a molecular weight of at least 5 kDa.

Methods of Preparing a Polypeptide of the Invention

The invention further comprises a method of producing the polypeptide of the invention, which method comprises culturing a host cell transformed or transfected with a nucleotide sequence encoding the polypeptide under conditions permitting the expression of the polypeptide, and recovering the polypeptide from the culture.

Apart from recombinant production, polypeptides of the invention may be produced, albeit less efficiently, by chemical synthesis or a combination of chemical synthesis and recombinant DNA technology.

The nucleotide sequence of the invention encoding a polypeptide of the invention may be constructed by isolating or synthesizing a nucleotide sequence encoding the parent polypeptide and fusing a nucleotide sequence encoding the relevant peptide addition in accordance with established technologies. To the extent amino acid modifications are to be made in the parent polypeptide, these are conveniently done by mutagenesis, e.g., using site-directed mutagenesis in accordance with well-known methods, e.g., as described in Nelson and Long (1989) Analytical Biochemistry 180: 147-151, random mutagenesis, or shuffling.

The nucleotide sequence may be prepared by chemical synthesis, e.g., by using an oligonucleotide synthesizer, wherein oligonucleotides are designed based on the amino acid sequence of the desired polypeptide, and preferably selecting those codons that are favored in the host cell in which the recombinant polypeptide will be produced. For example, several small oligonucleotides coding for portions of the desired polypeptide may be synthesized and assembled by polymerase chain reaction (PCR), ligation or ligation chain reaction (LCR). The individual oligonucleotides typically contain 5′ or 3′ overhangs for complementary assembly.

Once assembled (by synthesis, site-directed mutagenesis or another method), the nucleotide sequence encoding the polypeptide may be inserted into a recombinant vector and operably linked to control sequences necessary for expression of thereof in the desired transformed host cell.

It should of course be understood that not all vectors and expression control sequences function equally well to express the nucleotide sequence encoding the polypeptide part of the invention. Neither will all hosts function equally well with the same expression system. However, one of skill in the art can make a selection among these vectors, expression control sequences and hosts without undue experimentation. For example, in selecting a vector, the host must be considered because the vector must replicate in it or be able to integrate into the chromosome. The vector's copy number, the ability to control that copy number, and the expression of any other proteins encoded by the vector, such as antibiotic markers, should also be considered. In selecting an expression control sequence, a variety of factors should also be considered. These include, for example, the relative strength of the sequence, its controllability, and its compatibility with the nucleotide sequence encoding the polypeptide, particularly as regards potential secondary structures. Hosts should be selected by consideration of their compatibility with the chosen vector the toxicity of the product coded for by the nucleotide sequence, their secretion characteristics their ability to fold the polypeptide correctly, their fermentation or culture requirements, and the ease of purification of the products coded for by the nucleotide sequence.

The recombinant vector may be an autonomously replicating vector, i.e., a vector existing as an extrachromosomal entity, the replication of which is independent of chromosomal replication, e.g., a plasmid. Alternatively, the vector is one which, when introduced into a host cell, is integrated into the host cell genome and replicated together with the chromosome(s) into which it has been integrated.

The vector is preferably an expression vector, in which the nucleotide sequence encoding the polypeptide of the invention is operably linked to additional segments required for transcription of the nucleotide sequence. The vector is typically derived from plasmid or viral DNA. A number of suitable expression vectors for expression in the host cells mentioned herein are commercially available or described in the literature. Useful expression vectors for eukaryotic hosts, include, for example, vectors comprising expression control sequences from SV40, bovine papilloma virus, adenovirus and cytomegalovirus. Specific vectors are, e.g., pCDNA3.1 (+)\Hyg (Invitrogen, Carlsbad, Calif., USA) and pCI-neo (Stratagene, La Jolla, Calif., USA). Useful expression vectors for yeast cells include the 2μ plasmid and derivatives thereof, the POT1 vector (U.S. Pat. No. 4,931,373), the pJSO37 vector described in (Okkels, Ann. New York Acad. Sci. 782, 202-207, 1996) and pPICZ A, B or C (Invitrogen, Carlsbad, Calif., USA). Useful vectors for insect cells include pVL941, pBG311 (Cate et al. (1986) “Isolation of the Bovine and Human Genes for Mullerian Inhibiting Substance And Expression of the Human Gene In Animal Cells” Cell 45: 685-98, pBluebac 4.5 and pMelbac (both available from Invitrogen, Carlsbad, Calif., USA).

Other vectors for use in this invention include those that allow the nucleotide sequence encoding the polypeptide of the invention to be amplified in copy number. Such amplifiable vectors are well known in the art. They include, for example, vectors able to be amplified by DHFR amplification (see, e.g., Kaufinan, U.S. Pat. No. 4,470,461, Kaufinan and Sharp (1982) “Construction Of A Modular Dihydrafolate Reductase cDNA Gene: Analysis Of Signals Utilized For Efficient Expression” Mol. Cell. Biol. 2: 1304-19) and glutamine synthetase (“GS”) amplification (see, e.g., U.S. Pat. No. 5,122,464 and EP 338,841).

The recombinant vector may further comprise a DNA sequence enabling the vector to replicate in the host cell in question. An example of such a sequence (when the host cell is a mammalian cell) is the SV40 origin of replication. When the host cell is a yeast cell, suitable sequences enabling the vector to replicate are the yeast plasmid 2μ replication genes REP 1-3 and origin of replication.

The vector may also comprise a selectable marker, e.g., a gene the product of which complements a defect in the host cell, such as the gene coding for dihydrofolate reductase (DHFR) or the Schizosaccharomyces pombe TPI gene (described by P. R. Russell (1985) Gene 40: 125-130), or one which confers resistance to a drug, e.g., ampicillin, kanamycin, tetracyclin, chloramphenicol, neomycin, hygromycin or methotrexate. For filamentous fungi, selectable markers include amdS, pyrG, arcB, niaD, sC.

The term “control sequences” is defined herein to include all components, which are necessary or advantageous for the expression of the polypeptide of the invention. Each control sequence may be native or foreign to the nucleic acid sequence encoding the polypeptide. Such control sequences include, but are not limited to, a leader, polyadenylation sequence, propeptide sequence, promoter, enhancer or upstream activating sequence, signal peptide sequence, and transcription terminator. At a minimum, the control sequences include a promoter operably linked to the nucleotide sequence encoding the polypeptide.

“Operably linked” refers to the covalent joining of two or more nucleotide sequences, by means of enzymatic ligation or otherwise, in a configuration relative to one another such that the normal function of the sequences can be performed. For example, the nucleotide sequence encoding a presequence or secretory leader is operably linked to a nucleotide sequence for a polypeptide if it is expressed as a preprotein that participates in the secretion of the polypeptide: a promoter or enhancer is operably linked to a coding sequence if it affects the transcription of the sequence; a ribosome binding site is operably linked to a coding sequence if it is positioned so as to facilitate translation. Generally, “operably linked” means that the nucleotide sequences being linked are contiguous and, in the case of a secretory leader, contiguous and in reading phase. Linking is accomplished by ligation at convenient restriction sites. If such sites do not exist, then synthetic oligonucleotide adaptors or linkers are used, in conjunction with standard recombinant DNA methods.

A wide variety of expression control sequences may be used in the present invention. Such useful expression control sequences include the expression control sequences associated with structural genes of the foregoing expression vectors as well as any sequence known to control the expression of genes of prokaryotic or eukaryotic cells or their viruses, and various combinations thereof.

Examples of suitable control sequences for directing transcription in mammalian cells include the early and late promoters of SV40 and adenovirus, e.g., the adenovirus 2 major late promoter, the MT-1 (metallothionein gene) promoter, the human cytomegalovirus immediate-early gene promoter (CMV), the human elongation factor 1α (EF-1α) promoter, the Drosophila minimal heat shock protein 70 promoter, the Rous Sarcoma Virus (RSV) promoter, the human ubiquitin C (UbC) promoter, the human growth hormone terminator, SV40 or adenovirus E1b region polyadenylation signals and the Kozak consensus sequence (Kozak (1987) J Mol Biol 196: 947-50).

In order to improve expression in mammalian cells a synthetic intron may be inserted in the 5′ untranslated region of the nucleotide sequence encoding the polypeptide of the invention. An example of a synthetic intron is the synthetic intron from the plasmid pCI-Neo (available from Promega Corporation, WI, USA).

Examples of suitable control sequences for directing transcription in insect cells include the polyhedrin promoter, the P10 promoter, the Autographa californica polyhedrosis virus basic protein promoter, the baculovirus immediate early gene 1 promoter and the baculovirus 39K delayed-early gene promoter, and the SV40 polyadenylation sequence.

Examples of suitable control sequences for use in yeast host cells include the promoters of the yeast α-mating system, the yeast triose phosphate isomerase (TPI) promoter, promoters from yeast glycolytic genes or alcohol dehydogenase genes, the ADH2-4c promoter and the inducible GAL promoter.

Examples of suitable control sequences for use in filamentous fungal host cells include the ADH3 promoter and terminator, a promoter derived from the genes encoding Aspergillus oryzae TAKA amylase triose phosphate isomerase or alkaline protease, an A. niger α-amylase, A. niger or A. nidulas glucoamylase, A. nidulans acetamidase, Rhizomucor miehei aspartic proteinase or lipase, the TPI1 terminator and the ADH3 terminator.

The nucleotide sequence of the invention may or may not also include a nucleotide sequence that encode a signal peptide. The signal peptide is present when the polypeptide is to be secreted from the cells in which it is expressed. Such signal peptide, if present, should be one recognized by the cell chosen for expression of the polypeptide. The signal peptide may be homologous (e.g., be that normally associated with the parent polypeptide in question) or heterologous (i.e., originating from another source than the parent polypeptide) to the polypeptide or may be homologous or heterologous to the host cell, i.e., be a signal peptide normally expressed from the host cell or one which is not normally expressed from the host cell. Accordingly, the signal peptide may be prokaryotic, e.g., derived from a bacterium, or eukaryotic, e.g., derived from a mammalian, or insect, filamentous fungal or yeast cell.

The presence or absence of a signal peptide will, e.g., depend on the expression host cell used for the production of the polypeptide, the protein to be expressed (whether it is an intracellular or extracelluar protein) and whether it is desirable to obtain secretion. For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. For use in insect cells, the signal peptide may conveniently be derived from an insect gene (cf. WO 90/05783), such as the lepidopteran Manduca sexta adipokinetic hormone precursor, (cf. U.S. Pat. No. 5,023,328), the honeybee melittin (Invitrogen, Carlsbad, Calif., USA), ecdysteroid UDP glucosyltransferase (egt) (Murphy et al. (1993) Protein Expression and Purification 4: 349-357, or human pancreatic lipase (hpl) (Methods in Enzymology (1997) 284: 262-272).

Specific examples of signal peptides for use in mammalian cells include that of human glucocerebrosidase apparent from the examples hereinafter or the murine Ig kappa light chain signal peptide (Coloma, M (1992) J. Imm. Methods 152: 89-104). For use in yeast cells suitable signal peptides have been found to be the α-factor signal peptide from S. cereviciae. (cf U.S. Pat. No. 4,870,008), the signal peptide of mouse salivary amylase (cf. O. Hagenbuchle et al. (1981) Nature 289: 643-646), a modified carboxypeptidase signal peptide (cf. L. A. Valls et al. (1987) Cell 48: 887-897), the yeast BAR1 signal peptide (cf WO 87/02670), and the yeast aspartic protease 3 (YAP3) signal peptide (cf. M. Egel-Mitani et al. (1990) Yeast 6: 127-137).

Any suitable host may be used to produce the polypeptide of the invention, including bacteria, fungi (including yeasts), plant, insect mammal, or other appropriate animal cells or cell lines, as well as transgenic animals or plants. When a non-glycosylating organism such as E. coli is used, and the polypeptide is to be a glycosylated polypeptide, the expression in E. coli is preferably followed by suitable in vitro glycosylation.

Examples of bacterial host cells include grampositive bacteria such as strains of Bacillus, e.g., B. brevis or B. subtilis, Pseudomonas or Streptomyces, or gramnegative bacteria, such as strains of E. coli. The introduction of a vector into a bacterial host cell may, for instance, be effected by protoplast transformation (see, e.g., Chang and Cohen (1979) Molecular General Genetics 168: 111-115), using competent cells (see, e.g., Young and Spizizin (1961) Journal of Bacteriology 81: 823-829, or Dubnau and Davidoff-Abelson (1971) Journal of Molecular Biology 56: 209-221), electroporation (see, e.g., Shigekawa and Dower (1988) Biotechniques 6: 742-751), or conjugation (see, e.g., Koehler and Thorne (1987) Journal of Bacteriology 169: 5771-5278).

Examples of suitable filamentous fungal host cells include strains of Aspergillus, e.g., A. oryzae, A. niger, or A. nidulans, Fusarium or Trichoderma. Fungal cells may be transformed by a process involving protoplast formation, transformation of the protoplasts, and regeneration of the cell wall in a manner known per se. Suitable procedures for transformation of Aspergillus host cells are described in EP 238 023 and U.S. Pat. No. 5,679,543. Suitable methods for transforming Fusarium species are described by Malardier et al. (1989) Gene 78: 147-156 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al. (1983) Journal of Bacteriology 153: 163; and Hinnen et al. (1978) Proceedings of the National Academy of Sciences USA 75: 1920.

When the polypeptide of the invention is to be in vivo glycosylated, the host cell is selected from a group of host cells capable of generating the desired glycosylation of the polypeptide. Thus, the host cell may advantageously be selected from a yeast cell, insect cell, or mammalian cell.

Examples of suitable yeast host cells include strains of Saccharomyces, e.g., S. cerevisiae, Schizosaccharomyces, Klyveromyces, Pichia, such as P. pastoris or P. methanolica, Hansenula, such as H. polymorpha or yarrowia. Of particular interest are yeast glycosylation mutant cells, e.g., derived from S. cereviciae, P. pastoris or Hansenula spp. (e.g., the S. cereviciae glycosylation mutants och1, ochi mnm1 or och1 mnm1 alg3 described by Nagasu et al. (1992) Yeast 8: 535-547 and Nakanisho-Shindo et al. (1993) J. Biol. Chem. 268: 26338-26345. Methods for transforming yeast cells with heterologous DNA and producing heterologous polypeptides therefrom are disclosed by Clontech Laboratories, Inc, Palo Alto, Calif., USA (in the product protocol for the Yeastmaker™ Yeast Tranformation System Kit), and by Reeves et al. (1992) FEMS Microbiology Letters 99: 193-198, Manivasakam and Schiestl (1993) Nucleic Acids Research 21: 4414-4415 and Ganeva et al. (1994) FEMS Microbiology Letters 121: 159-164.

Examples of suitable insect host cells include a Lepidoptora cell line, such as Spodoptera frugiperda (Sf9 or Sf21) or Trichoplusia ni cells (High Five) (U.S. Pat. No. 5,077,214). Transformation of insect cells and production of heterologous polypeptides therein may be performed as described by Invitrogen, Carlsbad, Calif., USA.

Examples of suitable mammalian host cells include Chinese hamster ovary (CHO) cell lines, (e.g., CHO-K1; ATCC CCL-61), Green Monkey cell lines (COS) (e.g., COS 1 (ATCC CRL-1650), COS 7 (ATCC CRL-1651)); mouse cells (e.g., NS/O), Baby Hamster Kidney (BHK) cell lines (e.g., ATCC CRL-1632 or ATCC CCL-10), and human cells (e.g., HEK 293 (ATCC CRL-1573)), as well as plant cells in tissue culture. Additional suitable cell lines are known in the art and available from public depositories such as the American Type Culture Collection, Rockville, Md. Of interest for the present purpose are a mammalian glycosylation mutant cell line, such as CHO-LEC1, CHOL-LEC2 or CHO-LEC18 (CHO-LEC1: Stanley et al. (1975) Proc. Natl. Acad. USA 72: 3323-3327 and Grossmann et al.(1995) J. Biol. Chem. 270: 29378-29385, CHO-LEC18: Raju et al. (1995) J. Biol. Chem. 270: 30294-30302).

Methods for introducing exogeneous DNA into mammalian host cells include calcium phosphate-mediated transfection, electroporation, DEAE-dextran mediated transfection, liposome-mediated transfection, viral vectors and the transfection method described by Life Technologies Ltd, Paisley, UK using Lipofectamin 2000. These methods are well known in the art and e.g., described by Ausbel et al. (eds.) (1996) Current Protocols in Molecular Biology John Wiley & Sons, New York, USA. The cultivation of mammalian cells are conducted according to established methods, e.g. as disclosed in Jenkins, Ed. (1999) Animal Cell Biotechnology, Methods and Protocols Human Press Inc. Totowa, N.J., USA; and Harrison and Rae (1997) General Techniques of Cell Culture Cambridge University Press.

In the production methods of the present invention, cells are cultivated in a nutrient medium suitable for production of the polypeptide using methods known in the art. For example, cells are cultivated by shake flask cultivation, small-scale or large-scale fermentation (including continuous, batch, fed-batch, or solid state fermentations) in laboratory or industrial fermenters performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The resulting polypeptide may be recovered by methods known in the art. For example, the polypeptide may be recovered from the nutrient medium by conventional procedures including, but not limited to, centrifugation, filtration, extraction, spray drying, evaporation, or precipitation.

The polypeptides may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., J-C Janson and Lars Ryden, editors (1989) Protein Purification VCH Publishers, New York).

Other Methods of the Invention

In accordance with a specific aspect a nucleotide sequence encoding the polypeptide of the invention is prepared by a method comprising:

a) subjecting a nucleotide sequence encoding the polypeptide Pp to elongation mutagenesis;

b) expressing the mutated nucleotide sequence obtained in step a) in a suitable host cell;

c) optionally conjugating polypeptides expressed in step b) to a second non-peptide moiety;

d) selecting polypeptides of step b) or c) which comprises at least one oligosaccharide moiety and optionally second non-peptide moiety attached to the peptide addition part of the polypeptide; and,

e) isolating a nucleotide sequence encoding the polypeptide selected in step d).

In the present context the term “elongation mutagenesis” is intended to indicate any manner in which the nucleotide sequence encoding the parent polypeptide Pp can be extended to further encode the peptide addition. For instance, a nucleotide sequence encoding a peptide addition of a suitable length may be synthesized and fused to a nucleotide sequence encoding the polypeptide Pp. The resulting fused nucleotide sequence may then be subjected to further modification by any suitable method, e.g., one which involves gene shuffling, other recombination between nucleotide sequences, random mutagenesis, random elongation n. tagenesis or any combination of these methods. Such methods are further described in the Methods section herein.

The expression and optional conjugation steps are conducted as described in further detail elsewhere in the present application, and the selection step d) using any suitable method available in the art.

In one embodiment, the above method further comprises screening polypeptides resulting from step b) or c) for at least one improved property, in particular any of those improved properties listed herein, prior to the selection step, and wherein the selection step d) further comprises selecting polypeptides having such improved property.

Furthermore, in the above method the elongation mutagenesis can be conducted so as to enrich for codons encoding a glycosylation site and/or an amino acid residue comprising an attachment group for a second non-peptide moiety, in particular an in vivo glycosylation site.

Still further, the above method can comprise subjecting the part of the nucleotide sequence encoding the polypeptide Pp of interest to mutagenesis to remove and/or introduce glycosylation site(s) and/or amino acid residue(s) comprising an attachment group for the second non-peptide moiety. The nucleotide sequence may be subjected to any type of mutagenesis, e.g., any of those described herein. The mutagenesis of the nucleotide sequence encoding the polypeptide Pp of interest can be conducted prior to assembling the sequence with that encoding the peptide addition, concomitantly with or after any mutagenesis of the peptide addition part of the assembled nucleotide sequence.

In a further aspect, the invention relates to a method of producing a glycosylated polypeptide encoded by a nucleotide sequence of the invention prepared by the above method, wherein the nucleotide sequence encoding the polypeptide selected in step c) is expressed in a glycosylating host cell and the resulting glycosylated expressed polypeptide is recovered.

In a still further aspect the invention relates to a method of improving one or more selected properties of a polypeptide Pp of interest, which method comprises:

a) preparing a nucleotide sequence encoding a polypeptide comprising or consisting essentially of the primary structure NH₂-X-Pp-COOH, wherein X is a peptide addition comprising or contributing to a glycosylation site and/or an attachment group for a second non-peptide moiety that is capable of conferring the selected improved property/ie to the polypeptide Pp;

b) expressing the nucleotide sequence of a) in an suitable host cell;

c) optionally conjugating the expressed polypeptide of b) to a second non-peptide moiety; and,

d) recovering the polypeptide resulting from step b) or c).

For instance, the polypeptide is any of those described herein. For instance the nucleotide sequence of step a) is prepared by subjecting a nucleotide sequence encoding the polypeptide Pp to elongation mutagenesis, e.g., to enrich for codons encoding an amino acid residue comprising or contributing to a glycosylation site and/or an attachment group for a second non-peptide moiety, in particular an in vivo glycosylation site. Also, in the preparation of the nucleotide sequence of a), the part of the nucleotide sequence encoding the polypeptide Pp can be subjected to mutagenesis to remove and/or introduce glycosylation site(s) and/or attachment group(s) for a second non-peptide moiety.

The method according to this aspect can further comprise a screening step (after step c)), wherein the polypeptide resulting from step b) or c) is screened for one or more improved properties, in particular any of those improved properties which are described hereinabove.

Usually, when a polypeptide has been selected in a screening step of a method of the invention the nucleotide sequence encoding the polypeptide is isolated and used for expression of larger amounts of the polypeptide. The amino acid sequence of the resulting polypeptide is determined and the polypeptide may be subjected to conjugation in a larger scale. Subsequently, the polypeptide is assayed with respect to the property to be improved.

Uses of a Polypeptide of the Invention

It will be understood that polypeptides of the invention can be used for a variety of purposes, depending on the type and nature of polypeptide. For instance, it is contemplated that a polypeptide of the invention prepared from a therapeutic polypeptide is useful for the same therapeutic purposes as the parent polypeptide, i.e., for the treatment of a particular disease. Accordingly, the polypeptide of the invention may be formulated into a pharmaceutical composition. Also, when the polypeptide of the invention is an in vivo glycosylated polypeptide which does not comprise any other type of non-peptide moiety, a nucleotide sequence encoding the polypeptide can be used in gene therapy in accordance with established principles. When the polypeptide Pp is an antigen the polypeptide of the invention may be provided in the form of a vaccine.

### Methods

Nucleotide Sequence Modification Methods

For example, a peptide addition may be constructed from two or more nucleotide sequences encoding a polypeptide of interest with a peptide addition, the sequences being sufficiently homologous to allow recombination between the sequences, in particular in the part thereof encoding the peptide addition. The combination of nucleotide sequences or sequence parts is conveniently conducted by methods known in the art, for instance methods which involve homologous cross-over such as disclosed in U.S. Pat. No. 5,093,257, or methods which involve gene shuffling, i.e., recombination between two or more homologous nucleotide sequences resulting in new nucleotide sequences having a number of nucleotide alterations when compared to the starting nucleotide sequences. In order for homology based nucleic acid shuffling to take place the relevant parts of the nucleotide sequences are preferably at least 50% identical, such as at least 60% identical, more preferably at least 70% identical, such as at least 80% identical. The recombination can be performed in vitro or in vivo. Examples of suitable in vitro gene shuffling methods are disclosed by Stemmer et al (1994) Proc. Natl. Acad. Sci. USA 91: 10747-10751; Stemmer (1994) Nature 370: 389-391; Smith (1994) Nature 370: 324-325; Zhao et al. Nat. Biotechnol. (1998) 16(3): 258-61; Zhao H, and Arnold, F B Nucleic Acids Research (1997) 25: 1307-1308; Shao et al. (1998) Nucleic Acids Research 26(2): 681-83; and WO 95/17413. Example of a suitable in vivo shuffling method is disclosed in WO 97/07205.

Furthermore, a peptide addition can be constructed by preparing a randomly mutagenized library, conveniently prepared by subjecting a nucleotide sequence encoding the polypeptide of the invention or the peptide addition to random mutagenesis to create a large number of mutated nucleotide sequences. While the random mutagenesis can be entirely random, both with respect to where in the nucleotide sequence the mutagenesis occurs and with respect to the nature of mutagenesis, it is preferably conducted so as to randomly mutate only the part of the sequence that encode the peptide addition. Also, the random mutagenesis can be directed towards introducing certain types of amino acid residues, in particular amino acid residues containing an attachment group, at random into the polypeptide molecule or at random into peptide addition part thereof. Besides substitutions, random mutagenesis can also cover random introduction of insertions or deletions. Preferably, the insertions are made in reading frame, e.g., by performing multiple introduction of three nucleotides as described by Hallet et al. (1997) Nucleic Acids Res 25(9): 1866-7 and Sondek and Shrotle (1992) Proc Nat. Acad. Sci USA 89(8): 3581-5.

The random mutagenesis (either of the whole nucleotide sequence or more preferably the part thereof encoding the peptide addition) can be performed by any suitable method. For example, the random mutagenesis is performed using a suitable physical or chemical mutagenizing agent, a suitable oligonucleotide, PCR generated mutagenesis or any combination of these mutagenizing agents and/or other methods according to state of the art technology, e.g., as disclosed in WO 97/07202.

Error prone PCR generated mutagenesis, e.g., as described by J. O. Deshler (1992) GATA 9(4): 103-106 and Leung et al. (1989) Technique 1: 11-15, is particularly useful for mutagenesis of longer peptide stretches (corresponding to nucleotide sequences containing more than 100 bp) or entire genes, and are preferably performed under conditions that increase the misincorporation of nucleotides.

Random mutagenesis based on doped or spiked oligonucleotides or by specific sequence oligonucleotides, is of particular use for mutagenesis of the part of the nucleotide sequence encoding the peptide addition.

Random mutagenesis of the part of the nucleotide sequence encoding the peptide addition can be performed using PCR generated mutagenesis, in which one or more suitable oligonucleotide primers flanking the area to be mutagenized are used. In addition, doping or spiking with oligonucleotides can be used to introduce mutations so as to remove or introduce attachment groups for the relevant non-peptide moiety. State of the art knowledge and computer programs (e.g., as described by Siderovski D P and Mak T W (1993) Comput. Biol. Med. 23: 463-474 and Jensen et al. (1998) Nucleic Acids Research 26, No. 3) can be used for calculating the most optimal nucleotide mixture for a given amino acid preference. The oligonucleotides can be incorporated into the nucleotide sequence encoding the peptide addition by any published technique using e.g., PCR, LCR or any DNA polymerase or ligase.

According to a convenient PCR method the nucleotide sequence encoding the polypeptide of the invention and in particular the peptide addition thereof is used as a template and, e.g., doped or specific oligonucleotides are used as primers. In addition, cloning primers localized outside the targetted region can be used. The resulting PCR product can either directly be cloned into an appropriate expression vector or gel purified and amplified in a second PCR reaction using the cloning primers and cloned into an appropriate expression vector.

In addition to the random mutagenesis methods described herein, it is occasionally useful to employ site specific mutagenesis techniques to modify one or more selected amino acids in the peptide addition, in particular to optimise the peptide addition with respect to the number of attachment groups.

Furthermore, random elongation mutagenesis as described by Matsuura et al, op cit can be used to construct a nucleotide sequence encoding a polypeptide having a C-terminal peptide addition. Construction of a nucleotide sequence encoding the polypeptide of the invention having an N-terminal peptide addition can be constructed in an analogous way.

Also, the methods disclosed in WO 97/04079, the contents of which are incorporated herein by reference, can be used for constructing a nucleotide sequence encoding the polypeptide of the invention.

The nucleotide sequence(s) or nucleotide sequence region(s) to be mutagenized is typically present on a suitable vector such as a plasmid or a bacteriophage, which as such is incubated with or otherwise exposed to the mutagenizing agent. The nucleotide sequence(s) to be mutagenized can also be present in a host cell either by being integrated into the genome of said cell or by being present on a vector harboured in the cell. Alternatively, the nucleotide sequence to be mutagenized is in isolated form. The nucleotide sequence is preferably a DNA sequence such as a cDNA, genomic DNA or synthetic DNA sequence.

Subsequent to the incubation with or exposure to the mutagenizing agent, the mutated nucleotide sequence, normally in amplified form, is expressed by culturing a suitable host cell carrying the nucleotide sequence under conditions allowing expression to take place. The host cell used for this purpose is one, which has been transformed with the mutated nucleotide sequence(s), optionally present on a vector, or one which carried the nucleotide sequence during the mutagenesis, or any kind of gene library.

Design of Peptide Addition

One example of a useful guide for designing an N-terminal peptide addition containing N-glycosylation sites is characterized by the following formula:
X₁(NX₂[T/S])X₃(NX₂[T/S])ₙX₄-Pp
wherein each of X₁, X₃ and X₄ independently is absent or 1, 2, 3 or 4 amino acid residues of any type, X₂ a single amino acid residue of any type except for proline, n any integer between 0 and 6, [T/S] a threonine or serine residue, preferably a threonine residue, and N and Pp has the meaning defined elsewhere herein. It has been found that sometimes the nature of the amino acid residue occupying position −1 to −4 relative to the N-residue of an N-glycosylation site may be important for the degree to which said N-glycosylation site is used. Accordingly, X₁, X₃, and X₄ may be chosen so as to obtain an increased utilization of the relevant site (as determined by a trial and error type of experiment). In a first step about 10 different muteins are made that has the above formula. For instance, the about 10 muteins are designed on the basis that each of X₁, X₃ and X₄ independently is 1 or 2 alanine residues or is absent, Z any integer between 0 and 5, [T/S] threonine, and Alanine. Based on, e.g., in vitro bioactivity and half-life results obtained with these muteins (or any other relevant property), optimal number(s) of amino acids and glycosylation(s) can be determined and new muteins can be constructed based on this information. The process is repeated until an optimal glycosylated polypeptide is obtained.

Alternatively, random mutagenesis may be used for creating N-terminally extended polypeptides. For instance, a random mutagenized library is made on the basis of the above formula. Doped oligonucleotides are synthesized coding for one amino acid residue in position B (the amino acid residue being different from proline), each of X₁, X₃, and X₄ independently is 0, 1 or 2 amino acid residues of any type, n is 2 and T is threonine and used for constructing the random mutagenized library.

One example of a useful guide for designing an N-terminal peptide addition containing a PEGylation attachment group is characterized by the following formula using a lysine residue as an example of a PEGylation site. It will be understood that peptide additions with other attachment groups can be designed in an analogous way.
Y¹(K)Y²(K)ₙY³-Pp,
wherein each of Y¹, Y² and Y³ independently is 0, 1, 2, 3 or 4 amino acid residues of any type except lysine, n an integer between 0 and 6, K lysine, and Pp is as defined elsewhere herein.

In a first step about 10 different muteins are made that has the above formula. For instance, the about 10 muteins are designed on the basis that each of Y¹, Y² and Y³ independently is 1 or 2 alanine residues or is absent, n any integer between 0 and 5. The muteins are then PEGylated with 10 kDa PEG (e.g., using mPEG-SPA). Based on e.g., in vitro bioactivity and half-life results obtained with these muteins (or any other relevant property), optimal number(s) of amino acids and PEGylation sites can be determined and new muteins can be constructed based on this information. The process is repeated until an optimal PEGylated polypeptide is obtained.

Alternatively, random mutagenesis may be performed by making a random mutagenized library based on the above formula. Doped oligonucleotides are synthesized coding for one amino acid residue in position Y¹, Y²′ and/or Y³ independently is 0, 1 or 2 amino acid residues of any type, and n is 2 and used for constructing the random mutagenized library.

Glucocerebrosidase (GCB) Activity Assay Using PNP-glucopyranoside Substrate

The enzymatic activity of recombinant GCB is measured using p-nitrophenyl-β-D-glucopyranoside (PNP-Glu) as a substrate. Hydrolysis of the PNP-Glu substrate generates p-nitrophenyl, which can be quantified by measuring absorption at 405 nm using a spectrophotometer, as previously described (Friedmann et al. (1999) Blood 93: 2807-2816). The assay is carried out under conditions which partially inhibit non-GCB glucosidase activities, such conditions being achieved by using a phosphate/citrate buffer pH 5.5, 0.25% Triton X-100 and 0.25% taurocholate.

The assay is run in a final volume of 200 μl, containing GCB Activity Assay Buffer and 4 mM PNP-Glu. The enzymatic hydrolysis is initiated by adding GCB and the reaction is allowed to proceed for 1 hour at 37° C. before being stopped by adding 50 μl 1 M NaOH and measuring absorption at 405 nm. A reference standard curve of p-nitrophenyl, assayed in parallel, is used to quantify concentrations of GCB in samples to be tested.

In Vitro Uptake and Stability of GCB Polypeptide in Macrophages

The murine monocyte/macrophage cells line, J774E (Mukhopadhyay and Stahl (1995) Arch Biochem Biophys 324(1): 78-84 and Diment et al. (1987) J Leukoc Biol 42(5): 485-90) is used to study the uptake and stability of GCB polypeptides. Cells are grown in alpha-MEM (supplemented with 10% fetal calf serum, 1×Pen/Strep, and 60 μM 6-thioguanine), seeded (200,000 cells pr. well) in the above-mentioned media containing 10 μM conditol B epoxide, CBE (an irreversible GCB inhibitor) and incubated for 24 hr at 37° C.

Before starting the uptake assay, cells are washed in 0.5 ml HBSS (Hanks balanced salt solution). The uptake is done in a 200 μl volume, containing the appropriate concentration of GCB polypeptide (a dosis response curve is made with GCB concentrations in the range of 25-400 mU/ml). As a control, yeast mannan (final concentration 1.4 mg/ml) is added to inhibit the uptake through the macrophage mannose receptor. The cells are incubated for 1 hr at 37° C. and washed three times with 0.5 ml cold HBSS.

To measure the amount of GCB taken up by the J774E cells, cells are lyzed in 200 μl GCB Activity Assay Buffer with 4 mM PMP-Glu and incubated for 1 hr at 37° C. Then, the hydrolysis is stopped by addition of 50 μl 1M NaOH and OD405 is measured. The data are analysed by non-linear regression using GraphPad Prizm 2.0 (GraphPad Software, San Diego, Calif.)

To study the stability of GCB polypeptides in J774E cells, CBE treated cells are incubated with 400 mU/ml GCB for 1 hr at 37° C. Then, cells are washed 3 times in HBSS to remove extracellular GCB and incubated in HBSS. A time-course study is done by lyzing the cells after 30 min, 1 hr, 2 hr, 3 hr, 4 hr, and 5 hr in 200 PI GCB Activity Assay Buffer with 4 mM PNP-Glu and incubating the samples for 1 hr at 37° C. before stopping the hydrolysis with 50 μl M NaOH and measuring OD405. The data are analysed by non-linear regression using GraphPad Prizm 2.0 (GraphPad Software, San Diego, Calif.).

Site-Directed Mutagenesis

Constructions of site-directed mutations were performed using PCR with oligonucleotides containing the desired amino acid exchanges or additions (e.g., to introduce glycosylation sites). The resulting PCR fragment was cloned into the GCB expression vector using approparite restriction enzymes and subsequently DNA sequenced in order to confirm that the construct contained the desired exchanges.

Materials

GCB Activity Assay Buffer:

120 mM phosphate/citrate buffer, pH=5.5, 1 mM EDTA, pH=8.0, 0.25% Triton X-100, 0.25% taurocholate, 4 mM β-mercaptoethanol.

pGC-12 vector

pVL1392 (Pharmingen, USA) with GCB wt cDNA sequence (SEQ ID NO 2) inserted between EcoRV and XbaI.

Table 1

Sequence of primers used for cloning the wt GCB coding region and inserting signal peptides into the pGCBmat plasmid as described in Example 1.

SO49 (WT-sp-Bg1II):
5′-CGCAGATCTGATGGCTGGCAGCCTCACAGGATTGC-3′
SO50 (WT-stop-EcoRI):
5′-CCGGAATTCCCATCACTGGCGACGCCACAGGTAGGTG-3′
SO51 (WT-mature-SacI):
5′-ACGCGAGCTCGCCCCTGCATCCCTAAAAGCTTCGG-3′
SO52 (SPegt-NheI/SacI-as):
5′-GCGTTGACGGCAGTCAGAGTTGACAGAAGGGCCAGCCAGCAAAGGAT
AGTCATG-3′
SO53 (SPegt-NheI/SacI-s):
5′-CTAGCATGACTATCCTTTGCTGGCTGGCCCTTCTGTCAACTCTGACT
GCCGTCAACGCAGCT-3′
SO54 (SPegt-NheI/SacI-as):
5′-CCTGCTACTGCTCCCAGCAGCAGTGAAAGAGTCCAAAGTGGCAGCAT
G-3′
SO55 (SPegt-NheI/SacI-s):
5′-CTAGCATGCTGCCACTTTGGACTCTTTCACTGCTGCTGGGAGCAGTA
GCAGGAGCT-3′

Cerezyme was kindly provided by Dr. E. Beutler. Scripps Institute, CA, USA.

J774E was kindly provided by G. Grabowski. Cincinnati, Ohio, US

### EXAMPLE 1

### Production of wt GCB

Cloning and Expression in Insect Cells

A human fibroblast cDNA library was obtained from Clontech (Human fibroblast skin cDNA cloned in lambda-gt11, cat# HL1052b). Lambda DNA was prepared from the library by standard methods and used as a template in a PCR reaction with either SO49 and SO50 as primer (amplifies the GCB coding region with the human signal peptide from the second ATG) or SO50 and SO51 as primer (amplifies the mature part of the GCB coding region) (see Table 1 in the Materials section).

The PCR products were reamplified with the same primers and agarose gel purified. Subsequently the SO49/50 PCR product was digested with BglII and EcoRI and cloned into the pBlueBac 4.5 vector (InVitrogenInvitrogen, Carlsbad, Calif., USA, Carlsbad, Calif., USA) digested with BamHI and EcoRI. Sequencing confirmed that the insert is identical to the wtGCB sequence as given in SEQ ID NO 2. The resulting plasmid was used for infection of insect cells with the GCB being partly secreted from the cells due to the human signal sequence as described in Martin et al., DNA 7, pp. 99-106, 1988. The SO50/51 PCR product was digested with SacI and EcoRI and cloned into the pBlueBac 4.5 vector (InVitrogenInvitrogen, Carlsbad, Calif., USA) digested with the same enzymes resulting in the pGCBmat plasmid. Two different signal sequences were inserted upstream of the mature GCB codons in order to increase the secreted amount of enzyme. The baculovirus ecdysteroid UDP glucosyltransferase (egt) signal sequence (Murphy et al., Protein Expression and Purification 4, 349-357, 1993) was inserted by annealling SO52 and SO53 (Table 1) and the human pancreatic lipase signal sequence (Lowe et al., J. Biol. Chem. 264, 20042, 1989) was inserted by annealling SO54 and SO55 (Table 1) and cloning them into the NheI and SacI digested pGCBmat plasmid. Infection of Spodoptera frugiperda (Sf9) cells of the resulting plasmid was done according to the protocols from InVitrogenInvitrogen, Carlsbad, Calif., USA.

Purification of GCB Polypeptides Produced in Insect Cells

Polypeptides with GCB activity were purified as described in U.S. Pat. No. 5,236,838, with some modifications. Cells were removed from the culture medium by centrifugation (10 min at 4000 rpm in a Sorvall RC5C centrifuge) and the supernatant microfiltrated using a 0.22 μm filter prior to purification. DTT was added to 1 mM and the culture supernatant was ultrafiltrated to approximately 1/10 of the starting volume using a Vivaflow 200 system (Vivascience). The concentrated media was centrifuged to remove possible aggregates before application on a Toyopearl Butyl650C resin (TosoHaas) previously equilibrated in 50 mM sodium citrate, 20% (v/v) ethylene glycol, 1 mM DTT, pH 5.0. This chromatographic step was performed at room temperature. The resin was washed with at least 3 column volumes of 50 mM sodium citrate, 20% (v/v) ethylene glycol, 1 mM DTT, pH 5.0 (until the absorbance at 280 nm reaches baseline level) and GCB was eluted with a linear gradient from 0% to 100% 50 mM sodium citrate, 80% (v/v) ethylene glycol, 1 mM DTT, pH 5.0. Fractions were collected and assayed for GCB activity using the GCB Activity Assay. Usually, wt GCB starts to elute at approx. 70% (v/v) ethylene glycol.

The subsequent purification was done by either of the following two methods. #2 method results in GCB of a higher purity.

Method #1

GCB enriched fractions from the first process step were pooled and diluted approx. 4 times with a buffer containing 50 mM sodium citrate, 5 mM DTT, pH 5.0 to reduce the ethylene glycol content to 20% (or lower). In the second HIC purification step the diluted and partially purified GCB was applied on a Toyopearl phenyl resin (TosoHaas) equilibrated in 50 mM sodium citrate, 1 mM DTT, pH 5.0 (Buffer A) before use. After application, the resin was washed with at least 3 column volumes of 50 mM sodium citrate, pH 5 (until the absorbance at 280 nm reaches baseline level) and GCB was then eluted with a linear ethanol gradient from 0% to 100% buffer B (50 mM sodium citrate. 50% (v/v) ethanol, 1 mM DTT, pH 5.0). Highly purified fractions of GCB (wildtype ≧95% pure), identified using the GCB Activity Assay, start to elute at approx. 40% ethanol. The purified GCB bulk product was dialyzed against 50 mM sodium citrate, 0.2 M mannitol, 0.09% tween80, pH 6.1 to retain the GCB activity upon subsequent storage at 4-8° C. or at −80° C.

Method #2

GCB enriched fractions eluted from the Toyopearl butyl 650C resin were pooled and applied at 4° C. on a SP sepharose resin (Amersham Pharmacia Biotech) previously equilibrated in 25 mM sodium citrate. 1 mM DTT, 10% ethylene glycol, pH 5.0. After application, the resin was washed with 1 mM sodium citrate, 1 nM DTT, 10% ethylene glycol, pH 5.0 (until absorption at 280 nm reached baseline level) and GCB was then eluted with a linear gradient from 0 to 100% 0.25 M sodium citrate, 1 mM DTT, 10% ethylene glycol, pH 5.0. GCB begins to elute around 0.15 M sodium citrate. Fractions containing GCB were pooled and applied at room temperature onto a Phenyl sepharose High Performance (Pharmacia Biotech) previously equilibrated in 25 mM sodium citrate 1 mM DTT, pH 5.0. After application, the resin was washed with 25 mM sodium citrate 1 mM DTT, pH 5.0 until absorption at 280 nm reached baseline level, and GCB was then eluted with a linear ethanol gradient from 0 to 100% 25 mM sodium citrate 1 mM DTT 50% ethanol pH 5.0. GCB typically elutes around 35% ethanol.

The purified GCB bulk product was dialyzed against either 50 mM sodium citrate, 1 mM DTT, pH 5.0 or 50 mM sodium citrate, 0.2 M mannitol, 1 mM DTT, pH 6.1 to retain the GCB activity upon subsequent storage. The purified GCB was concentrated and sterilfiltrered before storage at 4-8° C. or at −80° C. Typically, GCB purified by this method is >95% pure.

### EXAMPLE 2

### Preparation of GCB with N-Terminal Peptide Additions Using a Site-Directed or Randon Mutagenesis Approach

Nucleotide sequences encoding the following N-terminal peptide additions were added to the nucleotide sequence shown in SEQ ID NO 2 encoding wtGCB: (A-4)+(N-3)+(I-2)+(T-1) (representing an extension to the N-terminal of the amino acid sequence shown in SEQ ID NO 1 with the amino acid residues ANIT), and (A-7)+(S-6)+(P-5)+(I-4)+(N-3)+(A-2)+(T-1) (ASPINAT).

A nucleotide sequence encoding the N-terminal peptide addition (A-4)+(N-3)+(I-2)+(T-1) was prepared by PCR using the following conditions:

PCR 1:

Template: 10 ng pBlueBac5 with wt GCB cDNA sequence

primer S060:
5′-CAGCTGGCCATGGGTACCCGG-3′
and
primer S085:
5′-TGGGCATCAGGTGCCAACATTACAGCCCGCCCCTGCATCCCTAAAA
GC-3′

BIO-X-ACTT™ DNA polymerase (Bioline, London. U.K.)
1×OptiBuffer™ (Bioline London, U.K.)
30 cycles of 96° C. 30s, 55° C. 30s, 72° C. 1 min
PCR 2:

Template: 10 ng pBlueBac5 with wt GCB,

Baculo virus forward primer:
5′-TTTACTGTTTTCGTAACAGTTTTG-3′
and
PrimerSO86:
5′-GCAGGGGCGGGCTGTAATGTTGGCACCTGATGCCCACGACACTGCC
TG-3′

BIO-X-ACT™ DNA polymerase (Bioline, London, U.K.)
1×OptiBuffer™ (Bioline, London, U.K.)
30 cycles of 96° C. 30s, 55° C. 30s, 72° C. 1 min
PCR 3:
3 μl of agarose gel purified PCR1 and PCR2 products (app. 10 ng)
Baculo virus forward primer: 5′-TTTACTGTTTTCGTAACAGTTTTG-3′ and primer SO60.
BIO-X-ACT™ DNA polymerase (Bioline, London, U.K.)
1×OptiBuffer™ (Bioline, London, U.K.)
30 cycles of 96° C. 30s, 55° C. 30 s, 72° C. 1 min

PCR 3 was agarose gel purified and digested with NheI and NcoI and cloned into pBluebac4.5+wtGCB digested with NheI and NcoI.

After confirmation of the correct mutations by DNA sequencing the plasmid was transfected into insect cells using the Bac-N-Blue™ transfection kit from Invitrogen, Carlsbad, Calif., USA. Expression of the muteins was tested by western blotting and by activity measurement of the muteins using the GCB Activity Assay.

Enzymatic activity of wtGCB (SEQ ID NO 1) expressed in the expression vector pVL1392 in insect cells (Sf9) using an analogous method to that described in Example 1 gave 13 units/L, while the N-terminal peptide addition ASPINAT gave 28.5 units/L.

Construction of Libraries of GCB with N-Terminal Peptide Addition

Using random mutagenesis two different libraries were constructed on the basis of GCB polypeptides with an N-terminal extension—library A with an N-terminal extension encoding the following amino acid sequence AXNXTXNXTXNXT, and library B with an N-terminal extension encoding ANXTNXTNXT.

Primers for library A were designed:

S0167:
5′-GTGTCGTGGGCATCAGGTGCCNN(G/C)AA(C/T)(T/A/G)N
(G/C)AC(A/T/C)(T/A/G)N(G/C)AA(C/T)(T/A/G)N(G/C)
AC(A/T/C)(T/A/G)N(G/C)AA(C/T)(T/A/G)N(G/C)AC
(A/T/C)GCCCGCCCCTGCATCCCTAAAAGC
S0168:
5′-GGCACCTGATGCCCACGACACTGCCTG

Primers for library B were designed using trinucleotides in the random positions.

X is a mixture of trinucleotide codons for all natural amino acid residues, except proline. The trinucleotide codons used were the same as described by Kayushin et al., Nucleic Acids Research, 24, 3748-3755, 1996.

SO165:
5′-CGTGGGCATCAGGTGCCAAC(X)AC(A/T/C)AA(C/T)(X)AC
(A/T/C)AA(C/T)(X)AC(A/T/C)GCCCGCCCCTGCATCCCTAAA
AGC
SO166:
5′- GTTGGCACCTGATGCCCACGACACTGCCTG
For both libraries:
SO60 and pBR10:
5′- TTT ACT GTT TTC GTA ACA GTT TTG

In all PCR reactions BIO-X-ACT™ DNA polymerase (Bioline, London, U.K.) and 1*Optibuffer™ (Bioline, London, U.K.) were used. The PCR conditions were 30 cycles of 94° C. 30s, 55° C. 1 min, and 72° C. 1 min.

Templates and primers used for preparing a nucleotide sequence encoding the N-terminal extension by the above PCR were as follows:

PCR 1A:

Template: pGC12

Primers: SO60+SO167

PCR 1B:

Template: pGC12

Primers: SO₆₀+SO165

PCR2A:

Template: pGC12

Primers: SO168+pBR10

PCR2B:

Template: pGC12

Primers: SO166+pBR10

PCR3A:

Template: 1 μl of agarose gel purified PCR 1A and 2A products

Primers: SO60+pBR10

PCR 3B:

Template: 1 μl of agarose gel purified PCR 1B and 2B products

Primers: SO₆₀+pBR10

PCR 3A and 3B were agarose gel purified and digested with NheI and NcoI and ligated into pGC-12 digested with NheI and NcoI. The ligation mixture is transformed into competent E. coli. The diversity of the library was examined by DNA sequencing of different E. coli clones and gave rise to the following amino acid sequences:

```
Library A:
  1: AFNXTLNKTWN(F/L)T
  2: TMNNTWNWTWNWT
  3: -EXT wt
  4: ALNSTGNLTVDGT
  5: ASNSTFNLTENLT
  6: TRNVTINCTUNST
  7: -EXT wt
  8: ALNWTYNGTKNVT
  9: AANWTVNFTGNFT
10: -EXT wt
11: AXNXTVNSTUNVT
12: ANNFTFNGTLNLT
13: AGNWTANVTVNVT
14: AGNSTSNVTGNWT
15: AVNSTMNIHAIPP (1 deletion - nonsens)
16: AGNGTVNGTINGT
17: AVNSTGNXTGNWT
18: AGNGTUNGTSNLT
19: -EXT wt
20: AMNSTKNSTLNIT
21: AFNYTSKNST
22: -EXT wt
23: AVNATMNWTANGT
24: ASNSTNNGTLNAT
25: ARNKTKNFTINLT
26: APNITUNDTVNMT
27: AQNKTFNFTMNCT
28: ALNVTWNCTLNLT
29: ALNTTWTNLT
Library B:
  1: ANTTNFTNET
  2: ANWTNRTNCT
  3: ANWTNFTNWT
  4: PTGLIGTNFT
  5: ANWTNKTNFT
  6: ANNTNLTNAT
  7: ANYTNWTNFT
  8: ANTTNQTNDT
  9: -EXT wt
10: ANRTNWTNTT
11: PTATNHTNST
12: -EXT wt
13: ANWTNQTNQT
14: ANWTNWTNAT
15: ANFTNKTNMT
16: ANHTNETNAT
17: AN(C/W)TNFTNET
18: ANLDKLHKUH (insertion - nonsens)
19: ANCFTNQTNFT
20: ANWTNWTNEWT
21: ANCTNWTNCT
22: -EXT wt
23: -EXT wt
24: CHPYNWTNWT
25: ANETNYTNET
26: ANWTNWT
27: AKPYKSYKFY (insertion - nonsens)
28: ANITNKTNWT
29: ANWTNMTNIT
30: ANNTNRTNFT
31: ANWTNWTNWT
32: ANWRTNHTNKT
33: -EXT wt
34: ANQTNITNWT
```

Library B was transfected into insect cells using the Bac-N-Blue™ transfection kit from Invitrogen, Carlsbad, Calif., USA. First, 96 plaques from Library B were picked and tested by activity measurement (GCB Activity Assay). Plaques were selected as follows: 3 with high activity, 3 with medium activity and 3 with low or no activity, and virus was purified for DNA sequencing resulting in the following amino acid sequences:

High activity:
1-1: Mixed sequence
1-2: ANFTNVATNQT
1-3: (A)(N)TTXLTN(K)T
Medium activity:
2-1: ANKTN(S/C)TNIT
2-2: Mixed sequence
2-3: ANWTNCTN(I)T
Low activity:
3-1: ANWTN(F/L)TNWT
3-2: CQLDURSTNET
3-3: No sequence

From both libraries 96 plaques were picked and tested by activity measurement (GCB Activity Assay). From each library 6 plaques with high activity were selected and virus were purified for DNA sequencing. The amino acid sequence encoded by the different clones were:

Library A:
1: Mixed sequence
2: Mixed sequence
3: Mixed sequence
4: WT
5: ANNTNYTNWT
6: ANNTNYTNWT
Library B:
1: AANDTUNWTVNCT
2: ATNITLNYTANTT
3: WT
4: AANSTGNITINGT
5: AVNWTSNDTSNST

GCB polypeptides of the invention were tested for various properties, including GCB activity/stability in J774E cells and uptake in J774E cells. Unless otherwise stated the properties were tested by use of the methods described in the Methods section herein.

In the below table the GCB activity of various GCB polypeptides of the invention is listed together with the activity of the positives from Library A and B after plaque purification.

Table 2

TABLE 2
The plasmid column shows the number of the GCB polypeptide. The
vector column shows the plasmid vector used for expression of the polypeptide. The mutation
column shows the amino acid exchanges of the GCB polypeptide. N-terminal extentions are
described as N-term followed by the amino acid residues that makes up the extension. The
Activity column gives the units per liter of GCB activity measured by the GCB Activity Assay
on the supernatant from Sf9 insect cells infected with one single plaque and grown in 3 ml of
media in a 6-well plate. Those labelled with P2 are activity measured of supernatant from virus
infection cells grown in 15 ml T75 flasks.

| | | | | Activity after |
|---|---|---|---|---|
| | | | # Glycosylation | Plaque Isolation |
| Plasmid | Vector | Mutations | sites introduced | (U/L) |
| pGC-1 | PBlueBac4.5 | Wt | 0 | 6 |
| pGC-6 | pBlueBac4.5 | N-termANIT | 1 | 3 |
| pGC-12 | pVL1392 | Wt | 0 | 13 |
| pGC-13 | pVL1392 | N-termASPINAT | 1 | 29 |
| pGC-36 | pVL1392 | N-term: ASPINATSPINAT | 2 | 16 |
| pGC-38 | pVL1392 | N-term: ASPINAT, K194N, K321N | 3 | 16 |
| pGC-40 | pVL1392 | N-term: ASPINAT, T132N, K293N, V295T | 3 | 3.5 |
| pGC-47 | pVL1392 | N-term: AGNGTVNGTINGT | 3 | 30 |
| pGC-48 | pVL1392 | N-term: ASNSTNNGTLNAT | 3 | 36 |
| pGC-56 | pVL1392 | N-term: ASPINATSPINAT, K194N, K321N | 4 | 24 |
| pGC-57 | pVL1392 | N-term: ASPINAT, T132N, K194N, K321N | 4 | 20 |
| pGC-58 | pVL1392 | N-term: ASPINAT, T132N, K194N | 3 | 10 |
| pGC-60 | pVL1392 | N-term: ANNTNYTNWT | 3 | P2: 14 |
| pGC-61 | pVL1392 | N-term: ATNITLNYTANTT | 3 | P2: 38 |
| pGC-62 | pVL1392 | N-term: AANSTGNITINGT | 3 | P2: 35 |
| pGC-63 | pVL1392 | N-term: AVNWTSNDTSNST | 3 | P2: 66 |
| pGC-68 | pVL1392 | AN N-term extension —R2T | 1 | 37 |

Table 3

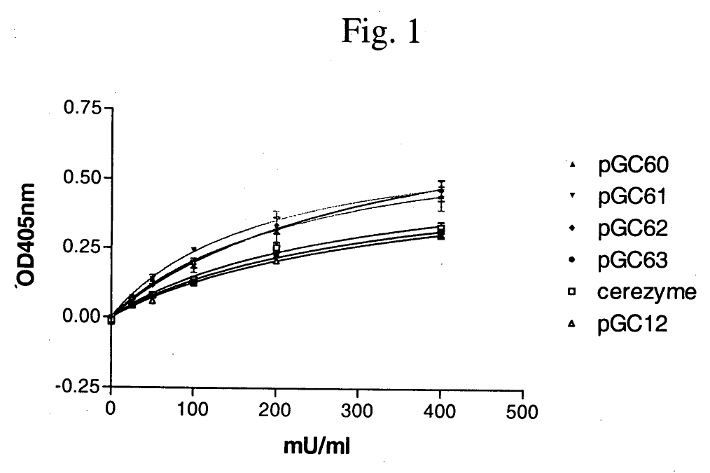

TABLE 3
Calculated Vmax and Km for uptake in the J774E macrophage cell line
of the different GCB polypeptides. Vmax and Km was calculated from
dosis response curve (See FIG. 1). The uptake of selected GCB
polypeptides are shown in FIG. 1. As can be seen from table 3, an
increase in Vₘₐₓ was observed for the N-terminally extended
GCB polypeptides (pGC60, pGC61, and pGC62).

| GCB polypeptide | Vmax | Km |
|---|---|---|
| Wildtype | 0.57 | 87.7 |
| Cerezyme | 0.52 | 91.9 |
| pGC36 | 0.60 | 70.6 |
| pGC38 | 0.48 | 44.0 |
| pGC56 | 0.39 | 32.2 |
| pGC60 | 0.57 | 79.1 |
| pGC61 | 0.74 | 100.5 |
| pGC62 | 0.86 | 110.8 |
| pGC63 | 0.51 | 83.1 |

### EXAMPLE 3

### Glycosylation of GCB Polypeptides of the Invention Expressed in Insect Cells

MALDI-TOF mass spectrometry was used to investigate the amount of carbohydrate attached to GCB polypeptides expressed in Sf9 cells.

The 6 GCB polypeptide variants investigated all contained additional potential N-glycosylation sites compared to wtGCB.

WtGCB contains 5 potential N-glycosylation sites of which only 4 are used.

The 6 GCB polypeptide variants were:

GC-36: ASPINATSPINAT-GCB.
GC-38: ASPINAT-GCB(K194N.K321N),
GC-60: ANNTNYTNWT-GCB,
GC-61: ATNITLNYTANTT-GCB,
GC-62: AANSTGNITINGT-GCB,
and
GC-63: AVNWTSNDTSNST-GCB.

WtGCB:

The theoretical peptide mass of wtGCB is 55 591 Da. WtGCB has 5 potential N-glycosylation sites of which only 4 are used. As the two most common N-glycan structures on recombinant proteins expressed in Sf9 cells are Man₃GlcNAc₂Fuc and Man₃GlcNAc₂ having masses of 1038.38 Da and 892.31 Da, respectively, the expected mass of wtGCB carrying 4 N-glycans is between 59 159 Da and 59 743 Da.

MALDI-TOF mass spectrometry of wtGCB shows the broad peak typical of glycoproteins with a peak mass of 59.3 kDa in accordance with the expected mass of wtGCB carrying 4 N-glycans.

GC-36 (ASPINATSPINAT-GCB):

The theoretical peptide mass of GC-36 is 56 829 Da. The N-terminal extension contains two additional potential glycosylation sites at N5 and N11 compared to wtGCB. Assuming that the wtGCB part of the variant is glycosylated like wtGCB, the variant has 6 potential N-glycosylation sites.

As the two most common N-glycan structures on recombinant proteins expressed in Sf9 cells are Man₃GlcNAc₂Fuc and Man₃GlcNAc₂ having masses of 1038.38 Da and 892.31 Da, respectively, the expected mass of GC-36 carrying 4 N-glycans is between 60 397 Da and 60 981 Da, the expected mass of GC-36 carrying 5 N-glycans is between 61 289 Da and 62 019 Da, and the expected mass of GC-36 carrying 6 N-glycans is between 62 181 Da and 63 057 Da.

MALDI-TOF mass spectrometry of GC-36 shows a rather broad peak with a peak mass between 61.5 kDa and 62.9 kDa in accordance with the expected mass of GC-36 carrying either 5 or 6 N-glycans.

N-terminal amino acid sequence analysis of GC-36 showed that N5 is completely glycosylated while N11 is partially glycosylated in complete agreement with the result obtained using mass spectrometry.

GC-38 (ASPINAT-GCB(K194N,K321N)):

The theoretical peptide mass of GC-38 is 56 217 Da. The N-terminal extension contains one additional potential glycosylation sites at N5 compared to wtGCB. In addition, the substitutions of Lys194 and Lys321 with Asn-residues introduce two additional potential N-glycosylation sites. Assuming that the wtGCB part of the variant is glycosylated like wtGCB, the variant has 7 potential N-glycosylation sites.

Based on the same considerations as those used for GC-36, the expected mass of GC-38 carrying 4 N-glycans is between 59 785 Da and 60 369 Da, the expected mass of GC-38 carrying 5 N-glycans is between 60 677 Da and 61 407 Da, the expected mass of GC-38 carrying 6 N-glycans is between 61 569 Da and 62 445 Da, and the expected mass of GC-38 carrying 7 N-glycans is between 62 461 Da and 63 483 Da.

MALDI-TOF mass spectrometry of GC-38 shows a major peak with a peak mass of 63.1 kDa in accordance with the expected mass of GC-38 carrying 7 N-glycans. In addition, a minor peak with a peak mass of 62.3 kDa is seen which corresponds to GC-38 carrying 6 N-glycans.

N-terminal amino acid sequence analysis of GC-38 showed that N5 is completely glycosylated.

GC-60 (ANNTNYTNWT-GCB):

The theoretical peptide mass of GC-60 is 56 770 Da. The N-terminal extension contains three additional potential glycosylation sites at N2, N5 and N8 compared to wtGCB. Assuming that the wtGCB part of the variant is glycosylated like wtGCB, the variant has 7 potential N-glycosylation sites.

Based on the same considerations as those used for GC-36 the expected mass of GC-60 carrying 4 N-glycans is between 60 338 Da and 60 922 Da, the expected mass of GC-60 carrying 5 N-glycans is between 61 230 Da and 61 960 Da, the expected mass of GC-60 carrying 6 N-glycans is between 62 122 Da and 62 998 Da, and the expected mass of GC-60 carrying 7 N-glycans is between 63 014 Da and 64 036 Da.

MALDI-TOF mass spectrometry of GC-60 shows two broad peaks with peak masses of 61.9 kDa and 62.8 kDa in accordance with the expected mass of GC-60 carrying either 5 or 6 N-glycans.

N-terminal amino acid sequence analysis of GC-60 showed that N2 is mainly glycosylated, N5 is completely glycosylated while N8 is only seldom glycosylated in acceptable agreement with the result obtained using mass spectrometry.

GC-61 (ATNITLNYTANTT-GCB):

The theoretical peptide mass of GC-61 is 56 970 Da. The N-terminal extension contains three additional potential glycosylation sites at N3, N7 and N11 compared to wtGCB. Assuming that the wtGCB part of the variant is glycosylated like wtGCB, the variant has 7 potential N-glycosylation sites.

Based on the same considerations as used for GC-36, the expected mass of GC-61 carrying 4 N-glycans is between 60 538 Da and 61 122 Da, the expected mass of GC-61 carrying 5 N-glycans is between 61 430 Da and 62 160 Da, the expected mass of GC-61 carrying 6 N-glycans is between 62 322 Da and 63 198 Da, and the expected mass of GC-61 carrying 7 N-glycans is between 63 214 Da and 64 236 Da.

MALDI-TOF mass spectrometry of GC-61 shows a very broad peak with peak mass between 61.5 kDa and 63.0 kDa in accordance with the expected mass of GC-61 carrying either 5 or 6 N-glycans.

N-terminal amino acid sequence analysis of GC-61 showed that N3 is completely glycosylated while N7 and N11 are partially glycosylated in acceptable agreement with the result obtained using mass spectrometry.

GC-62 (AANSTGNITINGT-GCB):

The theoretical peptide mass of GC-62 is 56 806 Da. The N-terminal extension contains three additional potential glycosylation sites at N3, N7 and N I compared to wtGCB. Assuming that the wtGCB part of the variant is glycosylated like wtGCB, the variant has 7 potential N-glycosylation sites.

Based on the same considerations as those used for GC-36, the expected mass of GC-62 carrying 4 N-glycans is between 60 374 Da and 60 958 Da, the expected mass of GC-62 carrying 5 N-glycans is between 61 266 Da and 61 996 Da, the expected mass of GC-62 carrying 6 N-glycans is between 62 158 Da and 63 034 Da, and the expected mass of GC-62 carrying 7 N-glycans is between 63 05.0 Da and 64 072 Da.

MALDI-TOF mass spectrometry of GC-62 shows two broad peaks with peak masses of 61.6 kDa and 62.7 kDa in accordance with the expected mass of GC-62 carrying either 5 or 6 N-glycans.

N-terminal amino acid sequence analysis of GC-62 showed that N3 is completely glycosylated while N7 and N11 are partially glycosylated in acceptable agreement with the result obtained using mass spectrometry.

GC-63 (AVNWTSNDTSNST-GCB):

The theoretical peptide mass of GC-63 is 56 969 Da. The N-terminal extension contains three additional potential glycosylation sites at N3, N7 and N1 compared to wtGCB. Assuming that the wtGCB part of the variant is glycosylated like wtGCB, the variant has 7 potential N-glycosylation sites.

Based on the same considerations as those used for GC-36, the expected mass of GC-63 carrying 4 N-glycans is between 60 537 Da and 61 121 Da, the expected mass of GC-63 carrying 5 N-glycans is between 61 429 Da and 62 159 Da, the expected mass of GC-63 carrying 6 N-glycans is between 62 321 Da and 63 197 Da, and the expected mass of GC-63 carrying 7 N-glycans is between 63 213 Da and 64 235 Da.

MALDI-TOF mass spectrometry of GC-63 shows a major peak with a peak mass of 61.9 kDa in accordance with the expected mass of GC-63 carrying 5 N-glycans. In addition, a minor peak with a peak mass of 62.9 kDa is seen which corresponds to GC-63 carrying 6 N-glycans.

N-terminal amino acid sequence analysis of GC-63 showed that N3 ans N7 are partially glycosylated. It was not possible to evaluate the glycosylation status of N11.

Furthermore, insect cell expressed N-terminally extended glycosylated polypeptide (GC-6 and GC-13) was subjected to N-terminal amino acid sequence analysis (using Procize from PE Biosystems, Foster City, Calif.). The sequencing cycle was blank for the Asn residue in both ANIT and ASPINAT N-terminal peptide additions, demonstrating that the introduced glycosylation site is glycosylated.

When subjecting GC-13 to mass spectrophometry using the MALDI-TOF techniques on the Voyager DERP instrument (from PE-Biosystems, Foster City, Calif.) the following results were obtained:

The wildtype and ASPINAT-extended wildtype expressed in insect cells gave average masses very close to the calculated mass of 59,727 Da and 61,421 Da, respectively, assuming that four glycosylation sites were occupied by the carbohydrates FucGlcNAc₂Man₃.

### EXAMPLE 4

### Construction of Plasmids for Expression of FSH

A gene encoding the human FSH-alpha subunit was constructed by assembly of synthetic oligonucleotides by PCR using methods similar to the ones described in Stemmer et al. (1995) Gene 164, pp. 49-53. The native FSH-alpha signal sequence was maintained in order to allow secretion of the gene product. The codon usage of the gene was optimised for high expression in mammalian cells. Furthermore, in order to achieve high gene expression, an intron (from pCI-Neo (Promega)) was included in the 5′ untranslated region of the gene. The synthetic gene was subcloned behind the CMV promoter in pcDNA3.1/Hygro (Invitrogen). The sequence of the resulting plasmid, termed pBvdH977, is given in SEQ ID NO:3 (FSH-alpha-coding sequence at position 1225 to 1570). Similarly, a synthetic gene encoding the wildtype-human FSH-beta subunit was constructed. Also in this construct, the native signal sequence was maintained (except for a Lys to Glu mutation at position 2) in order to allow secretion, and the codon usage was optimised for high expression and an intron was included in the recipient vector (pcDNA3.1/Zeo (Invitrogen)). The sequence of the resulting FSH-beta-containing plasmid, termed pBvdH1022, is given in SEQ ID NO:4 (FSH-beta-coding sequence at position 1231 to 1617). A plasmid containing both the FSH-alpha and the FSH-beta encoding synthetic genes was generated by subcloning the FSH-alpha containing NruI-PvuII fragment from pBvdH977 into pBvdH1022 linearized with NruI. The resulting plasmid, in which the FSH-alpha and FSH-beta-expression cassettes are in direct orientation, was termed pBvdH 1100.

Expression of FSH in CHO Cells

FSH was expressed in Chinese Hamster Ovary (CHO) K1 cells, obtained from the American Type Culture Collection (ATCC, CCL-61).

For transient expression of FSH, cells were grown to 95% confluency in serum-containing media (MEMA with ribonucleotides and deoxyribonucleotides (Life Technologies Cat #32571-028) containing 1:10 FBS (BioWhittaker Cat # O₂-701F) and 1:100 penicillin and streptomycin (BioWhittaker Cat #17-602E), or Dulbecco's MEM/Nut.-mix F-12 (Ham) L-glutamine, 15 mM Hepes, pyridoxine-HCl (Life Technologies Cat # 31330-038) with the same additives. FSH-encoding plasmids were transfected into the cells using Lipofectamine 2000 (Life Technologies) according to the manufacturer's specifications. 24-48 hrs after transfection, culture media were collected, centrifuged and filtered through 0.22 micrometer filters to remove cells.

Stable clones expressing FSH were generated by transfection of CHO K1 cells with FSH-encoding plasmids followed by incubation of the cells in selective media (for instance one of the above media containing 0.5 mg/ml zeocin for cells transfected with plasmid pBvdH 1100). Stably transfected cells were isolated and sub-cloned by limited dilution. Clones that produced high levels of FSH were identified by ELISA.

More specifically, the concentration of FSH in samples was quantified by use of a commercial immunoassay (DRG FSH EIA, DRG Instruments GmbH, Marburg, Germany). DRG FSH EIA is a solid phase immunosorbent assay (ELISA) based on the sandwich principle. The microtiter wells are coated with a monoclonal antibody directed towards a unique antigenic site on the FSH-β subunit. An aliquot of FSH-containing sample (diluted in H₂O with 0.1% BSA) and an anti-FSH antiserum conjugated with horseradish peroxidase are added to the coated wells. After incubation, unbound conjugate is washed off with water. The amount of bound peroxidase is proportional to the concentration of FSH in the sample. The intensity of colour developed upon addition of substrate solution is proportional to the concentration of FSH in the sample.

Large-Scale Production of FSH in CHO Cells

The cell line CHO K1 1100-5, stably expressing human FSH, was passed 1:10 from a confluent culture and propagated as adherent cells in serum-containing medium Dulbecco's MEM/Nut.-mix F-12 (Ham) L-glutamine, 15 mM Hepes, pyridoxine-HCl (Life Technologies Cat # 31330-038), 1:10 FBS (BioWhittaker Cat # O₂-701F), 1:100 penicillin and streptomycin (BioWhittaker Cat # 17-602E) until confluence in a 10 layer cell factory (NUNC #165250). The media was then changed to serum-free media: Dulbecco's MEM/Nut.-mix F-12 (Ham) L-glutamine, pyridoxine-HCl (Life Technologies Cat # 21041-025) with the addition of 1:500 ITS-A (Gibco/BRL # 51300-044), 1:500 EX-CYTE VLE (Serological Proteins Inc. # 81-129) and 1:100 penicillin and streptomycin (BioWhittaker Cat # 17-602E). Subsequently, every 24 h, culture media were collected and replaced with 1 fresh liter of the same serum-free media. The collected media was filtered through 0.22 μm filters to remove cells. Growth in cell factories was continued with daily harvests and replacements of the culture media until FSH yields dropped below one-fourth of the initial expression level (typically after 10-15 days).

### EXAMPLE 5

### Purification of FSH Wildtype and Variants

Three chromatographic steps were employed to obtain highly purified FSH. First an anion exchanger step, then hydrophobic interaction chromatography (HIC) and finally an immunoaffinity step using an FSH-β specific monoclonal antibody.

Culture supernatants were prepared as described in Example 4. Filtered culture supernatants were concentrated 10 to 20 times by ultrafiltration (10 kD cut-off membrane), pH was adjusted to 8.0 and conductivity to 10-15 mS/cm, before application on a DEAE Sepharose (Pharmacia) anion exchanger column, which had been equilibrated in ammonium acetate buffer (0.16 M, pH 8.0). Semipurified FSH was recovered both in the unbound flow-through fraction as well as in the wash fraction using 0.16 M ammonium acetate, pH 8.0. The flow through and wash fractions were pooled and ammonium sulfate was added from a stock solution (4.5 M) to obtain a final concentration of 1.5 M (NH₄)₂SO₄. The pH was adjusted to 7.0.

The partially purified FSH was subsequently applied on a 25 ml butyl Sepharose (Pharmacia) HIC column. After application, the column was washed with at least 3 column volumes of 1.5 M (NH₄)₂SO₄, 20 mM ammonium acetate, pH 7 (until the absorbance at 280 nm reached baseline level) and FSH was eluted with 4 column volumes of buffer B (20 mM ammonium acetate, pH 7). FSH enriched fractions from the HIC step were pooled, concentrated and diafiltrated using Vivaspin 20 modules, 10 kD cut-off membrane (Vivascience), to a 50 mM sodium phosphate, 150 mM NaCl, pH 7.2.

For the third chromatographic step, an anti-FSH-β monoclonal antibody (RDI-FSH909, Research Diagnostics) was immobilized to CNBr-activated Sepharose (Pharmacia) using a standard procedure from the supplier. Approximately 1 mg antibody was coupled per ml resin. The immunoaffinity resin was packed in plastic columns and equilibrated with 50 mM sodium phosphate, 150 mM NaCl, pH 7.2 before application.

The buffer exchanged eluate from the butyl HIC step was applied on the antibody column by use of gravity flow. This was followed by several washing steps in 50 mM sodium phosphate solutions (0.5 M NaCl and 1 M NaCl, both pH 7.2). Elution was performed using either 1 M NH₃ or 0.6 M NH₃, 40% (v/v) isopropanol and the eluate was immediately neutralized with 1 M acetic acid to pH 6-8.

The purified FSH bulk product was concentrated and diafiltrated using Vivaspin 20 modules, 10 kD cut-off membrane (Vivascience), to a 50 mM sodium phosphate, 150 mM NaCl, pH 7.2. For subsequent storage, BSA was added to 0.1% (w/v) and the purified FSH was microfiltrated using a 0.22 μm filter prior to storage at −80° C.

SDS-PAGE, run under non-dissociating conditions (without boiling), showed wildtype FSH migrating as an apparant 42±3 kDa band, slightly diffuse due to heterogeneity in the attached carbohydrates. The purity was about 80-90%. N-terminal sequencing showed that the α-chain had the expected N-terminal sequence starting with residue 1 (SEQ ID NO:5) and the β-chain starting with residue 3 (SEQ ID NO:6). These N-terminal sequences have been found previously for recombinant FSH produced in CHO cells (Olijve, W. et al. (1996) Mol. Hum. Reprod. 2, 371-382).

### EXAMPLE 6

### FSH in Vitro Activity Assay

6.1 FSH Assay Outline

It has previously been published that activation of the FSH receptor by FSH leads to an increase in the intracellular concentration of cAMP. Consequently, transcription is activated at promoters containing multiple copies of the cAMP response element (CRE). It is thus possible to measure FSH activity by use of a CRE luciferase reporter gene introduced into CHO cells expressing the FSH receptor.

6.2 Construction of a CHO FSH-R/CRE-luc Cell Line

Stable clones expressing the human FSH receptor were produced by transfection of CHO K1 cells with a plasmid containing the receptor cDNA inserted into pcDNA3 (Invitrogen) followed by selection in media containing 600 microg/ml G418. Using a commercial cAMP-SPA RIA (Amersham), clones were screened for the ability to respond to FSH stimulation. On the basis of these results, an FSH receptor-expressing CHO clone was selected for further transfection with a CRE-luc reporter gene. A plasmid containing the reporter gene with 6 CRE elements in front of the Firefly luciferase gene was co-transfected with a plasmid conferring Hygromycin B resistance. Stable clones were selected in the presence of 600 microg/ml G418 and 400 microg/ml Hygromycin B. A clone yielding a robust luciferase signal upon stimulation with FSH (EC₅₀˜0.01 IU/ml) was obtained. This CHO FSH-R/CRE-luc cell line was used to measure the activity of samples containing FSH.

6.3 FSH Luciferase Assay

To perform activity assays, CHO FSH-R/CRE-luc cells were seeded in white 96 well culture plates at a density of about 15,000 cells/well. The cells were in 100 l DMEM/F-12 (without phenol red) with 1.25% FBS. After incubation overnight (at 37° C., 5% CO₂), 25 μl of sample or standard diluted in DMEM/F-12 (without phenol red) with 10% FBS was added to each well. The plates were further incubated for 3 hrs, followed by addition of 125 μl LucLite substrate (Packard Bioscience). Subsequently, plates were sealed and luminescence was measured on a TopCount luminometer (Packard) in SPC (single photon counting) mode.

### EXAMPLE 7

### Construction and Analysis of a Variant Form of FSH Containing Two N-Linked Glycosylations at the N-Terminus of the Alpha Subunit

A construct encoding a modified form of FSH-alpha, having two additional sites for N-linked glycosylation at its N-terminus was generated by site-directed mutagenesis using standard DNA techniques known in the art. A DNA fragment encoding the sequence Ala-Asn-Ile-Thr-Val-Asn-Ile-Thr-Val was inserted immediately upstream of the mature FSH-alpha sequence in pBvdH977. The sequence of the resulting plasmid, termed pBvdH 1163, is given in SEQ ID NO:7 (modified FSH-alpha-encoding sequence at position 1225 to 1599). A plasmid encoding both subunits was constructed by subcloning the FSH-containing NruI-PvuII fragment from pBvdH 1163 into pBvdH1022 (Example 4), which had been linearized with PvuII. The resulting plasmid was termed pBvdH1208.

For expression of the variant form of FSH containing two N-linked glycosylations at the N-terminus of the alpha subunit (termed FSH1208), CHO K1 cells were transfected with pBvdH1208 or co-transfected with a combination of pBvdH 163, encoding the modified alpha subunit and pBvdH1022, encoding the wildtype beta subunit. Transient expressions, isolation of stable expression clones, and large-scale production of FSH1208 were performed as described for wildtype FSH in Example 4.

The FSH content of samples was analysed by Western Blotting: Proteins were separated by SDS-PAGE and a standard Western blot was performed using rabbit anti human FSH (AHP519, Serotec) or mouse anti human FSH-alpha (MCA338, Serotec) as primary antibody, and an ImmunoPure Ultra Sensitive ABC Peroxidase Staining Kit (Pierce) for detection. Western blotting showed that FSH1208 had a larger molecular mass than wildtype FSH, indicating that the introduction of acceptor sites for N-linked glycosylation at the N-terminus of the alpha subunit indeed lead to hyperglycosylation of FSH. For analysis of pI, samples were separated on pH 3-7 IEF gels (NOVEX). After electrophoresis, proteins were blotted onto Immobilon-P (Millipore) membranes and a Western blot was performed as described above, using the same antibodies and detection kit. Isoelectric focusing demonstrated that the FSH forms in the FSH1208 samples were found in a lower pI range than wildtype FSH. Thus, the pH interval for FSH1208 isoforms was about 3.0-4.5 versus about 4.0-5.2 for wildtype FSH. This indicated that FSH1208 molecules are on average more negatively charged than the wild type, which is attributed to the presence of additional sialic acid residues.

FSH1208 was purified and characterized as described in Example 5. SDS-PAGE, run under non-dissociating conditions (without boiling), showed FSH1208 migrating as an apparent 55±5 kDa band, slightly diffuse due to heterogeneity in the attached carbohydrates. The purity was about 80-90%. N-terminal sequencing showed that while the β-chain had the same N-terminal sequence as wildtype FSH, the sequence of α-chain was in agreement with this subunit carrying the expected N-terminal extension ANITVNITV, in which both asparagines residues are glycosylated.

The specific activity of FSH1208 was determined by measurement of the in vitro bioactivity (FSH luciferase assay, Example 6) and the FSH content of the samples by ELISA. The specific activity of FSH1208 was found to be about one-third of that of the wildtype reference.

A pharmacokinetic study performed as follows:

Immature 26-27 days old female Sprague-Dawley rats were injected i.v. with 3-4 microg FSH, produced, purified and analyzed as described above. Subsequently, blood samples were taken at various time-points after injection. FSH concentrations in serum samples were determined by ELISA, as described above.

In vivo bioactivity of wildtype recombinant FSH and variant forms may be evaluated by the ovarian weight augmentation assay (Steelman and Pohley (1953) Endocrinology 53, 604-616). Furthermore, the ability of FSH and variant forms to stimulate maturation of follicles in laboratory animals may be detected with e.g., ultrasound equipment. The experiment showed that 24 hours after injection of equal amounts of wildtype FSH and FSH1208, the sera of FSH 1208-treated animals contained more than 10 fold more remaining immunoreactive material than the sera from animals treated with wildtype FSH.

### EXAMPLE 8

### Construction and Analysis of Other FSH Variants Containing Additional Glycosylation Sites

Plasmids encoding variant forms of FSH-alpha and FSH-beta containing additional sites for N-linked glycosylation were generated by site-directed mutagenesis using standard DNA techniques known in the art. The following amino acid substitutions and/or insertions were generated:

FSH1147: Amino acid Tyr58 of mature FSH-beta altered to Asn

FSH1349: N-terminus of mature FSH-alpha altered from APD QDC . . . to: APNDTVNFT QDC . . .

FSH1354: N-terminus of mature FSH-beta altered from NS CEL . . . to: NSNITVNITV CEL . . .

Plasmids encoding the variant forms were transiently expressed in CHO K1 cells as described in Example 4. Plasmids encoding FSH-alpha variants were co-transfected with a plasmid encoding wild-type FSH-beta and vice versa.

Western and isoelectric focusing were performed on culture media samples as described above. The variant forms had higher molecular weights than the wild-type, indicating that the additional acceptor sites for N-linked glycosylation had indeed been glycosylated. Furthermore, isoelectric focusing showed that the different isoforms of the three FSH variants were spread over a lower pI range than the wildtype. This strongly suggests that the variant forms had a higher sialic acid content than the wildtype.

In vitro FSH activities of the resulting media samples were analysed as described in Example 6.3. All three variant forms were able to stimulate the CHO FSH-R/CRE-luc cells, indicating that these variant FSH forms have retained significant FSH activity.

While the foregoing invention has been described in some detail for purposes of clarity and understanding, it will be clear to one skilled in the art from a reading of this disclosure that various changes in form and detail can be made without departing from the true scope of the invention. For example, all the techniques, methods, compositions, apparatus and systems described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

1.-57. (canceled)

58. A method for increasing the serum half-life of a mature form of a polypeptide of interest, the method comprising:
a) preparing a nucleic acid comprising a nucleotide sequence encoding a peptide-extended polypeptide with the primary structure NH₂-X-Pp-COOH, wherein
NH₂ and COOH represent the N-terminus and the C-terminus of the peptide-extended polypeptide, respectively;
X is a peptide addition 1-30 consecutive amino acids in a linear chain, wherein X comprises or contributes to an in vivo N-glycosylation site; and
Pp is the sequence of the mature form of the polypeptide of interest, wherein X and Pp are linked by a peptide linkage; and
b) expressing the nucleic acid in a mammalian host cell to provide a peptide-extended glycosylated polypeptide;
wherein the peptide-extended glycosylated polypeptide exhibits an altered glycosylation pattern and an increased serum half-life compared to that of the mature form of the polypeptide of interest when expressed under the same conditions.

59. The method of claim 58, further comprising:
recovering the peptide-extended glycosylated polypeptide.

60. The method of claim 58, wherein the polypeptide of interest is a cytokine or a hormone.

61. The method of claim 58, wherein X is of the formula:
X₁′-N-X₂-[T/S]/C-Z wherein
X₁′ is absent or comprises at least one amino acid;
X₂ is any one amino acid except proline;
Z is absent or comprises at least one amino acid;
N is asparagine; and
[T/S]/C is threonine, serine, or cysteine.

62. The method of claim 61, wherein X is of the formula:
X₁-(N-X₂-[T/S])-X₃-(N-X₂-[T/S])ₙ-X₄ wherein
X₁ is absent, or is any 1, 2, 3, or 4 amino acids;
X₂ is any one amino acid except proline;
X₃ is absent, or is any 1, 2, 3, or 4 amino acids;
X₄ is absent, or is any 1, 2, 3, or 4 amino acids;
n is an integer between 0 and 6; N is asparagine; and [T/S] is threonine or serine.

63. The method of claim 61, wherein
X₂ is isoleucine, alanine, glycine, valine, or serine.

64. The method of claim 58, wherein the mammalian host cell is selected from a hamster cell line, a monkey cell line, a mouse cell line, a hamster cell line, and a human cell line.

65. The method of claim 64, wherein the mammalian host cell is a CHO cell.

66. The method of claim 64, wherein the mammalian host cell is selected from CHO-K1, COS 1, COS 7, NS/O, BHK, and HEK 293.

67. The method of claim 59, further comprising:
incubating the peptide-extended glycosylated polypeptide with a non-peptide moiety which differs from an oligosaccharide moiety, under conditions suitable to covalently attach said non-peptide moiety to an attachment group of the polypeptide.
